(19) United States
(12) Patent Application Publication
(10) Pub. No.: US 2015/0342893 A1
(43) Pub. Date: Dec. 3, 2015

(54) CELECOXIB FORMULATIONS USEFUL FOR TREATING COLORECTAL CANCER

(71) Applicant: SIGMOID PHARMA LIMITED, Dublin (IE)

(72) Inventors: Ivan Coulter, Dublin (IE); Bernard Francis McDonald, Castleblayney (IE); Vincenzo Aversa, Swords (IE)

(73) Assignee: SIGMOID PHARMA LIMITED, Dublin (IE)

(21) Appl. No.: 14/723,314

(22) Filed: May 27, 2015

Related U.S. Application Data
(60) Provisional application No. 62/004,784, filed on May 29, 2014.

(51) Int. Cl. A61K 9/48; A61K 45/06; A61K 31/635

(52) U.S. Cl. CPC A61K 9/4891; A61K 31/635; A61K 9/4866; A61K 9/4858; A61K 45/06; A61K 9/4833; A61K 9/485

(57) ABSTRACT

An oral celecoxib formulation, the formulation being a multiple minibead formulation wherein the minibeads comprise a hydrogel-forming polymer matrix in which are distributed celecoxib, a polyoxyethylated non-ionic surfactant and an anionic surfactant, the minibeads when combined with water being capable of releasing self-assembly structures comprising surfactant and celecoxib.

The formulation may be used for treating colorectal cancer, e.g. for inhibiting, reducing or delaying the initiation and/or progression of colorectal cancer, or for use in reversing colorectal cancer, reducing the burden of colorectal cancer and/or inducing remission of colorectal cancer, or for inhibiting, reducing or delaying metastasis of a colorectal cancer.

### CROSS-REFERENCE TO RELATED APPLICATIONS

This application claims the benefit of U.S. Provisional Application No. 62/004,784, filed on May 29, 2014. The provisional application is incorporated herein by reference in its entirety.

This invention relates to celecoxib formulations that are useful for the therapy and prophylaxis of colorectal cancer and other diseases. The invention further relates to the manufacture and use of such compositions, and to other subject matter.

### BACKGROUND

Celecoxib is a poorly soluble drug. It is a cyclooxygenase-2 inhibitor (COX-2) inhibitor, weakly acidic (pKa 11.1), hydrophobic in nature (Log P 3.5) and has a low aqueous solubility of 3-7 μg/ml at 20° C. Celecoxib is routinely administered in the treatment of osteoarthritis, adult rheumatoid arthritis and ankylosing spondylitis (Homar et al., Journal of Microencapsulation, 2007; 24(7): 621-633). Celecoxib has also demonstrated significant chemopreventative activity in colon carcinogenesis (Maier et al., Biochem. Pharmacol., 2004; 67: 1469-1478; Reddy et al., Cancer Res., 2000; 60: 293-297; Kawamori et al., Cancer Res., 1998; 58: 409-412). The administration of celecoxib is associated with the potential risk for serious cardiovascular (CV) and/or gastrointestinal (GI) adverse events (Sostres et al., Best Practice & Research Clinical Gastroenterology, 2010; 24: 121-132). Bioavailability studies have shown celecoxib to be a poorly soluble, highly permeable drug, (i.e., class II of the Biopharmaceutical Classification System), in which the bioavailability of the drug is limited by its poor solubility (Paulson et al., Journal of Pharmacology and Experimental Therapeutics, 2001; 297 (2): 638-645). This poor aqueous solubility, and consequent poor dissolution in gastric fluids is the major drawback of celecoxib therapy (Rawat, European Journal of Pharmaceutics and Biopharmaceutics, 2004; 57: 263-267).

Colorectal cancer (CRC) is the third most common cause of cancer mortality worldwide with more than 1 million new cases of CRC diagnosed each year. CRC is a heterogeneous disease, including at least three major forms; hereditary, sporadic, and colitis-associated CRC. Together with familial adenomatous polyposis (FAP) and hereditary nonpolyposis colorectal cancer (HNPCC), chronic inflammation is among the top three high risk conditions for CRC. An abnormal gastrointestinal immune response is believed to result in chronic inflammation such as inflammatory bowel disease (IBD). IBD is a complex class of immune disorders that have been grouped into two major forms, ulcerative colitis (UC) and Crohn's Disease (CD). Colitis-associated cancer (CAC) is the CRC subtype that is associated with IBD, it is difficult to treat and has a high mortality. More than 20% of IBD patients develop CAC within 30 years of disease onset and >50% will die from CAC.

Significant research has been dedicated to identify novel drug targets for CRC prevention and treatment. Non-steroidal anti-inflammatory drugs (NSAIDs) are one group of compounds that have been found to decrease the risk of CRC. NSAIDs target and inhibit the cyclooxgenase enzymes, COX-1 and COX-2. Since elevated COX-2 expression has been found in approximately 50% of colorectal adenomas and 85% of colorectal adenocarcinomas, it is hypothesised that NSAIDs may exert some of their anti-inflammatory and anti-tumour effects through inhibition of COX-2. Following this hypothesis, and the fact that many of the unwanted gastrointestinal side effects associated with NSAIDs are related to COX-1 inhibition, there has been a focus on the use of COX-2 selective NSAIDs for the treatment and prevention of CRC. As indicated above, celecoxib is such a drug. The poor solubility of the drug, however, reduces its effectiveness in treating the diseases for which it is indicated. In order to counteract this poor bioavailability, the drug is administered in high doses which may thereby result in more CV and GI adverse side effects. In the case of GI side effects, the administration of the drug in a soluble form and at a lower dose has the potential to alleviate GI irritation (Sacchetti, A. J Cell Biochem. 2013; 114(6):1434-44), whereas CV side effects could be reduced by lowering the systemic dose (Solomon SD et al. N Engl J Med 2005; 352:1071-1080).

WO2010/133609 of Sigmoid Pharma Limited discloses a composition that can be described as a dried oil-in-water emulsion that may be in the shape of minibeads, wherein the water phase of the emulsion comprises gelatin or another water-soluble polymer that forms a matrix in the dried composition. It is taught that lipophilic active principles may be solubilised in the oil phase. WO2010/133609 further teaches as follows in [095]: “The oil phase preferably also comprises a co-solvent for the active principle (particularly in the case of poorly-soluble active principles such as for example cyclosporine or celecoxib).” A recited example of a suitable co-solvent is 2-(2-ethoxyethoxy)ethanol.

### BRIEF SUMMARY OF THE DISCLOSURE

The full ambit of the invention is disclosed in the following specification and paragraphs, when read in conjunction with the above background. To assist the reader, however, a brief and non-limiting overview of the invention is contained in this paragraph and the paragraph immediately following. These paragraphs are to be read together with the remainder of the disclosure and with the above background. The invention provides (amongst other things) a liquid comprising water, solubilised celecoxib, a polyethoxylated non-ionic surfactant and a hydrogel-forming polymer, and the dried liquid in the form of minibeads. Without being bound by theory, the minibeads are believed to comprise a dried colloid, particularly a dried micellar solution, wherein the colloid has a continuous aqueous phase and a discontinuous surfactant phase, wherein the aqueous phase comprises gelatin or another hydrogel-forming polymer, and the discontinuous phase comprises the polyethoxylated non-ionic surfactant and celecoxib. The dried liquid (dried colloid) also comprises a precipitation inhibitor. The polymer forms a polymer matrix in the dried liquid. The precipitation inhibitor may be an anionic surfactant, for example an alkyl sulfate salt, especially SDS. The non-ionic surfactant may be a combination of mono- and di-poly(oxyethylene) esters of a hydroxy fatty acid (together with free PEG). The non-ionic surfactant may be macrogol-15-hydroxystearate, which is also known as polyoxyl-15-hydroxystearate. The dried colloid may contain little or no 2-(2-ethoxyethoxy)ethanol. The dried liquid may contain little or no oil. The dried liquid may contain little or no lipid and little or no hydrocarbon. The dried liquid may contain little or no 2-(2-ethoxyethoxy)ethanol, little or no lipid, and little or no hydrocarbon. The minibeads are obtainable by converting into minibeads a clear aqueous liquid comprising gelatin or another hydrogel-forming polymer, the anionic surfactant, the precipitation inhibitor and celecoxib. The clear aqueous liquid may be made by combining, for example, an aqueous premix comprising, or consisting essentially of, water, the polymer, the anionic surfactant and an optional plasticiser with a non-aqueous premix comprising, or consisting essentially of, the non-ionic surfactant and celecoxib. The products and methods disclosed in this paragraph are part of the invention and therefore may be claimed, even though the invention is not at all limited to the subject matter of this paragraph.

The Examples of this specification and the accompanying drawings contain data indicating that the minicapsules of the invention have the following effects: (i) they inhibit proliferation of colorectal cancer cells to a much greater extent than current marketed product Celebrex®; (ii) they selectively kill colorectal cancer cells by apoptosis in a test where Celebrex® had no significant impact on cell viability; and (iii) they reduce the potential of colorectal cancer cells to metastasise. A multiple minibead formulation of the invention that targets release of celecoxib to the colon may therefore be used for the therapeutic or prophylactic treatment of colorectal cancer, particularly a COX-2 positive adenocarcinoma, at a lower dose and reduced side-effects compared with what would be possible with Celebrex®. By virtue of the effect of multiple minibead formulations of the invention in releasing celecoxib in active form such that a low drug dose may advantageously be chosen, a celecoxib formulation is provided that, for other indications and disorders (e.g. anti-inflammatory treatment) may be therapeutically effective with a low level of side effects, providing therefore a beneficial advance on what is currently available in the clinic.

In one aspect, the invention provides a liquid composition useful for making the pharmaceutical formulations of the invention. The invention therefore provides in particular a composition that is a clear liquid comprising water, a hydrogel-forming polymer, celecoxib, a polyoxyethylated non-ionic surfactant and a precipitation inhibitor, wherein the celecoxib is in an amount of at least 2% by weight of the dry weight of the composition.

The invention includes within its scope a composition that is a clear liquid comprising water, a hydrogel-forming polymer, celecoxib, a polyoxyethylated non-ionic surfactant and a precipitation inhibitor, wherein the celecoxib is in an amount of at least 2% and the non-ionic surfactant is in an amount of at least 40%, for example at least 50%. The composition may contain less than 10% triglyceride. All percentages in this paragraph are by weight based on the dry weight of the composition.

The invention includes a composition comprising: a matrix comprising a hydrogel-forming polymer; and comprised in the matrix, i) a polyoxyethylated non-ionic surfactant; (ii) celecoxib; (iii) a precipitation inhibitor. The celecoxib is in an amount of at least 2%. The non-ionic surfactant may be in an amount of at least 40%, for example at least 50%; typically, the non-ionic surfactant is in such an amount and the celecoxib is in an amount of at least 2%. The composition may contain less than 10% triglyceride. All percentages in this paragraph are by weight based on the dry weight of the composition consisting of the matrix and the substances included in the matrix. Therefore the calculation of the percentage values excludes any coating on the composition.

It will be appreciated from the preceding disclosure that it is a preferred feature of the compositions of the invention that (a) the celecoxib is in amount of at least 2% and/or (b) the non-ionic surfactant may be in an amount of at least 40%, for example at least 45% or particularly at least 50%, and/or (c) composition may contain less than 10% triglyceride. Indeed, the total glyceride content may be less than 10%. Some compositions possess a single one of features (a), (b) and (c) mentioned in this paragraph whilst other compositions possess a combination selected from:

(a)+(b)

(a)+(c)

(b)+(c)

(a)+(b)+(c).

All percentages in this paragraph and the next one are by weight based on the dry weight of the composition and, in the case of solid or dried compositions, the percentages are calculated excluding any coating and are therefore based on the composition consisting of the matrix and the substances included in the matrix. For the avoidance of all doubt, it is hereby stated that the content of this paragraph applies to the entirety of the disclosure of this specification.

Also to be mentioned as optional features of compositions of the invention are the following possibilities:
(i) a celecoxib content of at least 4% and a non-ionic surfactant content of at least 45%;
(ii) a celecoxib content of at least 4% and a non-ionic surfactant content of at least 50%;
(iii) a celecoxib content of at least 5% and a non-ionic surfactant content of at least 45%;
(iv) a celecoxib content of at least 5% and a non-ionic surfactant content of at least 50%;
(v) a celecoxib content of at least 5% and a non-ionic surfactant content of from 45% to 70%;
(vi) a celecoxib content of at least 5% and a non-ionic surfactant content of from 45% to 70%;
(vii) a celecoxib content of at least 5% and a non-ionic surfactant content of from 50% to 60%;
(viii) a celecoxib content of at least 5% and a non-ionic surfactant content of from 52.5% to 57.5%.
(ix a celecoxib content of from 4% to 10%, e.g. 4% to 9%, and a non-ionic surfactant content of at least 45%;
(x) a celecoxib content of from 4% to 10%, e.g. 4% to 9%, and a non-ionic surfactant content of at least 50%;
(xi) a celecoxib content of from 5% to 10%, e.g. 5% to 9%, and a non-ionic surfactant content of at least 45%;
(xii) a celecoxib content of from 5% to 10%, e.g. 5% to 9%, and a non-ionic surfactant content of at least 50%;
(xiii)a celecoxib content of from 5% to 10%, e.g. 5% to 9%, and a non-ionic surfactant content of from 45% to 70%;
(xiv) a celecoxib content of from 5% to 10%, e.g. 5% to 9%, and a non-ionic surfactant content of from 45% to 70%;
(xv) a celecoxib content of from 5% to 10%, e.g. 5% to 9%, and a non-ionic surfactant content of from 50% to 60%;
(xvi) a celecoxib content of from 5% to 10%, e.g. 5% to 9%, and a non-ionic surfactant content of from 52.5% to 57.5%
(xvii) a celecoxib content of from 5% to 7% and a non-ionic surfactant content of from 45% to 70%;
(xviii) a celecoxib content of from 5% to 7% and a non-ionic surfactant content of from 50% to 60%;
(xix) a celecoxib content of from 5% to 7% and a non-ionic surfactant content of from 52.5% to 57.5%.
   It goes without saying that each of the possibilities mentioned in this paragraph may be combined with other optional features mentioned in this specification that are not mutually exclusive, for example with possibilities disclosed herein for the amount of precipitation inhibitor. For example, each of the possibilities mentioned in this paragraph may be combined with each of the possible contents of anionic surfactant or cellulose polymer product specified in Table A later in this specification. Further, each the possible contents of anionic surfactant and cellulose polymer product specified respectively in columns A to F of Table A may additionally be combined with the content of hydrogel-forming polymer specified in the same one of columns A to F. Although it is redundant to say so, it may also be worth mentioning that each possibility mentioned in this paragraph may be combined with any of the possibilities disclosed in paragraph [0041] and [0042] below.

The invention provides in a particular implementation a composition which comprises: (i) a polyoxyethylated non-ionic surfactant; (ii) celecoxib; (iii) a precipitation inhibitor; and (iv) a hydrogel-forming polymer in which the surfactant, the precipitation inhibitor and the celecoxib are included; wherein the composition when combined with water is capable of releasing self-assembly structures (e.g. micelles) comprising the non-ionic surfactant and celecoxib. Said water may for example be in the form of gastric, intestinal or colonic fluid or a simulated form of one of them.

For all the compositions disclosed herein, at least a predominant portion of the precipitation inhibitor may be associated with the polymer. At least a predominant portion of the precipitation inhibitor may therefore be co-located with the polymer. The two components may share the same space, therefore.

For all the compositions disclosed herein, at least a predominant portion of the celecoxib may be associated with the polyoxyethylated non-ionic surfactant. At least a predominant portion of the celecoxib may therefore be co-located with the polyoxyethylated non-ionic surfactant. The two components may share the same space.

For all the compositions disclosed herein, the polyoxyethylated non-ionic surfactant may comprise celecoxib. The polyoxyethylated non-ionic surfactant may be heterogeneously dispersed throughout the hydrogel-forming polymer. The polyoxyethylated non-ionic surfactant may be distributed as inclusions in the hydrogel-forming polymer. At least a predominant portion of the celecoxib may therefore be in the inclusions.

The invention includes within its scope a composition which comprises: a hydrogel-forming polymer; self-assembly structures (e.g. micelles) comprising a polyoxyethylated non-ionic surfactant dispersed in the polymer; and celecoxib comprised in the self-assembly structures. The hydrogel-forming polymer may be combined with water in a gel state or in a sol state, or the hydrogel-forming polymer may be dry. may be coated.

The non-liquid compositions of the invention may be moulded and/or shaped e.g. in the form of mini minibeads e.g. spherical minibeads, or other shaped units. It will be understood that the term “spherical” refers to minibeads which seem substantially or generally of spherical shape to the human eye and does not require a sphere to a mathematical standard. In other words, “spherical” minibeads as described herein are generally spheroidal in the sense of resembling or approximating to a sphere. A population of minibeads of the disclosure, though, may contain occasional non-spheroidal minibeads resulting from the manufacturing process, and reference herein to e.g. a multiplicity of minibeads or a population of minibeads encompasses such collections of minibeads which include not only spherical (spheroidal) minibeads as described herein but also non-spherical (i.e. non-spheroidal) minibeads.

The term “released” in relation to the self-assembly structures (e.g. micelles) means free to move, egress, coalesce, dissolve, (re)emulsify etc. although actual movement, egression, coalescence, association or (re)emulsification is not a requirement i.e. may not occur and indeed may intentionally be constrained e.g. by presence of a coat or coating and/or by incorporation of certain constraining or retarding substances into the hydrogel-forming polymer matrix.

The term “self-assembly structure” refers to any type of micelle, vesicle, micellar solution, lyotropic phase, laminar or other self-organised structure that forms spontaneously in the presence of an aqueous environment, or combination thereof. As is known, such self-assembly structures form when a self-assembly structure-forming substance, e.g. comprising or consisting of a surfactant, is present above a certain critical concentration. The term includes, for example, micelles, inverted micelles and liposomes, and combinations thereof. The self-assembly structures referred to in this specification may comprise, or be, micelles. More information on self-assembly structures can be found in “Dynamics of Surfactant Self-assemblies Micelles, Microemulsions, Vesicles and Lyotropic Phases” by Raoul Zana, particularly Chapter 1, all of which is incorporated herein by reference. The release of self-assembly structures from a minibead or other composition may be determined by contacting the composition with water and observing for such structures using a suitable analytical method such as dynamic light scattering.

In certain embodiments the self-assembly structures are present as a micellar solution or a dried micellar solution. For example a micellar solution may be formed during the preparation of the composition according to the invention upon mixing of the non-ionic surfactant together with an aqueous phase comprising the hydrogel-forming polymer. A micellar solution may also be formed upon release or formation of self-assembly structures from the composition when the composition is exposed to a dissolution medium, suitably an aqueous medium, for example a gastro-intestinal fluid following oral administration of the composition.

The size of the self-assembly structures present in the composition, or which are formed and/or released when the composition is exposed to an aqueous dissolution medium may be measured using known techniques such as photon correlation spectroscopy, dynamic light scattering or NMR techniques. The optical isotropicity of a composition, for example a micellar solution, may be determined using optical methods such as polarisation microscopy. These and other methods for the characterisation of micellar solutions are well known (see for example Narang et al Int J. Pharmaceutics 345 (2007) 9-25). A particular method for measuring the self-assembly structures (including for example micelles) is dynamic light scattering.

Considering now the non-ionic surfactant, it may comprise, as well as a poly(oxyethylene) moiety, a hydrophobic chain selected from alkyl and alkenyl chains; the hydrophobic chain may be substituted, for example mono-substituted, by e.g. hydroxy, provided that its hydrophobic character is maintained. In certain embodiments the non-ionic surfactant is selected from the group consisting of: macrogol esters (also known an polyoxyl esters); macrogol ethers (also known an polyoxyl ethers); and combinations thereof. Preferably the surfactant is chosen from macrogol esters. The polyoxyl moiety may be polyoxyl 15.

The surfactant may have a high hydrophobic-lipophilic balance (HLB), for example the surfactant has a HLB value of 10 or above, for example from 10 to 15, suitably from 10 to 14, for example from 12 to 14.

The surfactant may have a wax-like character. The surfactant may comprise polyglycol esters of fatty acids, for example polyglycol mono- and di-esters of fatty acids (for example stearic acid and/or 12-hydroxystearic acid). In one embodiment the fatty acid is saturated (for example stearic acid and/or 12-hydroxystearic acid). In another embodiment the fatty acid is unsaturated or a mixture of saturated and unsaturated fatty acids. In particular, the surfactant may comprise poly(oxyethylene) esters of a fatty acid, suitably a saturated fatty acid and particularly a hydroxylated saturated fatty acid (for example stearic acid and/or 12-hydroxystearic acid). In such embodiments, a small part of the 12-hydroxy group can be etherified with polyethylene glycol. The surfactant may also comprise free polyethylene glycol.

In particular, the surfactant is for example the macrogol ester macrogol-15-hydroxystearate. A representative macrogol-15-hydroxystearate is marketed as Kolliphor® HS 15 by BASF, which conforms to the requirements of the European Pharmacopoeia monograph number 2052 Macrogol-15-hydroxystearat, published in the 6^{th} Edition, July 2006. A particular class of surfactants useful in the invention are therefore those which conform to the requirements of the European Pharmacopoeia monograph number 2052 Macrogol-15-hydroxystearat, published in the 6^{th} Edition, July 2006. Reference herein to “Kolliphor” includes reference to Kolliphor HS 15. Kolliphor HS 15 may be replaced by another surfactant meeting the requirements of said monograph number 2052.

The hydrogel-forming polymer may be a thermotropic hydrogel-forming polymer, or a combination of such polymers. In such a combination, the gelatin may predominate, i.e. consititute over 50% of the combination by weight, for example over 70%. The hydrogel-forming polymer may be gelatin.

The invention includes the following process and compositions obtainable by (having the characteristics of a composition obtained by) the process, whether directly or indirectly. The process comprises mixing:
i) a celecoxib solution comprising, or consisting essentially of, a polyoxyethylated non-ionic surfactant and celecoxib; and
ii) an aqueous solution comprising, or consisting essentially of, water, a hydrogel-forming polymer, a precipitation inhibitor and optionally a plasticiser;
   wherein the two solutions are mixed to form a clear liquid. The celecoxib may be in an amount of at least 2% by weight of the clear liquid, calculated on the basis of the dry weight of the clear liquid. . The non-ionic surfactant may be in an amount of at least 40% by weight of the clear liquid, e.g. at least 50%, calculated on the basis of the dry weight of the clear liquid.

The process of the preceding paragraph may further comprise ejecting the clear liquid through a single orifice nozzle to form droplets, the hydrogel-forming polymer then being caused or allowed to solidify whereby the droplets form minibeads. The hydrogel-forming polymer may be a thermotropic polymer or a mixture of thermotropic polymers, and the aqueous premix may in this case be at an elevated temperature.

The invention further provides a process which comprises:

(i) mixing materials comprising, or consisting essentially of, water, a hydrogel-forming polymer, an optional plasticiser, a polyoxyethylated non-ionic surfactant, a precipitation inhibitor, and celecoxib to form a micellar solution, the process optionally further comprising

(ii) formulating the micellar solution into a suitable form, e.g. a minibead, by ejecting it through a single orifice nozzle to form droplets which are caused or allowed solidify, for example wherein the polymer is a thermotropic polymer and the micellar solution is caused or allowed to pass into a cooling medium, e.g. a water-immiscible cooling liquid, in which the droplets cool to form shaped units e.g. minibeads.

The invention includes shaped units (e.g. minibeads) obtainable by the process, whether directly or indirectly, i.e. includes shaped units (e.g. minibeads) having the characteristics of shaped units obtained by the process, whether directly or indirectly

In embodiments, the invention includes a process for manufacturing a composition or dispersion of the invention which comprises: forming an aqueous premix which comprises, or consists essentially of, water, the precipitation inhibitor, the hydrogel-forming polymer and an optional plasticiser, and forming a surfactant premix which comprises, or consists essentially of, the polyoxyethylated non-ionic surfactant and celecoxib, and combining the two premixes to form a clear liquid. The clear liquid may then be formed into a minibead as described in the preceding paragraph. More particularly the manufacture of the composition may optionally comprise:

(i) forming an aqueous phase premix comprising, or usually consisting of, a solution in water of water-soluble constituents, e.g. the hydrogel-forming polymer, the precipitation inhibitor and, where present, a plasticiser (see below) as well as, in some embodiments, other consitituent(s) that are soluble in water;

(ii) forming a surfactant phase premix comprising, or usually consisting of, a solution in the non-ionic surfactant of celecoxib and, in some embodiments, other consitituent(s) having limited solubility in water;

(iii) mixing the two phases to form a clear liquid; and optionally

(iv) formulating the clear liquid into a minibead or other shaped unit, e.g. ejecting it through a single orifice nozzle to form droplets which are caused or allowed to fall into a water immiscible cooling liquid in which the droplets cool to form minibeads, and then separating the minibeads from the cooling liquid. This step (iv) itself forms part of the subject matter of the invention.

The minibeads which have been separated from the cooling liquid may be centrifuged to remove excess oil and then may be dried, particularly air-dried, e.g. at ambient temperature (say 15-30° C., e.g. 20-25° C.). The centrifuging normally takes place before the drying.

Further provided by the invention is a process which comprises mixing a polyoxyethylated non-ionic surfactant and celecoxib to form a solution. The resultant surfactant mix may be mixed with an aqueous phase premix as described above. The invention therefore includes within its scope a product (e.g. a composition of matter, a solution) that comprises, or is, a mixture of celecoxib and a polyoxyethylated non-ionic surfactant. The surfactant and the weight ratio of the polyoxyethylated non-ionic surfactant to celecoxib may be as described elsewhere herein. The surfactant and the celecoxib may be in relative amounts as described elsewhere herein.

Another process of the invention is a process which comprises mixing materials comprising, or consisting essentially of, (i) water, (ii) a hydrogel-forming polymer, (iii) a polyoxyethylated non-ionic surfactant, (iv) celecoxib, (v) a precipitation inhibitor, and optionally (vi) a plasticiser to form a micellar solution.

The invention includes a micellar solution having a continuous phase comprising water, a hydrogel-forming polymer and a precipitation inhibitor, and an internal phase comprising celecoxib and a polyoxyethylated non-ionic surfactant.

The invention further provides a product having the characteristics of a composition obtained by drying a clear liquid or micellar solution as described herein.

The compositions described herein may be free or substantially free from hydrophobic molecules other than celecoxib and any other hydrophobic drug that is contained in the composition. The compositions described herein may be free or substantially free from oils. The compositions described herein may be free or substantially free from lipids. The compositions described herein may be free or substantially free from lipids and hydrocarbons. As to the meaning of “free from”, please see below under the heading “Impurities”.

The compositions described herein may be free or substantially free from triglycerides; an example of such a glyceride is Miglyol® 810. The compositions described herein may be free or substantially free from glycerides.

The compositions described herein may contain less than 10%, less than 5%, less than 1%, less than 0.5%, less than 0.1% or less than 0.001% by weight, calculated on the dry weight of the composition, of triglycerides. The compositions described herein may contain less than 10%, less than 5%, less than 1%, less than 0.5%, less than 0.1% or less than 0.001% by weight, calculated on the dry weight of the composition, of glycerides. The compositions described herein may contain less than 10%, less than 5%, less than 1%, less than 0.5%, less than 0.1% or less than 0.001% by weight, calculated on the dry weight of the composition, of oils. The compositions described herein may contain less than 10%, less than 5%, less than 1%, less than 0.5%, less than 0.1% or less than 0.001% by weight, calculated on the dry weight of the composition, of hydrophobic molecules selected from lipids and hydrocarbons, and combinations thereof. The compositions described herein may contain less than 10%, less than 5%, less than 1%, less than 0.5%, less than 0.1% or less than 0.001% by weight, calculated on the dry weight of the composition, of hydrophobic molecules other than celecoxib and any other hydrophobic drug that is contained in the composition.

As described in more detail later in this specification, a minibead made as described herein may after its preparation be coated with one or more layers. Therefore, a minibead may be dried and then coated.

There are next listed various optional features of the products (e.g. compositions, formulations and dosage forms) described herein. For the avoidance of all doubt, it is hereby stated that the content of this paragraph applies to the entirety of the disclosure of this specification. In this listing and throughout the disclosure of this specification unless otherwise required, percentages are by weight calculated on dry weight of the composition excluding coatings and excluding any additional unit dosage capsules or containers. In other words for a minibead the percentages are calculated on the dry weight of the composition obtainable by formulating a clear liquid into an uncoated minibead, excluding therefore any coatings on the minibead in calculating the percentage. The optional features of which one or more may be possessed by the products of the disclosure are as follows:
(i) the product comprises at most 5% by weight of 2-(2-ethoxyethoxy)ethanol;
(ii) the product comprises at most 1% by weight of 2-(2-ethoxyethoxy)ethanol;
(iii) the product is free of 2-(2-ethoxyethoxy)ethanol;
(iv) (the product comprises at most 5% of hydrophobic molecules other than celecoxib;
(v) the product comprises at most 1% of hydrophobic molecules other than celecoxib;
(vi) the product is free of hydrophobic molecules other than celecoxib;
(vii) the product comprises at most 5% of triglycerides;
(viii) the product comprises at most 1% of triglycerides;
(ix) the product is free of triglycerides;
(x) the product comprises at most 5% of glycerides;
(xi) the product comprises at most 1% of glycerides;
(xii) the product is free of glycerides;
(xiii) the product comprises at most 5% of lipids;
(xiv) the product comprises at most 1% of lipids;
(xv) the product is free of lipids;
(xvi) the aggregate content of lipids and hydrocarbons is at most 5%;
(xvii) the aggregate content of lipids and hydrocarbons is at most 1%;
(xviii)the product is free of lipids and hydrocarbons;
(xix) the product comprises feature (i) and further includes at least a single one of features (iv) to (xix);
(xx) the product comprises feature (ii) and further includes at least a single one of features (iv) to (xix)
(xxi) the product comprises feature (iii) and further includes at least a single one of features (iv) to (xix).

There are next listed other optional features of the products (e.g. compositions, formulations and dosage forms) described herein, of which one or more may be possessed by the products of the disclosure, optionally together with other possible features mentioned in this specification:
(a) the polyoxyethylated non-ionic surfactant is in an amount of at least 40%;
(b) the polyoxyethylated non-ionic surfactant is in an amount of at least 45%;
(c) the polyoxyethylated non-ionic surfactant is in an amount of at least 50%;
(d) the polyoxyethylated non-ionic surfactant is in an amount from 40% to 70%;
(e) the polyoxyethylated non-ionic surfactant is in an amount from 50% to 70%;
(f) the polyoxyethylated non-ionic surfactant is in an amount from 50% to 60%;
(g) the polyoxyethylated non-ionic surfactant is in an amount from 52.5% to 57.5%
(h) the product comprises feature (a) and further includes at least a single one of features (i) to (xxi) of the preceding paragraph;
(i) the product comprises feature (b) and further includes at least a single one of features (i) to (xxi) of the preceding paragraph;
(j) the product comprises feature (c) and further includes at least a single one of features (i) to (xxi) of the preceding paragraph;
(k) the product comprises feature (d) and further includes at least a single one of features (i) to (xxi) of the preceding paragraph;
(l) the product comprises feature (f) and further includes at least a single one of features (i) to (xxi) of the preceding paragraph;

(m) the product comprises feature (f) and further includes at least a single one of features (i) to (xxi) of the preceding paragraph;
(n) the product comprises feature (g) and further includes at least a single one of features (i) to (xxi) of the preceding paragraph.

In a further aspect, the present invention provides for a dosage form comprising a population of optionally coated minibeads of the invention. The dosage form is suitable for pharmaceutical use. In certain embodiments the dosage form may comprise at least two populations of minibeads, of which at least one population consists of minibeads of the invention. All the populations of minibeads may be as described herein but with differing release profiles, e.g. comprising different coating(s). At least a single population of minibeads may differ from the minibeads described herein and, in particular, may be free of celecoxib but comprise one or more other drugs.

In certain embodiments the dosage form comprises the composition (e.g. a minibead or shaped unit and particularly multiple minibeads or shaped units) of the invention in a unit dosage form suitable for administration, for example to a human or animal. The unit dosage form may be chosen from a capsule, a tablet, a sprinkle, a sachet, a suppository, a pessary or other suitable unit dosage form.

In a representative embodiment a dosage form of the invention is formed by mixing together at least the following materials to form a clear liquid: water, a hydrogel-forming polymer, celecoxib, a polyoxyethylated non-ionic surfactant, a precipitation inhibitor and optionally a plasticiser, and formulating the liquid into a dosage form (suitable for pharmaceutical use) comprising a minibead which comprises the clear liquid in a dry state. The clear liquid may be a micellar solution

In some embodiments the dosage form has been appropriately formulated in such a way as to release the celecoxib and optionally at least one other active ingredient at one or more specified locations in the gastrointestinal tract (GIT). In particular the dosage form is formulated to release the celecoxib in at least the colon, for example the celecoxib may be released in the ileum and colon or may substantially be released exclusively in the colon. The dosage form may comprise minibeads coated with a pH-dependent or pH-independent coating to target release at least partially in the colon, for example the dosage form may be a capsule or other format comprising a plurality of coated minibeads.

At least normally, the dosage forms of the invention are for enteral administration. The dosage forms of the invention are in particular for oral administration. The dosage forms of the invention may, however, be for rectal administration.

The invention includes a method for administering celecoxib to a subject, comprising enterally, e.g. orally, administering to the subject a composition or dosage form as disclosed herein).

The invention also includes a method for administering celecoxib to a subject, the method comprising enterally administering a dosage form comprising a population of minibeads as described herein. The dosage form may be for oral administration. The administration may be in the therapeutic and/or prophylactic treatment of a disease or condition.

Provided by the invention also is a product having the characteristics of a composition obtained by drying a clear liquid as described herein and the use of the product in the manufacture of an enteral, e.g. oral, dosage form, for example a gelatin capsule comprising multiple minibeads. The clear liquid may be a micellar solution.

Included in the invention is a solid composition comprising celecoxib contained in a polymer matrix, the celecoxib being solubilised in a polyoxyethylated non-ionic surfactant, the polymer matrix being associated with a precipitation inhibitor and the polymer being a hydrogel-forming polymer. The solid composition may be a minibead.

Also provided by the invention is a method for performing a therapeutic or prophylactic treatment of a disease or medical condition described herein, for example a treatment selected from:
i. treating colorectal cancer;
ii. treating familial adenomatous polyposis (FAP), including without limitation variants such as, for example, attenuated familial adenomatous polyposis;
iii. treating non-malignant polyps;
iv. treating inflammation;
v. treating an inflammatory disease;
vi. killing colorectal cancer cells in a patient, in which method apoptosis is favoured over necrosis; and
vii. inhibiting, reducing or delaying metastasis of a colorectal cancer,
   the method comprising administering a composition or dosage form as disclosed herein. The inflammation may be colonic inflammation and the inflammatory disease may be an inflammatory bowel disease, for example Crohn's disease or ulcerative colitis.

Also provided by the invention is a composition or dosage form as disclosed herein, wherein the composition or dosage form is for use in the therapeutic or prophylactic treatment of a disease or medical condition described herein, for example a treatment selected from:
i.treating colorectal cancer;
ii.treating familial adenomatous polyposis (FAP), including without limitation variants such as, for example, attenuated familial adenomatous polyposis;
iii. treating non-malignant polyps;
iv. treating inflammation;
v. treating an inflammatory disease;
vi. killing colorectal cancer cells in a patient, in which method apoptosis is favoured over necrosis; and
vii. inhibiting, reducing or delaying metastasis of a colorectal cancer.

The present invention provides compositions, formulations and dosage form that have the potential to reduce the unwanted cardiovascular and/or gastrointestinal side effects associated with the marketed celecoxib product, Celebrex®, for example by using at least one of the following strategies: a) presenting the drug in a soluble format to address its solubility issues thereby allowing for the use of a lower dose; b) incorporating the drug in a multiparticulate format thereby allowing for better GIT distribution and less likelihood of local irritation (Tang E S K et al. Am J Drug Deliv 2005; 3(1):17-28.); c) delaying release to allow for the development of colon-targeted minibeads that would enable local action on diseased tissue and thereby again reduce the dose required. The presentation of the celecoxib in solubilised form in a polyoxyethylated non-ionic solvent permits target cells to be contacted by the drug in an active form, and may facilitate administration of celecoxib in a low dosage at which it is active yet produces reduced side-effects as compared to commercially available celecoxib medicament. The normal site of drug absorption for systemic uptake is the small intestine, in particular the duodenum and the jejenum, and systemic side effects may be at least mitigated by targeting release wholly or partially on the ileum and/or colon.

Throughout the description and paragraphs of this specification, the words “comprise” and “contain” and variations of them mean “including but not limited to”, and they are not intended to (and do not) exclude other moieties, additives, components, integers or steps. Throughout the description and paragraphs of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying paragraphs, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying paragraphs, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### BRIEF DESCRIPTION OF THE DRAWINGS

FIG. 1 A plot showing percentage of drug released from dissolution testing of Formulation A, Formulation B, Formulation C, Celebrex® and celecoxib API (all tested in purified water). The percentage of celecoxib release was significantly different (p<0.01) for Formulations A, B and C when compared to Celebrex® (n=3). The data presented are mean values±standard deviation and ANOVA (analysis of variance) was used for variance analysis.

FIG. 2 Photographs of representative minibeads from Formulation D.

FIG. 3 Photographs of representative minibeads from Formulation E demonstrating the “tailing effect”.

FIG. 4 A plot of percentage of drug released from dissolution testing of Formulation G, Formulation H, and Celebrex®, all tested in purified water. The percentage of celecoxib release was significantly different (p<0.01) for Formulations G and H when compared to Celebrex® (n=3). The data presented are mean values ± standard deviation and ANOVA was used for variance analysis.

FIG. 5 Photographs of three formulations: (A) 10× image of gelatin formulation, (B) 10× image of Formulation H and (C) 10× image of Formulation G. The discontinuities shown in FIG. 5(C) are artefacts of the photographic process.

FIG. 6 The effect of celecoxib on the proliferation and viability of HT29 cells. (A) HT29 cells were plated in 96-well plates and after 24 h the cells were treated with celecoxib (celecoxib liquid formulations A and S Celebrex® and celecoxib dissolved in molecular grade DMSO) at 20, 30, 50 and 100 μM (n=6 for each concentration) for 72 h. Cellular proliferation was determined by MTT assay. (B), (C), (D), (E) HT29 cells were plated in 24-well plates 24 h prior to treatment with celecoxib at 50μM (celecoxib liquid formulations A and S Celebrex® and celecoxib dissolved in molecular grade DMSO). Seventy two hours later, cells were double stained with recombinant Annexin V-fluorochrome PE conjugate and 7-AAD and survival profiles monitored by flow cytometry. Viable cells (Annexin V⁻ and 7-AAD⁻), necrotic cells (Annexin V⁻ and 7-AAD⁺), early apoptotic cells (Annexin V⁺ and 7-AAD⁻) and late apoptotic cells (Annexin V⁺ and 7-AAD⁺) were plotted as a percentage of the total population for each treatment (n =6). Data for 10,000 events was collected for each replicate.

FIG. 7 The effect of celecoxib on the motility of HT29 cells. (A) Scratch-wound healing assay was conducted and inverted microscope images are shown immediately after the wound (0 h) and after treatment with celecoxib at 50 μM (celecoxib liquid formulations A and S, Celebrex® and celecoxib dissolved in molecular grade DMSO) (n=9 for each group) for 72 h. Images for the control group are also shown. (B) The histogram shows the percentage wound closure the control and for the celecoxib treatments 72 h after the wound persisted.

FIG. 8 Percentage of drug released from dissolution testing of formulation A, formulation S, Celebrex and celecoxib API (all tested in purified water). The percentage of celecoxib release was significantly different (p<0.01) for Formulations A and S when compared to Celebrex® and celecoxib API (dissolved in DMSO) (n=3). The data presented are mean values±standard deviation..

FIG. 9 Percentage of drug released from dissolution testing of formulation G1, formulation G2 and Celebrex® tested in two step enteric media (0.1 M HCl for 2 hrs followed by Phosphate Buffer (pH 6.8) for 22hrs). The data presented are mean values±standard deviation.

FIG. 10 Percentage of drug released from dissolution testing of formulation G3, formulation G4, formulation G5 and formulation G6 tested in two step enteric media (0.1 M HCl for 2 hrs followed by Phosphate Buffer (pH 6.8) for 22 hrs). The data presented are mean values±standard deviation.

FIG. 11 Percentage of drug released from dissolution testing of formulation G5 at 1% SDS, 0.5% SDS and 0% SDS in the dissolution media (pH 6.8). The data presented are mean values ± standard deviation.

### DETAILED DESCRIPTION

The term “clear” means clear to the eye.

The term “associated with” includes reference to two substances being mixed or co-located with each other. In the invention, the celecoxib may be associated with the non-ionic surfactant. The precipitation inhibitor may be associated with the hydrogel-forming polymer. It may be true both that the celecoxib is associated with the non-ionic surfactant and that the precipitation inhibitor is associated with the hydrogel-forming polymer. Converseley, the precipitation inhibitor may at least predominantly not be associated with the non-ionic surfactant. Similarly, the celecoxib may at least predominantly not be associated with the hydrogel-forming polymer. In implementations, the precipitation inhibitor is at least predominantly not be associated with the non-ionic surfactant, and the celecoxib is at least predominantly not associated with the hydrogel-forming polymer. Such association may be determined by identifying co-location of the adjuvant and the active using a suitable analytical technique. (Co-location means the existence of at least one location in which both substances are located). Similarly, co-location of a precipitation inhibitor and a hydrogel-forming polymer may be identified to show association between the two. Analytical techniques to determine co-location may include Time-of Flight Secondary Ion Mass Spectrometry (ToFSIMS), mapping Raman spectroscopy and infrared spectroscopy. ToFSIMS is a standard analytical technique in which a solid sample is bombarded with a primary beam of ions. Secondary ions are emitted from the sample surface and these are mass analysed to provide detailed surface chemical information; elements, chemical groups, molecules, polymer groups etc. This information can then be processed into visual images for each component detected with brighter areas indicating higher quantities of the particular component.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the appropriate governmental agency or listed in the U.S. Pharmacopoeia or other generally recognized pharmacopoeia for use in animals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “release”, particularly in relation to self-assembly structure (e.g. micelles), includes reference both to releasing pre-existing self-assembly structures in a polymer matrix and to release self-assembly structures comprising surfactant not in self-assembly structure form in the polymer matrix but formed after ingestion of a composition of the invention as water and the self-assembly structure come into mutual contact. In other words a self-assembly structure released from a composition of the invention may be preformed in the composition or formed (in whole or in part) as part of the release process.

The term “treating” includes: (1) inhibiting or delaying the appearance of clinical symptoms of the state, disorder or condition developing in an animal that may be afflicted with or predisposed to the state, disorder or condition but does not yet experience or display clinical or subclinical symptoms of the state, disorder or condition; (2) inhibiting the state, disorder or condition (e.g., inhibiting, reducing or delaying the development of the disease, or a relapse thereof in case of maintenance treatment, or of at least one clinical or subclinical symptom thereof); and/or (3) relieving the condition (i.e., causing regression of the state, disorder or condition or at least one of its clinical or subclinical symptoms). A treatment may prevent occurrence (e.g. re-occurrence) of a condition, delay occurrence of the condition, reduce the incidence of the condition, arrest initiation or development of the condition, or cause regression of the condition. A treatment may inhibit, reduce or delay initiation and/or development of a condition, or reverse the development of the condition. The benefit to a patient to be treated may be either statistically significant or at least perceptible to the patient or to the physician. It will be understood that a medicament will not necessarily produce a clinical effect in every patient to whom it is administered, and this paragraph is to be understood accordingly. The terms “state”, “disorder” and “condition” as used herein include without limitation reference to a state, disorder or condition that is associated with an increased risk of colorectal cancer as compared to the risk of a normal population. In particular, the state, order or condition treated in accordance with the invention is colorectal cancer; the cancer may be treated prophylactically in a subject who has not experienced it but is at increased risk of suffering from it, for example because the subject suffers at least intermittently, e.g. continuously, from an inflammatory bowel disease, polyps or familial adenomatous polyposis (FAP). Alternatively, the colorectal cancer may be treated therapeutically or prophylactically in a subject who suffers from colorectal cancer, in particular to arrest, inhibit, reduce or delay progression, e.g. metastasis or growth, of such cancer, or to cause its regression. The term “therapeutic or prophylactic” encompasses the same subject matter as previously mentioned in this paragraph.

The term “subject” includes birds, humans and other mammals, for example domestic animals (e.g., dogs and cats). The term “subject” in particular denotes a human.

“Effective amount” means an amount sufficient to result in the desired therapeutic or prophylactic response. The therapeutic or prophylactic response can be any response that a user (e.g., a clinician) will recognize as an effective response to the therapy. It is further within the skill of one of ordinary skill in the art to determine appropriate treatment duration, appropriate doses, and any potential combination treatments, based upon an evaluation of therapeutic or prophylactic response.

The terms “dry” and “dried” as applied to compositions of the disclosure may each include reference to compositions containing less than 5% free water by weight, e.g. less than 1% free water by weight. Primarily, however, “dry” and “dried” as applied to compositions of the disclosure mean that the hydrogel present in the initial solidified composition has, if required, dried sufficiently to form a rigid composition.

The term “percent” (%), unless otherwise stated or required, means percent by weight based on the dry weight of the composition. In the case of solid or dried compositions, the percentages are calculated excluding any coating and are therefore based on the composition consisting of the matrix and the substances included in the matrix.

The term “predominant portion” means “more than half”, for example a predominant portion may be at least 60%, at least 70%, at least 80% at least 90% or at least 95%. Where the specification refers to “at least a predominant portion” of a substance, the term includes reference to the possibility to all, or substantially all, the substance being in that “portion”. The predominant portion may be calculated by weight in appropriate instances.

The term “hydrophobic molecule” refers in particular to a molecule that is no more than sparingly soluble in water. Such hydrophobic molecules may be characterised by a positive Gibbs energy of transfer when being transferred from a nonpolar solvent to water. In addition, the hydrophobicity of a molecule may be quantified by the measurement of the partition constant. The partition constant (log P) is a measure of a ratio of concentrations of compounds in water and a second non-aqueous solvent such as octanol.

log P_{OW}=log([solute]_{octanol}/[solute]_{water})

Increasingly negative log P_{OW} values indicate increasingly hydrophilic compounds, whereas increasingly positive log P_{OW} values indicate increasingly hydrophobic compounds. As such, a hydrophobic molecule may be defined as a molecule having a positive value of logP_{ow} where the solvent is octanol. For the purposes of this invention, a hydrophobic molecule may have a positive logP_{ow}, for example a value of at least 1, at least 2, at least 3, at least 4, at least 5 or at least 6. In particular, a hydrophobic molecule may have a logP_{ow} of at least 6.

The following standard solubility definitons are reproduced for convenience:

| | Volume (ml) |
|---|---|
| Description | of Solvent Needed to Dissolve 1 g of Solute |
| Very Soluble | Less than 1 |
| Freely Soluble | 1 to 10 |
| Soluble | 10 to 30 |
| Sparingly Soluble | 30 to 100 |
| Slightly Soluble | 100 to 1000 |
| Very Slightly Soluble | 1000 to 10,000 |
| Practically Insoluble | Greater than 10,000 |

As previously described the invention provides amongst other things a composition that is a clear liquid comprising water, a hydrogel-forming polymer, celecoxib, a polyoxyethylated non-ionic surfactant and a precipitation inhibitor, and a composition, e.g. a minibead, obtainable by drying such a composition. The solid composition, e.g. minibead, is pharmaceutically useful, in particular for treating colorectal diseases, for example colorectal cancer.

The invention will now be described in detail by reference to the uses of the invention and the various components which the composition of the invention may comprise. The term “excipient” may be used occasionally to describe all or some of the components other than the active ingredient(s) bearing in mind that some excipients can be active and that some active principles can have excipient character.

If not otherwise stated, ingredients, components, excipients etc of the composition of the invention are suitable for one or more of the intended purposes discussed elsewhere herein e.g. are pharmaceutically acceptable.

For the avoidance of doubt, it is hereby stated that the information disclosed earlier in this specification under the heading “Background” is relevant to the invention and is to be read as part of the disclosure of the invention.

### Use

The invention provides pharmaceutical products comprising celecoxib, as described in more detail elsewhere in this specification. More particularly, the invention provides, for pharmaceutical use, a solid composition comprising celecoxib contained in a polymer matrix. The celecoxib may be solubilised in a polyoxyethylated non-ionic surfactant. The products of the invention are in particular minibeads and multiple minibead formulations.

The products of the invention are in particular for use in treating diseases of the colon and/or rectum and adapted to release celecoxib at least in the colon. The products may for example be adapted to release the celecoxib in the ileum and the colon, or they may be adapted to release substantially all the celecoxib in the colon. The products may therefore be colon-targeted.

The products of the invention that are adapted to release celecoxib at least in the colon may be for use in treating colorectal cancer by way of therapy or prophylaxis. The colorectal cancer may be an adenocarcinoma. The colorectal cancer may be a COX-2 positive cancer. The colorectal cancer may be a COX-2 positive adenocarcinoma. The products of the invention may be for use in treating a patient who has colorectal cancer or for use in treating a patient assessed as being at greater risk of colorectal cancer than the general population; for example the patient may have a genetic or familial predisposition to a colorectal cancer or may have a condition that increases the risk of a colorectal cancer. The condition that increases the risk of a colorectal cancer may be an inflammatory bowel disease (“IBD”), for example colitis, in particular ulcerative colitis, diverticulitis or Crohn's disease. IBD patients have a 20-fold higher risk of developing colorectal cancer than the general population. The condition that increases the risk of a colorectal cancer may be the occurrence of one or more polyps, whether or not the polyp or polyps have been surgically removed, for example the condition may be familial adenomatous polyposis (FAP), including without limitation variants such as, for example, attenuated familial adenomatous polyposis.

Prophylaxis may be primary, e.g. to prevent, reduce the risk of, delay or inhibit a first occurrence of colorectal cancer. Prophylaxis may be secondary, e.g. to prevent, reduce the risk of, delay or inhibit a re-occurrence of colorectal cancer. The prophylaxis may be to arrest, inhibit, reduce the risk of, or delay initiation or occurrence of a colorectal cancer.

The therapy may be to treat a patient who is suffering from a colorectal cancer. The therapy may be to relieve the cancer, for example to cause regression of the cancer or at least one of its clinical or subclinical symptoms. The therapy may be to inhibit, reduce or delay growth of a tumour, e.g. several tumours. The therapy may be to inhibit, reduce or delay metastasis of a colorectal cancer, e.g. of a primary tumour.

The products of the invention that are adapted to release celecoxib at least in the colon may be for use in treating inflammation of the colon. The treatment may be by way of therapy or prophylaxis. The products may therefore be for use in treating an inflammatory bowel disease, for example colitis particularly ulcerative colitis, diverticulitis or Crohn's disease. The inflammatory bowel disease may be selected from diversion colitis, ischemic colitis, infectious colitis, chemical colitis, microscopic colitis (including collagenous colitis and lymphocytic colitis), atypical colitis, pseudomembranous colitis, fulminant colitis, autistic enterocolitis, indeterminate colitis, ileocolitis, Crohn's (granulomatous) colitis, irritable bowel syndrome, mucositis, radiation induced enteritis, diverticulitis, pouchitis, proctitis, and chronic diarrhea.The products of the invention may be for use in treating a patient who has an inflammatory bowel disease, for example one mentioned in this specification, or who suffers from inflammation of the colon, or for use in treating a patient assessed as being at greater risk of inflammatory bowel disease and/or colorectal cancer than the general population; for example the patient may have a genetic or familial predisposition to inflammatory bowel disease.

The products of the invention may be used generally for the prophylactic or therapeutic treatment of gastrointestinal inflammatory conditions. In the case of each condition, the product will release the celecoxib along one or more sites of the GI tract appropriate to expose to celecoxib the gastrointestinal region that suffers from, or is at increased risk of suffering from, a gastrointestinal inflammatory condition. The products of the invention may therefore be used for the prophylactic or therapeutic treatment of one or more conditions selected from: inflammatory bowel diseases, diversion colitis, ischemic colitis, infectious colitis, chemical colitis, microscopic colitis (including collagenous colitis and lymphocytic colitis), atypical colitis, pseudomembranous colitis, fulminant colitis, autistic enterocolitis, indeterminate colitis, Behcet's disease, gastroduodenal CD, jejunoileitis, ileitis, ileocolitis, Crohn's (granulomatous) colitis, irritable bowel syndrome, mucositis, radiation induced enteritis, short bowel syndrome, celiac disease, stomach ulcers, diverticulitis, pouchitis, proctitis, and chronic diarrhea. The products of the invention may therefore be used for the treatment of patients who suffer from, or at are increased risk of suffering from, a condition mentioned in this paragraph.

In some instances, the products may be used to treat inflammatory intestinal diseases, inflammatory bowel disease, celiac disease, Crohn's disease, ulcerative colitis, GI-GVHD, gastroenteritis, duodenitis, jejunitis, ileitis, peptic ulcer, Curling's ulcer, appendicitis, colitis, pseudomembraneous colitis, irritable bowel syndrome e.g. irritable bowel syndrome—diarrhea predominant (IBS-D), irritable bowel syndrome—constipation predominant (IBS-C) and irritable bowel syndrome—mixed (IBS-M); diverticulosis, diverticulitis and endometriosis.

The products may be used to treat enteropathies, for example gluten-sensitive enteropathy, hemorrhagic enteropathy, protein-losing enteropathy, radiation enteropathy, HIV-enteropathy, enteropathy associated with T-cell lymphoma, autoimmune enteropathy or porcine proliferative enteropathy.

The treatments may include maintenance therapy of patients who have suffered a GI tract disorder and whose condition has subsequently improved, e.g. because of treatment. Such patients may or may not suffer a symptomatic GIT disorder. Maintenance therapy aims to arrest, reduce or delay (re-)occurrence or progression of a GIT disorder.

The invention primarily concerns the treatment of humans but other warm-blooded animals, e.g. mammals are also embraced by the invention, for example agricultural mammals and domesticated mammals. Examples are pigs, dogs and cats. For example, the products may be used to treat porcine proliferative enteropathy.

The products of the invention may be used to treat subjects that have a condition or disease mentioned in this specification, that have suffered a condition or disease mentioned in this specification and/or that have been diagnosed as being at enhanced risk of suffering a condition or disease mentioned in this specification.

Aspirin and other NSAIDS may be used to reduce the risk of developing gastrointestinal polyps and gastrointestinal cancers. However, a significant number of individuals are intolerant to aspirin and/or other NSAIDS. The National Institute for Health and Care Excellence (NICE) in the UK, formerly called the National Institute for Health and Clinical Excellence, have defined aspirin intolerance as either a proven hypersensitivity to aspirin, or a history of severe indigestion caused by lowdose aspirin [National Institute for Health and Clinical Excellence, 2005]. The prevalence of aspirin intolerance is between 6% and 20% with ‘true’ aspirin hypersensitivity occurring in 0.6-2.4% of the general population [Pfaar and Kilmek, 2006; Steg, 2005]. Aspirin and other nonsteroidal anti-inflammatory drugs (NSAIDs) are contraindicated in patients with a history of hypersensitivity including asthma, angioedema, urticaria, or rhinitis. The products of the invention may be administered to patients who suffer from aspirin intolerance or having a history of aspirin hypersensitivity, in particular hypersensitivity to aspirin for the prophylactic or therapeutic treatment of an inflammatory condition, for example an inflammatory condition of the gastrointestinal tract, the condition optionally being one mentioned in this specification. The products of the invention may be administered to patients undergoing aspirin desensitisation therapy. The products of the invention may be administered to patients who suffer from intolerance to an NSAID other than celecoxib or having a history of hypersensitivity to an NSAID other than celecoxib, in particular such an NSAID for the prophylactic or therapeutic treatment of an inflammatory condition.

The products of the invention may administer celecoxib in a combination therapy with another drug selected from anti-inflammatory drugs, immunosuppressants and anti-cancer drugs (e.g. chemopreventive and/or chemotherapeutic drugs), and combinations thereof. Such a combination therapy may provide an improved clinical response, for example it may improve response to the other drug(s) and/or the celecoxib treatment with the product of the invention may act as an adjuvant to other therapies. The products of the invention may administer celecoxib concurrently, therefore, with another drug as mentioned in this paragraph. The drugs administered in a combination therapy (concurrently) may be administered simultaneously, sequentially or separately. The minibeads or other compositions of the invention may contain both celecoxib and at least one other drug as mentioned herein. The oral dosage forms of the invention may contain both celecoxib and at least one other drug as mentioned herein, for example a multiple minibead formulation (e.g. capsule) may comprise celecoxib in minibeads of the invention and another drug elsewhere in the formulation, whether in a second population of minibeads, in a liquid vehicle containing also the minibeads of the invention, or in a powder contained in the formulation or otherwise.. Alternatively, at least one other drug used in combination or concurrent therapy with celecoxib may be administered in a separate dosage form from the celecoxib, e.g. a separate capsule or tablet.

Prophylaxis may be primary, e.g. to prevent, reduce the risk of, delay or inhibit a first occurrence of an inflammatory bowel disease. Prophylaxis may be secondary, e.g. to prevent, reduce the risk of, delay or inhibit a re-occurrence of an inflammatory bowel disease. The prophylaxis may be to arrest, inhibit, reduce the risk of, or delay initiation or occurrence of an inflammatory bowel disease.

The therapy may be to treat a patient who is suffering from an inflammatory bowel disease. The therapy may be relieve the inflammatory bowel disease, for example to cause regression of the disease or at least one of its clinical or subclinical symptoms. The therapy may be to inhibit, reduce or delay development of the inflammatory bowel disease.

A product of the invention will be adapted to release the celecoxib at a suitable region or regions of the GI tract for the intended treatment, e.g. to release the celecoxib in at least the colon for treating a disease or condition affecting the colon. In general, a product of the invention for treating a disease or condition affecting one or more regions of the GI tract will be adapted to release celecoxib in the region or regions concerned. Such adaptation, when appropriate, may be achieved by a controlled release coating.

### Impurities

All chemical products contain impurities and the products mentioned in this specification are no exception. Most of the constituents mentioned in the specification are natural products or are derived from natural products, and the commercially available substances are mixtures in most cases. For example, SDS normally contains alkyl sulfate homologues whilst polyoxyl-15-hydroxystearate is presumably derived from fatty acid glycerides and may contain trace amounts of glycerol and glycerides. Celecoxib API contains small amounts of impurities.

The constituents mentioned in the specification are therefore to be understood to include those that are commercially available in terms of impurities and minor ingredients. Therefore, when it is stated that a composition is free from a certain chemical or class of chemicals, it is still permitted to contain that chemical or class of chemicals at an impurity level, in particular at an impurity level corresponding to impurity amounts of the constituents as commercially available. Thus, the expression “free from glycerides” permits trace or impurity amounts of glyceride to be present, corresponding to glyceride present as a minor amount in the constituents of the composition, for example the non-ionic surfactant and/or the polymer.

### Active Ingredient

The compositions of the invention comprise celecoxib as an active ingredient. The celecoxib may be, and often is, the sole active ingredient. Alternatively, at least one further active ingredient may be included. Where the compositions are made by combining an aqueous premix and a non-ionic surfactant premix, a water-soluble further active (further drug) may be included in, especially dissolved in, the aqueous premix whilst a water-insoluble further drug (i.e. one of very limited solubility in water) may be included in, especially dissolved in, the non-ionic surfactant premix together with the celecoxib. Active ingredients that may be used in combination therapy with celecoxib are described elsewhere in this specication.

### Polymer Matrix

The disclosure includes minibeads and other solid formulations comprising a matrix to include the celecoxib and provide mechanical strength. The matrix comprises a hydrogel-forming polymer. Such formulations therefore comprise a polymer matrix.

A hydrogel-forming polymer is a polymer capable of forming a hydrogel. A hydrogel may be described as a solid or semi-solid material, which exhibits no flow when at rest, comprising a network (matrix) of hydrophilic polymer chains that span the volume of an aqueous liquid medium.

The composition may comprise a hydrogel-forming polymer selected from the group consisting of: gelatin; agar; agarose; pectin; carrageenan; chitosan; alginate; starch; xanthan gum; gum Arabic; guar gum; locust bean gum; polyurethane; polyether polyurethane; cellulose; cellulose ester, cellulose acetate, cellulose triacetate; cross-bonded polyvinyl alcohol; polymers and copolymers of acrylic acid, hydroxyalkyl acrylates, hydroxyethyl acrylate, diethylene glycol monoacrylate, 2-hydroxypropylacrylate, 3-hydroxypropyl acrylate; polymers and copolymers of methacrylic acid, hydroxyethyl methacrylate, diethyleneglycol monomethacrylate, 2-hydroxypropyl methacrylate, 3-hydroxypropyl methacrylate, dipropylene glycol monomethylacrylate; vinylpyrrolidone; acrylamide polymers and copolymers, N-methylacrylamide, N-propylacrylamide; methacrylamide polymers and copolymers, N-isopropyl methacrylamide, N-2-hydroxyethylmethacrylamide; and vinyl pyrrolidone; and combinations thereof. In specific embodiments binary or tertiary etc combinations of any of the above substances are foreseen.

The hydrogel-forming polymer may also be referred to as a hydrocolloid i.e. a colloid system wherein the colloid particles are dispersed in water and the quantity of water available allows for the formation of a gel. In embodiments it is preferred to use reversible hydrocolloids preferably thermo-reversible hydrocolloids (e.g. agar, agarose, gelatin etc) as opposed to irreversible (single-state) hydrocolloids. Thermo-reversible hydrocolloids can exist in a gel and sol state, and alternate between states with the addition or elimination of heat. Gelatin, agar and agarose are thermo-reversible, rehydratable colloids and are particularly preferred. Gelatin derivatives such as, for example, succinated or phthalated gelatins are also contemplated. Thermoreversible hydrocolloids which may be used according to the invention include those derived from natural sources such as, for example, carrageenan (extracted from seaweed), gelatin (extracted from bovine, porcine, fish or vegetal sources), agar (from seaweed), agarose (a polysaccharide obtained from agar) and pectin (extracted from citrus peel, apple and other fruits). A non-animal based hydrocolloid may be preferred for certain applications e.g. administration to vegetarians or to individuals not wishing to ingest animal products for religious or health reasons. In relation to the use of carrageenan, reference is made to U.S. patent application Ser. No. 2006/0029660 A1 (Fonkwe et al), the entirety of which is incorporated herein by reference. The hydrogel-forming polymer may comprise or be a combination of gelatin with one or more other thermoreversible hydrocolloids, e.g. with one or more other of the thermoreversible hydrocolloids just listed. The hydrogel-forming polymer may comprise or be a combination of gelatin with agar; optionally, at least one further thermoreversible hydrocolloid may be included in the combination, for example one just listed. Where the polymer comprises a mixture that includes gelatin, the gelatin may predominate, i.e. be more than 50% by weight of the mixture, for example more than 70% by weight of the mixture.

Thermo-reversible colloids present a benefit over other hydrogel-forming polymers. Gelation or hardening of thermo-reversible colloids occurs by cooling the colloid, e.g. in a liquid cooling bath or by air flow. Gelation of other hydrogel-forming polymers, which is chemically driven, can lead to leakage of the composition contents into the gelation medium as the hardening process can take time to occur. Leakage of the content of the composition may lead to an inaccurate quantity of the active ingredient within the composition. Thermo-reversible colloids are also known as thermo-reversible gels, and it is therefore preferred that the hydrogel former be a thermo-reversible gelling agent.

Another term which may be applied to hydrogel formers which are advantageous is “thermotropic”: a thermotropic gelling agent (which the reader will infer is preferred as a hydrogel former used in the invention) is one caused to gel by a change in temperature and such gelling agents are able to gel more rapidly than those whose gelling is chemically induced, e.g. ionotropic gelling agents whose gelling is induced by ions, for example chitosan. In embodiments of the invention, therefore, the hydrogel former is a thermotropic gel-forming polymer or a combination of such polymers.

The polymer is in particular gelatin, therefore.

The manufacture of the composition may require that the hydrogel-forming polymer be present as a solution, which is preferably an aqueous solution. The hydrogel-forming polymer, e.g. gelatin, represents from 5% to 50%, preferably from 10% to 30%, still more preferably from 15% to 25% or, in particular, 15% to 20% by weight of the aqueous phase during manufacture. The amount of the hydrogel-forming polymer relative to the amount of water may be from 15 to 30% by weight of the weight of water, e.g. 20 to 25%

The composition may comprise at least 25% by weight of the hydrogel-forming polymer, suitably at least 28% by weight, e.g. at least 30% by weight, based upon the dry weight of the composition. The maximum content of the polymer in the composition may be 40% or 37.5%, e.g. 35% or 32%, the percentages being by weight, based upon the dry weight of the composition. For example the hydrogel-forming polymer is present in an amount of from 25 to 40%, for example 28 to 40%, suitably 25 to 37.5% or 28 to 37.5%, of the composition, wherein the % is by weight based upon the dry weight of the composition. In particular, such percentage content of the polymer may be from 25 to 35% or 28 to 35%, e.g. 30 to 35% or 28 to 32%.

The hydrogel-forming polymer is of course a pharmaceutically acceptable polymer.

In certain embodiments the hydrogel-forming polymer is gelatin. In certain embodiments the hydrogel-forming polymer comprises gelatin. In certain embodiments the gelatin comprises at least 25%, for example 28 to 35% and suitably 31 to 32% of the composition, wherein the % is by weight based upon the dry weight of the composition.

In embodiments in which the polymer comprises or is gelatin, reference is hereby made to “Bloom strength”, a measure of the strength of a gel or gelatin developed in 1925 by O. T. Bloom. The test determines the weight (in grams) needed by a probe (normally with a diameter of 0.5 inch) to deflect the surface of the gel 4 mm without breaking it. The result is expressed in Bloom (grades) and usually ranges between 30 and 300 Bloom. To perform the Bloom test on gelatin, a 6.67% gelatin solution is kept for 17-18 hours at 10° C. prior to being tested.

When the hydrogel-forming polymer comprises or is gelatin the bloom strength of the gelatin may be in the range of 125 Bloom to 300 Bloom, 200 Bloom to 300 Bloom and preferably 250 Bloom to 300 Bloom. It should be appreciated that higher bloom strength gelatin can be replaced by lower bloom strength gelatin at higher concentrations.

According to the invention, in embodiments in which the water-soluble polymer matrix material comprises or is gelatin, the gelatin may be sourced by a variety of means. For example, it can be obtained by the partial hydrolysis of collagenous material, such as the skin, white connective tissues, or bones of animals. Type A gelatin is derived mainly from porcine skins by acid processing, and exhibits an isoelectric point between pH 7 and pH 9, while Type B gelatin is derived from alkaline processing of bones and animal (bovine) skins and exhibits an isoelectric point between pH 4.7 and pH 5.2. Type A gelatin is somewhat preferred. Gelatin for use in the invention may also be derived from the skin of cold water fish. Blends of Type A and Type B gelatins can be used in the invention to obtain a gelatin with the requisite viscosity and bloom strength characteristics for minibead manufacture.

Lower temperature gelatin (or gelatin derivatives or mixtures of gelatins with melting point reducers) or other polymer matrices able to be solidified at lower temperatures (e.g. sodium alginate) are preferred for example when an active agent to be incorporated in the composition of the invention is temperature-labile or whose activity may be affected by exposure to higher temperatures. A polymer which comprises or is low temperature gelatin is a possible matrix polymer in this invention.

Where the polymer comprises or is gelatin, the starting gelatin material is preferably modified before manufacture to produce “soft gelatin” by the addition of a plasticizer or softener to the gelatin to adjust the hardness of the composition of the invention. The addition of plasticizer achieves enhanced softness and flexibility as may be desirable to optimise dissolution and/or further processing such as, for example, coating. Useful plasticizers of the present invention for combination with gelatin or another hydrogel-forming polymer include glycerine (1,2,3-propanetriol), D-sorbitol (D-glucitol), sorbitol BP (a non-crystallizing sorbitol solution) or an aqueous solution of D-sorbitol, sorbitans (e.g. Andidriborb 85/70), mannitol, maltitol, gum arabic, triethyl citrate, tri-n-butyl citrate, dibutylsebacate. Other or similar low molecular weight polyols are also contemplated for example ethylene glycol and propylene glycol. Polyethylene glycol and polypropylene glycol may also be used although these are less preferred. Glycerine and D-sorbitol may be obtained from the Sigma Chemical Company, St. Louis, Mo. USA or Roquette, France. Therefore, the hydrogel-forming polymer may optionally comprise, or be associated with, a plasticiser, for example at least one polyol. The plasticiser may comprise, or be, at least one of glycerol, sorbitol and a sorbitol/sorbitan mixture, and may comprise, or be, sorbitol or glycerol, or a combination thereof. The plasticiser may be sorbitol. In particular one or more plasticisers as mentioned in the paragraph may be combined with gelatin, and therefore associated with gelatin in the compositions.

Softeners or plasticisers, if utilized, can be ideally incorporated in a proportion rising to 10% by dry weight of the composition, for example to 8% by dry weight of the composition, e.g. from 2% to 8%, and optionally from 4% to 6%. In particular, the plasticiser content may be from 3% to 4% by dry weight of the composition.

Although not essential, the hydrogel-forming polymer may also optionally contain a disintegrant where it is particularly desired to enhance the rate of disintegration of the composition of the invention. Examples of disintegrants which may be included are alginic acid, croscarmellose sodium, crospovidone, low-substituted hydroxypropyl cellulose and sodium starch glycolate.

The polymer matrix may be, or comprise, chitosan which can exist in the form of biogels with or without additives as described e.g. in U. S. Pat. No. 4,659,700 (Johnson & Johnson); by Kumar Majeti N. V. Ravi in Reactive and Functional Polymers, 46, 1, 2000; and by Paul et al. in ST.P. Pharma Science, 10, 5, 2000 the entirety of all 3 of which is incorporated herein by reference. Chitosan derivatives e.g. thiolated entities are also contemplated.

The hydrogel-forming polymer of the end product composition, e.g. minicapsule, may have a low water content, therefore the end product composition, e.g. minicapsule, may have a low water content.

In certain embodiments the composition does not comprise compounds containing a disulphide bond. In embodiments the hydrogel-forming polymer does not comprise compounds containing a disulphide bond.

### Precipitation Inhibitor

The composition may comprise a precipitation inhibitor. Where the compositions are made by combining an aqueous premix and a non-ionic surfactant premix, the precipitation inhibitor may be included in, especially dissolved in, the aqueous premix. In the end product composition, e.g. minibead, at least a predominant proportion of the precipitation inhibitor, e.g. substantially all of it, may be associated with, or be in, the polymer. Accordingly, the precipitation inhibitor may at least predominantly share the same space as the hydrogel-forming polymer.

As previously indicated, it is believed (without being bound by theory) that the clear liquid of the invention is a micellar solution and that the solidified composition is therefore a dried micellar solution. Accordingly, an aspect of the invention is a composition comprising celecoxib, a polyoxyethylated non-ionic surfactant, a precipitation inhibitor, and a hydrogel-forming polymer in which the celecoxib and the surfactants are included, wherein the composition has a feature selected from:
(i) when combined with water, the composition is capable of releasing self-assembly structures comprising non-ionic surfactant and celecoxib;
(ii) the polymer forms a matrix in which the polyoxyethylated non-ionic surfactant is distributed as inclusions that comprise celecoxib and have a size of less than 25μm.
   When such a composition having the feature (i) comprises a precipitation inhibitor, the precipitation inhibitor may be at least predominantly outside the inclusions. When such a composition having the feature (ii) comprises a precipitation inhibitor, it may be that the precipitation inhibitor at least predominantly does not share the same volumes occupied by the non-ionic surfactant.

The precipitation inhibitor may therefore be partitioned between the aqueous and the non-ionic surfactant phases of a micellar solution such that it is at least predominantly in the aqueous phase. Correspondingly, where the composition has the characteristics of a dried micellar solution, the non-ionic surfactant may be present as inclusions within the polymer matrix and the precipitation inhibitor is at least predominantly outside the inclusions. The composition when combined with water may be capable of releasing self-assembly structures comprising non-ionic surfactant and celecoxib.

The precipitation inhibitor may therefore be partitioned between the aqueous and the non-ionic surfactant phases of a micellar solution such that it at least predominantly does not share the same volumes occupied by the non-ionic surfactant. Correspondingly, where the composition has the characteristics of a dried micellar solution, the polymer may form a matrix in which the polyoxyethylated non-ionic surfactant is distributed as inclusions that comprise celecoxib, e.g.having a size of less than 25 μm, the precipitation inhibitor being at least predominantly outside the inclusions.

The precipitation inhibitor may be selected from, or comprise, anionic surfactants and cellulose polymers, and combinations thereof. The precipitation inhibitor may in general comprise a combination of inhibitors.

As an anionic surfactant may be mentioned sulfates and sulfonates, e.g. aliphatic sulfates and aliphatic or aromatic sulfonates and combinations thereof, for example alkyl sulfates. A particular alkyl sulfate is dodecyl sulfate. The sulfates and sulfonates may be ammonium or alkali earth metal salts, for example alkali metal salts and, in particular sodium salts. Accordingly, the anionic surfactant may be sodium dodecyl sulfate (SDS). The anionic surfactant may comprise, or be, a substance (whether or not a mixture) as mentioned in this paragraph. The precipitation inhibitor, therefore, may comprise, or be, a substance (whether or not a mixture) as mentioned in this paragraph. Further to be mentioned as anionic surfactants for inclusion in the aqueous phase include perfluoro-octanoate (PFOA or PFO), perfluoro-octanesulfonate (PFOS), ammonium dodecyl sulfate (SDS), sodium laureth sulfate also known as sodium lauryl ether sulfate (SLES) and alkyl benzene sulphonate. Mixtures of anionic surfactants are also contemplated.

As a cellulose polymer may be mentioned hydroxypropyl cellulose and hydroxypropylmethyl cellulose, and a combination of hydroxypropyl cellulose and hydroxypropylmethyl cellulose. The cellulose polymer, and therefore the precipitation inhibitor may comprise, or be, hydroxypropyl cellulose or hydroxypropylmethyl cellulose, or a combination thereof. A commercially available product that comprises hydroxypropylmethyl cellulose (“HPMC”) is Opadry White 20A28380 (Opadry White is a trade mark), and this may be used as the precipitation inhibitor, either alone or in combination with one or more other precipitation inhibitors. The ingredients of Opadry® White 20A28380 are talc, hydroxypropyl cellulose, hydroxypropylmethyl cellulose (also called hypromellose) and titanium dioxide. The precipitation inhibitor may comprise or be HPC and/or HPMC (e.g. a combination of both), optionally together with (i) TiO₂ and/or (ii) talc, e.g. with both TiO₂ and talc. The precipitation inhibitor may comprise or be a combination of talc, hydroxypropyl cellulose, hydroxypropylmethyl cellulose (also called hypromellose) and titanium dioxide. Optionally, at least one further precipitation inhibitor may be incorporated in addition to those mentioned in this paragraph.

The precipitation inhibitor may be or comprise: a vitamin E compound, for example vitamin E TPGS (D-α-tocopheryl polyethylene glycol succinate) or vitamin E acetate; PVA (polyvinyl alcohol), HPMC, in particular having a viscosity of 10-20 mPa s, e.g. 15 mPa s, for a 2% solution in water at 20° C. (e.g. as available under the trade mark Methocel E15). Such materials may cause difficulties in processing (see Example 6) and be less preferred.

### Polyoxyethylated Non-Ionic Surfactant

The polyoxyethylated non-ionic surfactant is a surfactant that comprises a poly(oxyethylene) moiety.

The surfactant of course has a hydrophobic part and in particular a hydrophobic chain. The hydrophobic chain may be a hydrocarbon chain, for example having at least 6 carbon atoms and optionally at least 10 carbon atoms, and particularly of at least 12 carbon atoms; some hydrocarbon chains have no more than 22 carbon atoms, for example C₁₀-C₂₀, C₁₂-C₂₀ or C₁₅-C₂₀ hydrocarbon chains. It may be an alkyl chain, e.g. having a number of carbon atoms just mentioned. It may be an alkenyl chain comprising one or more carbon-carbon double bonds, e.g. having a number of carbon atoms just mentioned. The surfactant may comprise a hydrocarbon chain, e.g. alkyl chain or alkenyl chain, that is substituted provided that it maintains a hydrophobic characteristic. There may for example be one or two substituents, for example a single substituent, e.g. selected from halogen (e.g. F or Cl), hydroxy, thiol, oxo, nitro, cyano; hydroxy or thiol substituents may be esterified by for example a fatty acid.

The hydrophobic chain may be part of an esterified fatty acid R¹—COOH or of an etherified or esterified fatty alcohol R¹—COH where R¹ is, or comprises, the hydrophobic chain, e.g. as mentioned in the preceding paragraph. The ester-forming or, as the case may be, ether-forming group will comprise, or be, a poly(oxyethylene) chain. A portion of the fatty acid molecules R¹—COOH or fatty alcohol molecules R¹—COH may be as the free acid or alcohol and a portion may be esterified or, in the case of fatty alcohols, etherified with a group that comprises, or is, a poly(oxyethylene) chain.

The poly(oxyethylene) chain is also known as a poly(ethyleneglycol) or macrogol. The poly(oxyethylene) chain may be of the formula —(O—CH₂—CH₂)ₙ—OR where n is 5 or 6 to 50 and R is H or alkyl, e.g. ethyl or methyl. The invention includes implementations in which n is from 6 to 40, e.g. from 6 to 35. In some embodiments, n is from 6 to 25 and optionally is from 8 to 25 or from 8 to 15. In other embodiments, n is from 8 to 50 or from 8 to 40, e.g. is from 10 to 50, 10 to 40 or 10 to 35. In a particular embodiment, n is 15. For all poly(oxyethylene) chains of the formula —(O—CH₂—CH₂)ₙ—OR, in one class of embodiments R is H.

The surfactant may comprise a combination of a hydrophobic chain as described above and a hydrophilic chain as described above. It may therefore be, or comprise, a macrogol ester (also known as a polyoxyl ester) of a fatty acid as described herein or a macrogol ether (also known as a polyoxyl ether) of a fatty alcohol as described herein.

Examples of macrogol esters which are suitable for use in the present invention are macrogol esters (polyoxyl esters) of fatty acids having at least 6 carbon atoms and optionally at least 10 carbon atoms, and particularly of at least 12 carbon atoms; some fatty acids have no more than 22 carbon atoms, for example C₁₀-C₂₀, C₁₂-C₂₀ or C₁₅-C₂₀ fatty acids. The fatty acids may be saturated or unsaturated but are in particular saturated. To be mentioned are macrogol 25 cetostearyl ether (Cremophor® A25); macrogol 6 cetostearyl ether (Cremophor® A6); macrogol glycerol ricinoleate 35 (Kolliphor® EL); macrogol-glycerol hydroxystearate 40 (Cremophor® RH 40); macrogol-15-hydroxystearate (polyoxyl-15-hydroxystearate US Pharmacopoeia and National Formulary, European Pharmacopoeia, e.g. Kolliphor HS 15, previously known as Solutol® HS 15). Examples of macrogol ethers which are suitable for use in the present invention are macrogol ethers of fatty alcohols having at least 6 carbon atoms and optionally at least 10 carbon atoms, and particularly of at least 12 carbon atoms; some fatty alcohols have no more than 22 carbon atoms, for example C₁₀-C₂₀, C₁₂-C₂₀ or C₁₅-C₂₀ fatty alcohols. The fatty alcohols may be saturated or unsaturated but are in one embodiment saturated. Kolliphor® HS 15 is obtained by reacting 15 moles of ethylene oxide with 1 mole of 12-hydroxy stearic acid; the surfactant may therefore be or comprise a surfactant obtainable by (having the characteristics of a surfactant obtained by) reacting 10-25 moles of ethylene oxide with 1 mole of 12-hydroxy stearic acid; the number of moles of ethylene oxide may , from 12-25 and optionally from 15-20, e.g. 15 or 20.

Kolliphor® HS 15 consists of polyglycol mono- and di-esters of 12-hydroxystearic acid and about 30% of free polyethylene glycol. The main components of the ester part have the following chemical structures:

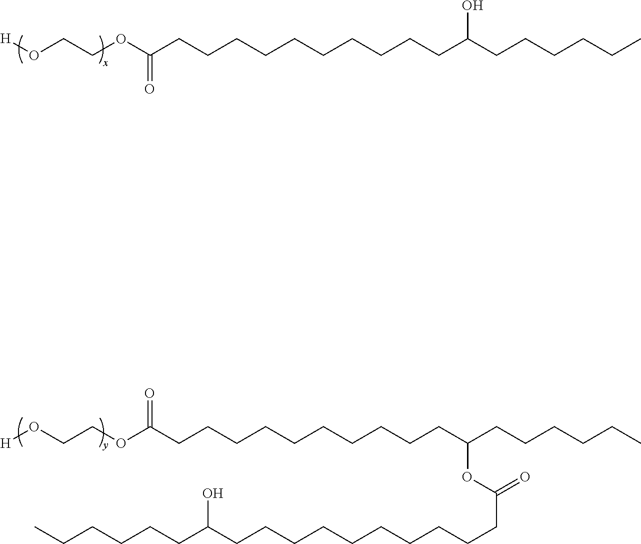

where x and y are integers and a small part of the 12-hydroxy group can be etherified with polyethylene glycol.

Therefore, the surfactant may comprise a mixture of molecules. For example, the surfactant composition used in the manufacturing process may comprise a polyethoxylated (PEGylated) molecule and comprise additionally free polyethoxy (free PEG) compound, or the surfactant composition used in the manufacturing process may comprise a molecule having a polyhydroxylated moiety and comprise additionally free polyhydroxy compound. Amongst the implementations of the invention are those in which the surfactant is, or comprises, a PEGylated fatty acid, e.g. a PEGylated hydroxy fatty acid, in combination with free PEG.

In a preferred embodiment the surfactant is a macrogol ester, more preferably a macrogol ester that conforms to the European Pharmacopoeia monograph number 2052 macrogol-15-

The non-ionic surfactant may be present in the composition in an amount of at least 40%, e.g. of at least 45% and optionally of at least 50%. It may be in an amount of from 40% to 70%, for example 45% to 70% or 45% to 67% by weight, optionally 50% to 65%, e.g. 50% to 60%, in particular 52.5% to 57.5%. The percentages in this paragraph are by weight based upon the dry weight of the composition.

The non-ionic surfactant may be present as self-assembly structures (e.g. micelles) dispersed within the hydrogel-forming polymer in a “wet” (not yet dried) composition made as an intermediate in the manufacturing process described herein. It is believed also to be present as self-assembly structures (e.g. micelles) in the dried composition. Observability of self-assembly structures, for example micelles or micelle-like structures in the wet or dried composition is not a requirement of the invention. It is mentioned at this point that the presence of a surfactant in a self-assembly structure (e.g. micelle) form does not require that the entire surfactant content of a composition is in this form as it is considered more probable that a portion of the surfactant will be outside the self-assembly structures (e.g. micelles).

### Proportions

The weight ratio of the hydrogel-forming polymer to the non-ionic surfactant may be from 1:1 to 1:3 and optionally from 1:1.3 to 1:2.6 or from 1:1.4 to 1:2.2. More particularly the weight ratio of the hydrogel-forming polymer to the polyoxyethylated non-ionic surfactant is from 1:1.5 to 1:2.1, e.g. from 1:1.5 to 1:1.9. For example, in such instances the polymer may be wholly or predominantly gelatin and the non-ionic surfactant may be a polyoxyl fatty acid, for example polyoxyl-15-hydroxystearate. In particular, the polymer is gelatin and the non-ionic surfactant is as described elsewhere in this specification, and especially is polyoxyl-15-hydroxystearate. The composition may comprise a plasticiser associated with the gelatin. The plasticiser may be sorbitol.

The weight ratio of the precipitation inhibitor to celecoxib may be from 1:0.2 to 1:10 and optionally from 1:0.8 to 1:6. Where the precipitation inhibitor is an anionic surfactant, e.g. SDS, the weight ratio of the anionic surfactant to celecoxib is optionally from 1:0.8 to 1:3, e.g. from 1:1 to 1:2.3, in particular may be from 1:1 to 1:2, for example from 1:1.2 to 1:1.6. Where the precipitation inhibitor comprises a cellulose polymer, e.g. HPC and/or HPMC, the weight ratio of the precipitation inhibitor to celecoxib may be from 1:2 to 1:8, for example from 1:3 to 1:8 or from 1:3 to 1:7, particularly from 1:3.5 to 1:5.5. The precipitation inhibitor comprising a cellulose polymer may in particular comprise HPC and HPMC optionally together with (i) TiO₂ and/or (ii) talc, e.g. with both TiO₂ and talc, and may be Opadry® White 20A28380. The weight ratio of the cellulose polymer(s) to celecoxib may be from 1:2 to 1:8, for example from 1:3 to 1:8 or from 1:3 to 1:7, particularly from 1:3.5 to 1:5.5.

The weight ratio of the polyoxyethylated non-ionic surfactant to celecoxib may be from 35:1 to 4: 1, optionally from 13:1 to 5:1. More particularly, the weight ratio of the polyoxyethylated non-ionic surfactant to celecoxib is from 7.5:1 to 11.5:1, optionally about 9:1.

The composition, excluding any coatings, may comprise, or consist essentially of, the constituents in the amounts stated in the following Table A for any of the formulations designated A to F.

As regards the “wet” compositions that contain a solution of the polymer in water, the weight ratio of the surfactant phase (non-ionic surfactant and celecoxib) to the aqueous phase (water, polymer, precipitation inhibitor, optional plasticiser) may be from 1:2 to 1:6 or 1:2 to 1:5, e.g. 1:2.5 to 1:4.5 or 1:3 to 1:4.

TABLE A

| | A | B | C | D | E | F |
|---|---|---|---|---|---|---|
| Constituent | Weight % | Weight % | Weight % | Weight % | Weight % | Weight % |
| Celecoxib | 2-10 | 2-10 | 4-10, e.g. | 4-9 | 5-9 | 5-7 |
| | | | 5-10 | | | |
| Non-ionic | 40-70 | 45-70, e.g. | 50-65 | 50-60 | 52.5-57.5 | 52.5-57.5 |
| surfactant | | 45-67 | | | | |
| Polymer | 25-40 | 25-40, e.g. | 25-37.5 | 25-35, e.g. | 28-35 | 30-35 |
| | | 28-40 | | 28-35 | | |
| Anionic | 0.5-10, | 1-5 | 1-5 | 3-5 | 3-5 | 3-5 |
| surfactant or | e.g. 1-10 | | | | | |
| Cellulose | 0.5-10, | 0.5-3 | 0.5-3 | 0.5-3 | 0.5-2 | 0.5-2 |
| polymer | e.g. 0.5-5 | | | | | |
| product | | | | | | |
| Plasticiser | 0-8, e.g. | 2-8 | 2-8 | 2-6 | 2-6, e.g. | 2-6, e.g. |
| | 2-8 | | | | 3-4 | 3-4 |

In Table A above, the following apply: the anionic surfactant and the cellulose polymer product are alternative precipitation inhibitors; the above percentages are by dry weight; in Formula B the amount of the hydrogel-forming polymer is from 28-40 when the amount of the non-ionic surfactant is from 45-67; and the total percentage contents of the constituents excluding water add up to 100. It goes without saying that the anionic surfactant may be a mixture of anionic surfactants and is in particular SDS.

The term “cellulose polymer product” means “a product comprising a cellulose polymer”. The cellulose polymer may be a mixture of cellulose polymers, the cellulose polymer in particular being, or comprising, HPMC or HPC. A particular cellulose polymer product comprises HPC and, more especially, a combination of HPMC and HPC. The cellulose polymer product may contain one or more additional ingredients to the cellulose polymer(s), for example one or more of a PEG, talc and TiO₂, e.g. the product may comprise: HPC and talc; HPC and TiO₂; or HPC, talc and TiO₂. The cellulose polymer product may be Opadry® White 20A28380, or otherwise be, for example, a combination of substances comprising or consisting of HPMC and HPC, e.g. as previously described herein. In particular, the cellulose polymer product may be a combination comprising or consisting of HPMC, HPC, talc and TiO₂. The cellulose polymer(s) may constitute more than 50% by weight of a cellulose polymer product.

### Further Information on Constituents

The non-ionic surfactant, the polymer, the precipitation inhibitor and the optional plasticiser may each individually have an identity set out in Table B below. A composition of the invention may have constituents which in combination have an identity specified in any of columns (a) to (f) in Table B. Thus, all of the the polymer, the precipitation inhibitor and the plasticiser, where present, may be as specified in any of columns (a) to (f) or merely one, two or three thereof may be as specified in any of columns (a) to (f).

TABLE B

| | (a) | (b) | (c) | (d) | (e) | (f) |
|---|---|---|---|---|---|---|
| Constituent | Identity | Identity | Identity | Identity | Identity | Identity |
| Non-ionic | Comprises a | Comprises a | Polyoxyl | Polyoxyl | Polyoxyl- | Polyoxyl-15- |
| surfactant | polyoxyethyl | polyoxy- | fatty acid | hydroxy fatty | 15- | hydroxy- |
| | ester or | ethylated | (macrogol | acid | hydroxy- | stearate |
| | ether | aliphatic acid | ester of fatty | | stearate | |
| | | | acid) | | | |
| Polymer | Thermo- | Thermo- | At least one | Gelatin; and | Gelatin | Gelatin |
| | reversible | reversible | of gelatin; | optionally a | | |
| | hydrogel- | hydrogel- | agar; | minor | | |
| | forming | forming | agarose; | proportion of | | |
| | polymer(s) | polymer(s), | pectin; | agar; agarose; | | |
| | | gelatin at least | carrageenan; | pectin; | | |
| | | predominating | chitosan. | carrageenan; | | |
| | | | | and/or | | |
| | | | | chitosan. | | |
| Precipitation | An anionic | An anionic | An aliphatic | An alkyl | A | Sodium |
| Inhibitor | surfactant or | surfactant, or | sulfate or | sulfate or | dodecyl | dodecyl |
| | a cellulose | cellulose | sulfonate, or | cellulose | sulfate or | sulfate or a |
| | polymer | polymer | cellulose | polymer | a mixture | mixture of |
| | product | product that | polymer | product that | of | HPMC, |
| | | comprises | product that | comprises | HPMC, | HPC, talc |
| | | HPC and/or | comprises | HPC and/or | HPC, talc | and TiO₂ |
| | | HPMC | HPC and/or | HPMC | and TiO₂ | |
| | | | HPMC | | | |
| Plasticiser | At least one | A polyol, e.g. | A polyol, e.g. | A polyol, e.g. | Sorbitol | Sorbitol or |
| | polyol. | at least one of | at least one | at least one of | or | another |
| | | glycerol, | of glycerol, | glycerol, | another | polyol |
| | | sorbitol, | sorbitol, | sorbitol, | polyol | |
| | | sorbitol/ | sorbitol/ | sorbitol/ | | |
| | | sorbitan | sorbitan | sorbitan | | |
| | | mixture, e.g. | mixture, e.g. | mixture, e.g. | | |
| | | sorbitol. | sorbitol. | sorbitol. | | |

The combination of features of any of rows A to F of Table A may therefore be combined with the combination of features of any of rows (a) to (f) of Table B. The term “cellulose polymer product” in Table B is explained in the text following Table A.

In one class of embodiments of Table B, the precipitation inhibitor is an anionic surfactant as specified in respective ones of columns (a) to (f). In a further class of embodiments of Table B, the precipitation inhibitor is a cellulose polymer product as specified in respective ones of columns (a) to (f). Also disclosed are embodiments in which the plasticiser is, or comprises, sorbitol, for example is sorbitol.

It will be understood that the subject matter of each cell of Table B is disclosed in its own right, independent of the context of Table B. In other words, the subject matter of each cell may be applied across the entirety of the disclosure of this specification.

### Further Excipients

The invention foresees the possible incorporation into the composition of one or more additional substances, e.g. one or more of the following substances or categories of substances in addition to the above-mentioned substances, e.g. in an amount of no more than 10%, optionally no more than 5%, e.g. no more than 3%, wherein the percentages are by weight based on the dry weight of the composition excluding any coatings. More usually, however, excluding impurities and minor constituents as mentioned above, the composition (excluding any coatings) consists of celecoxib, non-ionic surfactant, hydrogel-forming polymer, precipitation inhibitor and plasticiser, where present. The liquid composition additionally includes a significant amount of water but, in the solid composition, the amount of water is considered normally to be no more than a few percent as outlined earlier in this specification.

As examples of additional excipients, the composition may contain a protectant such as, for example, a proteolytic enzyme inhibitor or a protector against acid degradation or both (e.g. an alkali for example sodium hydroxide); an adhesive entity such as, for example, a muco- or bio-adhesive; excipients to improve solubility of active pharmaceutical compound(s); and/or a further surfactant.

The composition may further comprise excipients to enhance the therapeutic potential of active ingredients in the colon including, but not limited to absorption limiters; essential oils such as, for example, omega 3 oils; natural plant extracts such as, for example, neem; ion-exchange resins; polysaccharides such as, for example, amylose, guar gum, pectin, chitosan, inulin, cyclodextrins, chondroitin sulphate, dextrans, guar gum and locust bean gum.

The composition may further comprise an oil, e.g. may contain a single oil or a combination of oils, which may be any pharmaceutically acceptable oil. Preferably, however, the composition does not comprise an oil. Where an oil is included, the oil content is typically less than 5% by weight, e.g. no more than 3% by weight, based on the dry weight of the composition excluding any coating. Oils may be or comprise vegetable oils (e.g. neem oil), petrochemical oils, and/or volatile essential oils. The composition may comprise an oil selected from the group consisting of: poly-unsaturated fatty acids such as, for example, omega-3 oils; medium chain triglycerides; natural triglyceride-based oils which include olive oil, sesame oil, coconut oil, palm kernel oil, preferred include saturated coconut and palm kernel oil-derived caprylic and capric fatty acids and glycerine; other possible oils include linoleoyl macrogolglycerides (polyoxylglycerides) such as, for example, Labrafil (e.g. product number M2125CS by Gattefosse) and caprylocaproyl macrogolglycerides such as, for example, Labrasol by Gattefosse.

As oils may be mentioned liquid lipids, for example selected from medium chain triglyceride (MCT) compositions, the medium chain triglyceride(s) being one or more triglycerides of at least one fatty acid selected from C₆-C₁₂ fatty acids. It will be understood that commercially available MCT compositions useful in the invention are mixtures derived from natural products and usually or always contain minor amounts of compounds which are not MCTs; the term “medium chain triglyceride composition” is therefore to be interpreted to include such compositions.

Unless otherwise stated or required, all percentages and ratios are by weight, calculated on a dry basis.

The physical form of the surfactant at the point of introduction into the aqueous phase during preparation plays a role in the ease of manufacture of the composition according to the invention. As such, although liquid surfactants can be employed, it is preferred to utilize a surfactant which is in solid form (e.g. crystalline, granules or powder) at room temperature, particularly when the aqueous phase comprises gelatin.

The composition may further comprise excipients or other active pharmaceutical or other ingredients to enhance systemic bioavailability following absorption in the GIT, such as the small intestine, including efflux pump inhibitors, including, but not limited to PgP pump inhibitors, and metabolism inhibitors, including, but not limited to, cytochrome P450 3A inhibitors.

The composition may further comprise excipients to reduce systemic side effects associated with absorption in the GIT, such as the small intestine, including, but not limited to, antioxidants, such as, for example, curcuminoids, flavanoids or more specifically including curcumin, beta-carotene, α-tocopherol, ascorbate or lazaroid.

The composition may further or separately comprise antioxidants (such as, for example, ascorbic acid or BHT—butyl hydroxy toluene) taste-masking or photosensitive components or photoprotective components. Antioxidants may be incorporated in the aqueous phase (e.g. hydrophilic antioxidants) or in the surfactant phase (e.g. hydrophobic antioxidants such as, for example, vitamin E) for example up to 1% by weight, preferably between 0.01 and 0.50% by weight, more preferably between 0.10 to 0.20% by weight.

The composition may further comprise immune-enhancing nutrients such as Vitamins A/B/C/E; Carotenoids/beta-carotene and Iron, Manganese, Selenium, Zinc. Such nutrients may be present in composition, or if the composition has a coating, for example if it is the form of a minibead, the nutrients may be included in the coating.

The composition may further or separately include an adhesive to ensure that if desired the minibead of the dosage form remain, or remain for longer, in the gastric environment. minibeads according to the invention may also comprise materials facilitating or enabling floating or density reduction e.g. as a means of localising minibeads in desired GI sites. The minibead of the dosage form may also have the means to swell and/or aggregate in the stomach or other GI site.

The dosage form of the invention may comprise the excipients disclosed above. In certain embodiments any excipients present in the dosage form may not be contained within the population of the composition of the dosage form.

### Shape, Size and Geometry

The composition of the invention can be formed into a limitless number of shapes and sizes. In the section below describing the process for making the composition, various methods are given including pouring or introducing a fluid micelle dispersion into a mould where it hardens or can be caused to harden. Thus the composition can be created in whichever form is desired by creating an appropriate mould (e.g. in the shape of a disc, pill or tablet). However, it is not essential to use a mould. For example, the composition may be formed into a sheet e.g. resulting from pouring a fluid micelle dispersion onto a flat surface where it hardens or can be caused to harden.

Preferably, the composition may be in the form of spheres or spherical-like shapes made as described below. Preferably, the composition of the invention is in the form of substantially spherical, seamless minibeads. The absence of seams on the minibead surface is an advantage e.g. in further processing, for example coating, since it allows more consistent coating, flowability etc. The absence of seams on the minibeads also enhances consistency of dissolution of the minibeads.

The preferred size or diameter range of minibeads according to the invention can be chosen to avoid retention in the stomach upon oral administration of the minibeads. Larger dosage forms are retained for variable periods in the stomach and pass the pyloric sphincter only with food whereas smaller particles pass the pylorus independently of food. Selection of the appropriate size range (see below) thus makes the prediction of therapeutic effect post-dosing more accurate. Compared to a single large monolithic oral format such as, for example, a traditional compressed pill, a population of minibeads released into the GI tract (as foreseen by the dosage form of the present invention) permits greater intestinal lumen dispersion so enhancing absorption via exposure to greater epithelial area, prevents irritation (e.g. as otherwise seen with NSAIDs) and achieves greater topical coating (e.g. as may be desired for local drug effect in certain parts of the GI tract for example the colon). Reduction of residence time in the ileo-caecal junction is another potential advantage.

The composition of the invention is preferably monolithic meaning internally (i.e. cross-sectionally) homogeneous, excluding a possible thin skin of matrix material and excluding any coating layers.

The minibeads provided for by the composition of the present invention generally range in diameter from 0.5 mm to 10 mm with the upper limit preferably 5 mm, e.g. 2.5 mm. A particularly convenient upper limit is 2 mm or 1.7 mm. The lower limit can preferably be 0.7 mm or 1 mm, e.g. 1.2 mm, more preferably from 1.3 mm, most preferably from 1.4 mm. In one embodiment the diameter is from 0.5 to 5 mm or 0.5 to 2.5 mm, for example from 0.5 mm to 2 mm, from 0.7 mm to 3 mm, 0.7 mm to 1.5 mm, from 1 mm to 3 mm, 1 mm to 2 mm, 1.2 mm to 3 mm or 1.2 mm to 2 mm. The minibeads may have a diameter of no more than 2.5 mm, irrespective of their minimum size. The minibeads may have a diameter of no more than 2 mm, irrespective of their minimum size.

A minibead as described herein may have an aspect ratio of no more than 1.5, e.g. of no more than 1.3, for example of no more than 1.2 and, in particular, of from 1.1 to 1.5, 1.1 to 1.3 or, 1.1 to 1.2. A population of minibeads as described herein, e.g. at least 10 minibeads, may have an average aspect ratio of no more than 1.5, e.g. of no more than 1.3, for example of no more than 1.2 and, in particular, of from 1 to 1.5, 1 to 1.3 or 1 to 1.2. The aspect ratios mentioned in this paragraph optionally apply to coated minibeads and optionally apply to uncoated minibeads. Average aspect ratio is suitably determined for a population of minibeads, e.g. at least 10 minibeads, using a particle size analyser, for example an Eyecon™ particle characteriser of Innopharma Labs, Dublin 18, Ireland.

The minibeads of the disclosure may, therefore, have a size as disclosed in paragraph [00194] above and an aspect ratio of from 1 to 1.5. The minibeads of the disclosure may have a size as disclosed in paragraph [00194] above and an aspect ratio of no more than 1.3, for example of no more than 1.2 and, in particular, of from 1.1 to 1.5, 1.1 to 1.3 or, 1.1 to 1.2.

Bead size (diameter) may be measured by any suitable technique, for example microscopy, sieving, sedimentation, optical sensing zone method, electrical sensing zone method or laser light scattering. For the purposes of this specification, minibead size is measured by analytical sieving in accordance with USP General Test <786> Method I (USP 24—NF 18, (U.S. Pharmacopeial Convention, Rockville, Md., 2000), pp. 1965-1967).

In embodiments, minibeads of the invention are monodisperse. In other embodiments, minibeads of the invention are not monodisperse. By “monodisperse” is meant that for a population of minibeads (e. g. at least 100, more preferably at least 1000) the minibeads have a coefficient of variation (CV) of their diameters of 35% or less, optionally 25% or less, for example 15% or less, such as e.g. of 10% or less and optionally of 8% or less, e.g. 5% or less. A particular class of polymer minibeads has a CV of 25% or less. CV when referred to in this specification is defined as 100 times (standard deviation) divided by average where “average” is mean particle diameter and standard deviation is standard deviation in particle size. Such a determination of CV is performable using a sieve.

The invention includes minibeads having a CV of 35% and a mean diameter of 1 mm to 2 mm, e.g. 1.5 mm. The invention also includes minibeads having a CV of 20% and a mean diameter of 1 mm to 2 mm, e.g. 1.5 mm, as well as minibeads having a CV of 10% and a mean diameter of 1 mm to 2 mm, e.g. 1.5 mm. In one class of embodiments, 90% of minibeads have a diameter of from 0.5 mm to 2.5 mm, e.g. of from 1 mm to 2 mm.

Another possible form of the composition of the invention is as hemispherical minibeads two of which may optionally be joined at the flat face to create a single minibead with two distinct halves, each having a distinct composition, if that is desired, e.g. each containing different active principles or the same active principles but different excipients e.g. to achieve differing permeability, solubilisation or release profiles as between the two hemispheres.

The minibead provided for by the composition of the invention, may also be used as a starting point for creation of further e.g. pharmaceutical or nutraceutical forms for example by using the minibead as a nonpareil seed on which additional layers of material can be applied as is well known to a person skilled in the art e.g. of pharmaceutical science. The material of the additional layers may comprise the same or different active principle and/or the same or different excipients as are described in this document. Such variants allow differential release of the same or different active principles and facilitate inclusion of multiple fixed-dose combination products as for example discussed in connection with the popularly termed “polypill” which denotes a single pill comprising more than one active principle in a fixed dose combination, an idea of particular relevance to cardiovascular medicine.

The composition of the invention may have a coat of additional material on its outer surface. This coat may be applied in a number of ways, including drug layering, as described more particularly in the section below entitled “coating”. In one such embodiment, the composition of the invention comprises an acid e.g. included within the hydrogel-forming polymer matrix or as a liquid core in mini-capsular format and bicarbonate applied as a coat e.g. by drug layering. If the composition has a polymeric coat, e.g. to control release into the colon, the bicarbonate may optionally or additionally be included in or be absent from the coating polymer. This composition is intended to release carbon dioxide in the GI tract e.g. to reduce pain or to reduce inflammation. In a related embodiment, the core or the composition comprises an acid to enhance the solubility of active principles of various pKa (acid dissociation constant) in the small intestine or colon. Alternatively, the core or the composition comprises a base to enhance the solubility of active principles of various pKa in the stomach.

### Other Characteristics

The composition of the invention, in certain embodiments, comprises one or more elements, components, excipients, structural features, functional features or other aspects of the prior art described above.

To summarise a limited number of embodiments of the invention, the composition as described above and elsewhere herein may additionally be one or more of the following: substantially water-free, in a gel state, in a solid state, undissolved, non-powdered, formed, shaped, and not in solution.

It is preferable that the composition of the invention is essentially or substantially dry, e.g. contains less than 5%, preferably less than 1% of free water by weight. The minibeads of the composition are preferably homogeneous although processing conditions may be varied (see below) to achieve for example heterogeneity such as, for example, a harder skin and softer core with less than complete immobilization of the micelles towards the core as opposed to the surface of the minibead. Larger forms or shapes of the minibead according to the invention may particularly be engineered to embody such heterogeneity.

The low free-water content is a distinguishing feature of certain embodiments of the compositions of the present invention. The free-water content can be measured using thermogravimetic analysis (TGA), for example with commercially available instrumentation, e.g. using a TGA Q 500 of TA Q series instrument. TGA measures changes in weight in relation to a change in temperature. For example, a TGA method can comprise a temperature scan, e.g. from 20 to 400° C. at 20° C. per minute, where the moisture content is obtained from the sample weight loss at about 100 degrees Celsius.

The “solid” composition of the invention (i.e. after solidification and drying of the hydrogel in the processes described below) is suitably such that the constituents readily form micelles in at least one of, e.g. in both of, the surfactant-containing small intestine and the surfactant-limited colon.

In one embodiment, the invention allows for minibeads or other shaped units having immediate release (IR) characteristics e.g. bearing no coat, enteric-only coat or coat designed to prevent release and/or dissolution of the minibead only for a limited time or lacking a retardant in the aqueous phase. In another embodiment, the invention allows for minibeads having delayed or sustained release (SR) characteristics e.g. bearing a coat (or more than one coat) as described in more detail below, particularly in the section entitled “coating”. The invention also provides for an embodiment in which immediate release minibeads are produced in combination with a Sustained Release or Controlled Release (CR) minibeads in varying ratios of IR:SR/CR. The immediate release minibeads can be combined with a Sustained or Controlled release minibead component in the following ratios (w/w by potency) e.g. 10% Immediate Release (IR)+90% Sustained (SR)/Controlled Release (CR) minicapsules; 20% IR+80% SR/CR; 30% IR+70% SR/CR; 40% IR+60% SR/CR and 50% IR+50% SR/CR.

In embodiments, the minibeads or shaped units have an immediate release coat and, between the core made of the micelle-containing composition and the IR coat, a sub-coat to do at least one of the following, amongst others: provide mechanical strength; prevent moisture absorption; modulate release of active agent from the core; stabilise release of active agent from the core (e.g. modulate and stabilise release of active agent from the core).

In embodiments the self-assembly structures for example micelles, are characterised by the size of the self-assembly structure. A convenient method to determine micelle size is dynamic light scattering as hereinbefore described, wherein the micelle size is determined as a hydrodynamic diameter. Light scattering is a technique which can be used to determine the size distribution profile of particles in solution. When light hits particles the light scatters in all directions (Rayleigh scattering). If the light source is a laser, and thus is monochromatic and coherent, then a time-dependent fluctuation in the scattering intensity is observed. These fluctuations are due to the fact that the particles in solutions are undergoing Brownian motion and so the distance between the scattering particles in solution is constantly changing with time. The dynamic information of the particles is derived from an autocorrelation of the intensity trace recorded during the experiment which is dependent on measured time delays. At short time delays, the correlation is high because the particles do not have a chance to move to a great extent from the initial state that they were in. As the time delays become longer, the correlation starts to exponentially decay to zero, meaning that after a long time period has elapsed, there is no correlation between the scattered intensity of the initial and final states. This exponential decay is related to the motion of the particles, specifically to the diffusion coefficient. To fit the decay, numerical methods are used, based on calculations of assumed distributions. If the sample is monodisperse then the decay is simply a single exponential and the polydispersity index (PdI) would be zero or close to it; however, if the sample is polydisperse the decay can be bi-exponential (when there are two populations), or have an even more complex decay. The polydispersity index is obtained from the fitting of the decay and will reach its maximum at PdI equal one.

Other useful characterising methods for measuring the size and formation of self-assembly structures such as micelles include Small angle X-ray scattering and Diffusion Nuclear Magnetic Resonance. Such techniques are well known, see for example Oliver et al PLOS one, May 2013, Vol 8 (5), e62488 and Colafemmina et al, J. Phys Chem B 2007, 111, 7184-7193.

### Manufacturing Processes

The manufacturing processes described herein comprise mixing of liquids. Such mixing processes must be performed at temperatures at which the substances to be mixed in the liquid state are in liquid form. For example, thermoreversible gelling agents must be mixed at a temperature where they are in the liquid state, for example at a temperature of 50 to 75° C., for example 50 to 70° C., or 55-75° C., e.g. 60-70° C. and in particular embodiments about 55° C. or 65° C. in the case of mixing compositions comprising aqueous gelatin. Kolliphor HS 15 is also to be mixed in the liquid state and is suitably maintained at room temperature or slightly higher, for example maintained at a temperature of at least 30° C. for that purpose, e.g. of 35-50° C. and in particular 40° C.; where both Kolliphor HS 15 and aqueous gelatin are to be mixed, then a higher temperature, e.g. of 50-75° C., for example 55-75° C., is used at which Kolliphor HS 15 is liquid as well as aqueous gelatin.

Compositions as disclosed herein may be made by mixing materials comprising water, a hydrogel-forming polymer, a non-ionic surfactant, a precipitation inhibitor and celecoxib plus any other active ingredient(s) to form a clear liquid. Compositions as disclosed herein may be made by mixing materials comprising water, a hydrogel-forming polymer, a non-ionic surfactant, a precipitation inhibitor, and celecoxib plus any other active ingredient(s) to form a self-assembly structure dispersion within an aqueous phase comprising the hydrogel-forming polymer. The hydrogel-forming polymer is then caused or allowed to gel. Suitably, the process includes formulating or processing the clear liquid aqueous self-assembly structure dispersion into a desired form, e.g. a minibead, which forming process may comprise moulding but preferably comprises ejecting the clear liquid or aqueous micelle dispersion through a single orifice nozzle to form droplets which are caused or allowed to pass into a cooling medium, e.g. a water-immiscible cooling liquid, in which the droplets cool to form for e.g. minibeads.

The mixing of the materials may comprise mixing an aqueous premix (or aqueous phase) and a polyoxyethylated non-ionic surfactant premix (or non-ionic surfactant phase), wherein the aqueous premix comprises water and water-soluble substance(s), in particular the hydrogel-forming polymer, the precipitation inhibitor and any plasticizer, whilst the non-ionic surfactant premix comprises non-ionic surfactant and surfactant-soluble substance(s), in particular celecoxib.

The aqueous premix comprises, or usually consists of, a solution in water of water-soluble constituents. The aqueous phase may include one or more controlled release polymers. In any event, the constituents of the aqueous premix may be agitated for a period of, for example, from 1 hour to 12 hours to form the completed aqueous premix.

The surfactant phase premix comprises, or usually consists of, a solution in a polyoxyethylated non-ionic surfactant of celecoxib and any other hydrophobic and amphiphilic constituent(s). In some embodiments, the components of the surfactant premix are mixed (or otherwise agitated) for a period of, for example, 10 minutes to 3 hours to form the premix.

The two premixes may be combined and agitated, for example for a period of a few seconds to an hour, for example from 30 seconds to 1 hour, suitably 5 mins to an hour, to form a clear liquid considered to be a dispersion of self-assembly structures (e.g. micelles) in an aqueous hydrogel-forming polymer, e.g. a micellar solution. The two premixes may be combined by agitation in a mixing vessel; they may additionally or alternatively be combined in a continuous flow mixer.

Taking account of the final composition required (as described elsewhere herein), the non-ionic surfactant premix or solution and the aqueous premix or aqueous solution may be mixed in a proportion in the range 1:2-5, preferably approximately 1:3 or 1:4. In general, only gentle stirring of the components is required using a magnetic or mechanical system e.g. overhead stirrer as would be familiar to a person skilled in the art to achieve a dispersion of self-assembly structures, e.g. micelles. Continuous stirring is preferred. Mixing may also be achieved using an in-line mixing system. Any appropriate laboratory stirring apparatus or industrial scale mixer may be utilized for this purpose for example the Magnetic Stirrer (manufactured by Stuart) or Overhead Stirrer (by KNF or Fisher). It is preferred to set up the equipment in such a way as to minimise evaporation of contents such as, for example, water. In one embodiment of the process of the invention, it is preferred to utilise a closed system for stirring in order to achieve this aim. In-line mixing may be particularly suitable for closed system processing.

Both of the surfactant and aqueous phases may be clear liquids, considered to be clear solutions, prior to mixing of them.

In embodiments mixing of the aqueous and surfactant pre-mixes results in the formation of a clear liquid, for example a micellar solution, in which the aqueous phase comprising the hydrogel-forming polymer is the continuous phase. The formation of a clear liquid upon mixing of the surfactant phase and aqueous phase is generally indicative that very small self-assembly structures have formed, for example as a micellar solution.

In the embodiments where the polymer matrix substantially consists of gelatin with the addition of sorbitol, the aqueous phase of polymer matrix is prepared by adding the appropriate quantities of sorbitol (and surfactant if desired) to water, heating to approximately 50 to 75° C., for example 60-75° C. until in solution and then adding gelatin although the precise order and timing of addition is not critical. A typical “gelatin solution” comprises 8 to 25%, (for example 15-25%, preferably 17-18%) gelatin; 75%-85% (preferably 77-82%) of water plus from 1-5% (preferably 1.5 to 3%) sorbitol.

Generally, the gelatin solution (especially in the case of standard or normal gelatin) is maintained at 50 to 70° C., suitably 60° C.-70° C., to maintain it in a fluid state.

The processing temperature can however be reduced to a desirable target temperature e.g. 37° C. by use of lower melting-point gelatin (or gelatin derivatives or mixtures of gelatins with melting point reducers) or other polymer matrix material such as, for example, sodium alginate. Alternatively, temperature-labile active principles may be processed at higher temperatures by using appropriate apparatus or machinery which limits the time during which the temperature-labile active principle is in contact with the higher temperature medium. For example, if gelatin droplets are being formed by machine extrusion and immediately cooled e.g. in a cooling bath, additional appropriate inlet tubing can be used to introduce temperature-sensitive active principle into the fluid gelatin solution (and the mixture can be immediately homogenized) very shortly before ejection from a minibeading nozzle or other dropletting process such that the duration of exposure of the active principle to the higher temperature gelatin is limited so reducing the degree of any heat-dependent degradation of the active ingredient. This process may use any appropriate device such as, for example, a homogenizer, e.g. a screw homogenizer, in conjunction with an extrusion-type apparatus as described for example in WO 2008/132707 (Sigmoid Pharma) the entirety of which is incorporated herein by reference.

Generally, where the non-ionic surfactant is a liquid there is no need to heat it and the celecoxib is added at room temperature with stirring until clear. In the embodiments where the surfactant is a waxy solid such as, for example, Kolliphor HS 15 it is appropriate to heat the waxy solid, e.g. to above 30° C., to provide a liquid.

The clear liquid resulting from combining the aqueous and surfactant phases is then shaped. It may be poured or introduced into a mould or other vessel or poured onto sheets or between sheets or delivered dropwise (or extruded) into another fluid such that the polymer matrix-containing aqueous phase, on solidification, takes the form of the mould, vessel, sheet or droplet/bead intended. It is preferred to progress to mould-forming e.g. minibeading without delay.

Alternatively to moulding, specialised or customised machinery can be employed for example to create the hemispherical minibeads described above (see section above entitled “Shape, Size and Geometry”) in which the invention takes the form of hemispherical minibeads. It is possible to manufacture a single minibead made from joining two such hemispheres (i.e. a single minibead having two distinct halves) by using specialist apparatus in which two tubes through which two different emulsions are flowing, normally of circular cross section, are joined shortly before an extrusion point or nozzle (which may be vibrating) into a single dual lumen tube with a flat wall separating the two emulsion flows and which prevents the two emulsions from coming into contact until the point of extrusion. The cross-section of the joined dual-lumen tube up to the point of extrusion therefore appears as two semicircles. In operation, the two hemispherical emulsion flows combine to form a single, substantially spherical, minibead on extrusion such that normal droplets are ejected/extruded for solidification.

Solidification can occur in a variety of ways depending on the polymer of the matrix, for example by changing the temperature around the mould, vessel, sheet, droplet/bead etc or by applying a solidification fluid or hardening solution so that the moulded shape is gelled or solidified. In certain embodiments both temperature change and application of a solidifying fluid or hardening solution are employed together or simultaneously.

In the preferred embodiment in which the composition of the invention takes the form of minibeads, the minibeads may be formed for example by dropping the clear liquid dropwise into a fluid which effects solidification. The minibeads may be produced through ejection or extrusion of the liquid dispersion through a nozzle having a single orifice and optionally subject to selected vibrational frequencies and/or gravitational flowBy use of the term “dry”, it is not sought to imply that a drying step is necessary to produce the dry micelle dispersion (although this is not excluded) rather that the solid or solidified aqueous external phase is substantially free of water or free of available water. Solidification of the aqueous phase (external phase) may have arisen through various means including chemically (e.g. by cross-linking) or physically (e.g. by cooling or heating). In this respect, the term “aqueous phase” is nevertheless employed in this document to denote the external (continuous) phase of the minibead of the invention even though water, in certain embodiments, is largely absent from (or trapped within the cross-linked matrix of) the minibead of the invention. The external phase of the composition of the invention is however water-soluble and dissolves in aqueous media. In one embodiment, self-assembly structures are released when the aqueous phase dissolves or is exposed to aqueous media, irrespective of the form adopted by the micelle-forming surfactant in the solid composition.

Accordingly, in some embodiments of the invention the composition according to the invention releases self-assembly structures upon dissolution or exposure to an aqueous medium, for example a dissolution medium such as gastro-intestinal fluid following oral administration of the composition. In embodiments the self-assembly structures released from the composition (for example micelles) are about 0.5 nm to 200 nm for example from about 1 nm to 50 nm, or about 5 nm and 25 nm. The size of the self-assembly structure (eg micelle) may be determined by for example dynamic light scattering as hereinbefore defined. Suitably, the release of such self-assembly structures (eg micelles) from a composition according to the invention may be determined by placing the composition in an aqueous dissolution medium and measuring the size of the self-assembly structures released into the dissolution medium using dynamic light scattering. The dissolution medium should be such that the matrix comprising the hydrogel forming polymer is exposed to the aqueous medium to allow the formation and/or release of self-assembly structures (for example micelles) into the dissolution medium. Accordingly, when the composition is coated to delay or control the release it may be necessary to adjust for example the pH of the medium or the residence time in the dissolution medium to enable the dissolution medium to penetrate into the core comprising the hydrogel matrix and allow the formation and/or release of the self-assembly structures into the dissolution medium. For example, when a composition is coated with an enteric coating the pH of the dissolution medium may need to be increased to pH>5.5 or >6.5 to allow dissolution of the enteric coating and exposure of the core to the aqueous dissolution medium. The dissolution medium used to measure the self-assembly structures may simply be water, for example water at pH 7.2-7.3 at 37° C. After placing the composition into the dissolution medium the dissolution medium may be regularly sampled and analysed for the presence/release of self-assembly structures from the composition using for example the dynamic light scattering methods described herein.

In the case where solidification can be achieved by reducing temperature, the temperature of the solidification fluid can be adapted to achieve solidification at a desired rate. For example, when gelatin is used as the hydrogel-forming polymer, the solidification fluid is at a lower temperature than the temperature of the emulsion thus causing solidification of the polymer matrix. In this case, the solidification fluid is termed a cooling fluid.

In the case where solidification can be achieved chemically, e.g. by induction of cross-linking on exposure to a component of the solidification fluid, the concentration of such component in the solidification fluid and/or its temperature (or other characteristic or content) can be adjusted to achieve the desired rate and degree of solidification. For example, if alginate is chosen as the polymer matrix, one component of the solidification fluid may be a calcium-containing entity (such as, for example, calcium chloride) able to induce cross-linking of the alginate and consequent solidification. Alternatively, the same or similar calcium-containing entity may be included (e.g. dispersed) in the aqueous phase of the fluid emulsion prior to minibeading and triggered to induce cross-linking e.g. by applying a higher or lower pH to a solidification fluid into which droplets of emulsion fall dropwise or are introduced. Alternatively, the clear liquid may be spray-cooled, i.e. sprayed into a cooling gas to effect solidification.

In the case of gelatin or other water-soluble polymer (or polymer mixture) destined to form the immobilization matrix, it is preferred that the solidification fluid be a non-aqueous liquid (such as, for example, medium chain triglycerides, mineral oil or similar preferably with low HLB to ensure minimal wetting) which can conveniently be placed in a bath (cooling bath) to receive the droplets of micelle dispersion as they solidify to form minibeads. Use of a non-aqueous liquid allows greater flexibility in choice of the temperature at which cooling is conducted.

Where a liquid cooling bath is employed, it is generally maintained at less than 20° C., preferably maintained in the range 5-15° C., more preferably 8-12° C. when standard gelatin is used as the hydrogel-forming polymer. A triglyceride may be chosen as the cooling fluid in the cooling bath, a preferred example being Miglyol 810 from Sasol.

If gelatin or another thermotropic polymer or polymer mixture is selected as the hydrogel-forming polymer matrix, respect for appropriate temperature ranges ensures solidification of the polymer at an appropriate rate to avoid destruction e.g. of tertiary protein structure in the case where the active principle is a protein.

If alginate is selected as the polymer matrix, a typical method of making minibeads involves dropwise addition of a 3% sodium alginate solution in which oil droplets are dispersed as described above into a 4° C. crosslinking bath containing 0.1 M calcium chloride to produce calcium alginate (this method can be referred to as “diffusion setting” because the calcium is believed to diffuse into the minibeads to effect cross-linking or setting). Using a syringe pump, or Inotech machine, droplets can be generated or extruded (egg at 5 mL/h if a pump is used) through a sterile needle or other nozzle (described elsewhere herein) which can be vibrating as discussed elsewhere herein. Airflow of between 15 and 20 L/min through 4.5 mm tubing can be applied downwards over the needle to reduce droplet size if desired. Newly formed minibeads can then be stirred in the calcium chloride bath for up to an hour. If carrageenan is used as the polymer matrix both salt and reduction in temperature e.g. by dropping into cooling oil may be used to obtain solidification.

Where another ionotropic polymer is used than alginate, suitable analogous processes may be used to those described herein in relation to alginate.

Following shape-forming, moulding or minibeading, the resultant shapes or forms may be washed then dried if appropriate. In the case of minibeads solidified in a solidification fluid, an optional final step in the method of production described above therefore comprises removal of the solidified minibeads from the solidification fluid. This may be achieved e.g. by collection in a mesh basket through which the solidification fluid (e.g. medium chain triglycerides) is drained and the minibeads retained and is preferably conducted without delay e.g. as soon as the minibeads have formed or within 5, 10, 15, 20, 25 or 30 minutes of their formation. Excess solidification fluid may then be removed using a centrifuge (or other apparatus or machine adapted to remove excess fluid) followed by drying of the minibeads to remove water or free water and/or removal of some or all of any additional solvent e.g. ethanol or isopropyl alcohol used to dissolve or facilitate dissolution of the active principle in preceding steps optionally followed by washing (e.g. using ethyl acetate) and a subsequent “drying” step to remove excess solvent (e.g. ethyl acetate). Isopropyl alcohol is an example of a solvent which is preferably removed later in processing to reduce residues in the surfactant or aqueous phase. Drying can be achieved by any suitable process known in the art such as use of a drum drier with warm air at between 15° C. and 25° C., preferably around 20° C. leading to evaporation or entrainment of the water by the air. Use of gelatin as the polymer matrix (e.g. as principal constituent of the aqueous immobilisation phase) in most cases requires a drying step and for minibeads this is preferably achieved by drying in air as above described. The resultant composition (the composition of the invention) is essentially dry as described in more detail above.

It will be appreciated, therefore, that the invention includes a process for manufacturing a composition of the invention which comprises: forming an aqueous premix, particularly an aqueous solution, which comprises water and water soluble/dispersible materials (including therefore a hydrogel-forming polymer) and a surfactant premix, particularly surfactant solution, which comprises a polyoxyethylated non-ionic surfactant, celecoxib and possible other surfactant soluble/dispersible materials, and combining the two premixes to form a clear liquid. The clear liquid may then be formed into a shaped unit, for example a minibead. More particularly the manufacture of the composition may optionally comprise:

(i) forming an aqueous phase premix comprising, or usually consisting of, a solution in water of water-soluble constituents (e.g. hydrogel-forming polymer, a precipitation inhibitor and an optional plasticiser,);

(ii) forming a surfactant phase premix comprising, or usually consisting of, a solution in a surfactant of celecoxib;

(iii) mixing the two phases to form a clear liquid; and optionally

(iv) formulating the clear liquid into a minibead, e.g. ejecting it through a single orifice nozzle to form droplets which are caused or allowed to fall into a water immiscible cooling liquid in which the droplets cool to form minibeads, and then separating the minibeads from the cooling liquid.

Some manufacturing processes comprise steps (A) to (D) below or, alternatively, a manufacturing process may comprise a single one or any combination of steps (A) to (D).

(A) Exemplary Preparation of Aqueous Phase:

Aqueous phase components are added to water, e.g. purified water, under agitation e.g. sonication or stirring. The temperature is gradually increased, for example to 55-75° C. and in particular 65° C., to achieve complete dissolution of the solids. The aqueous phase components include a hydrogel forming polymer, e.g. gelatin or agar, a precipitation inhibitor, and optionally one or more other excipients, for example D-sorbitol (a plasticiser) and optionally one or more hydrophilic active ingredients in addition to the hydrophobic celecoxib in the non-ionic surfactant phase. Possible aqueous phase components are described elsewhere herein.

The gelatin may be Type A gelatin. In some less preferred implementations, the gelatin is Type B. The gelatin may have a Bloom strength of 125-300, optionally of 200-300, for example of 250-300, and in particular 275. The components of the aqueous phase may be agitated for a period of, for example, from 1 hour to 12 hours to complete preparation of the aqueous phase (aqueous premix).

(B) Exemplary Preparation of Surfactant Phase:

Surfactant phase components are added to the surfactant under agitation e.g. sonication or stirring. The temperature is gradually increased, for example in the case of the a waxy surfactant such as Kolliphor HS 15 to, usually, 35-50° C. and in particular 40° C., to achieve complete dissolution of the solids. The components of the surfactant phase are therefore usually agitated e.g. stirred until a clear solution is obtained. The components of the surfactant phase include the surfactant, for example Kolliphor® HS15, celecoxib and optionally one or more other hydrophobic active ingredients. Possible surfactant phase components are described elsewhere herein. The components of the surfactant phase may be agitated for a period of, for example, from 10 hour to 3 hours to complete preparation of the surfactant phase (surfactant premix).

(C) Exemplary Mixing of the Two Phases

The aqueous phase and the surfactant phase are mixed. The two phases may be mixed in a desired weight; for example, the weight ratio of surfactant phase to aqueous phase may be from 1:1 to 1:10, e.g. from 1:1 to 1:6 and optionally from 1:1 to 1:4 and in some cases from 1:3 to 1:4. In other embodiments the weight ratio of surfactant phase to aqueous phase may be from 1:1 to 1:3, The resulting solution is agitated, e.g. sonicated or stirred, at an elevated temperature, in particular 65° C., to achieve a homogeneous micelle dispersion, then the homogenous dispersion is formed into minibeads. In particular, the homogenous dispersion is ejected through a single orifice nozzle to form droplets which fall into a cooling medium. The nozzle is suitably vibrated to facilitate droplet formation. The nozzle may be vibrated at a frequency of 2-200 Hz and optionally 15-50 Hz.

The cooling medium may for example be air or an oil; the oil is suitably physiologically acceptable as, for example, in the case of medium chain triglycerides e.g. Miglyol 810N. The cooling medium may be at a cooling temperature often of less than 15° C., for example of less than 10° C. but above 0° C. In some embodiments the cooling temperature is 8-10° C. The nozzle size (diameter) is typically from 0.5 to 7.5mm, e.g. from 0.5 to 5 mm and optionally from 0.5 to 4 mm. In some embodiments, the nozzle diameter is from 1 to 5 mm for example from 2 to 5 mm, and optionally from 1 to 3 mm or from 3 to4 mm. The nozzle diameter may be from 0.5 to 2 mm, e.g. from 0.5 to 1.5 mm, and optionally 1 mm.

In particular embodiments of the processes described above and elsewhere in the description, mixing of the surfactant phase and the aqueous phase results in the formation of a micellar solution or a composition having the characteristics of a micellar solution, wherein self-assembly structures comprising the surfactant are dispersed in the aqueous phase comprising the hydrogel forming polymer as hereinbefore described to provide a clear thermodynamically stable composition. For example, mixing of the two phases may result in a micellar solution comprising micelles comprising a macrogol-15-hydroxystearate, such as for example Kolliphor HS 15 dispersed in the aqueous phase comprising the hydrogel-forming polymer such as gelatin.

(D) Exemplary Processing of Minibeads

Cooled minibeads are recovered, for example they may be recovered from cooling oil after a residence time of 15-60 minutes, for example after approximately 30 minutes. Minibeads recovered from a cooling liquid (e.g. oil) may be centrifuged to eliminate excess cooling liquid, and then dried. Suitably, drying is carried out at room temperature, for example from 15-25° C. and optionally from 20-25° C. The drying may be performed in a drum drier, for example for a period from 6 to 24 hours, e.g. of about 12 hours in the case of minibeads dried at room temperature. The dried minibeads may be washed, suitably with a volatile non-aqueous liquid at least partially miscible with water, e.g. they may be washed with ethyl acetate. The washed minibeads may be dried at room temperature, for example from 15-25° C. and optionally from 20-25° C. The drying may be performed in a drum drier, for example for a period from 6 to 48 hours, e.g. of about 24 hours in the case of minibeads dried at room temperature. Following drying, the minibeads are passed through a 1 to 10 mm, optionally 2 to 5 mm to remove oversized minibeads and then through a sieve with a pore size of 0.5 to 9 mm optionally 1 to 4 mm to remove undersized minibeads.

It can be appreciated that it is possible to recycle the minibeads that are rejected by the sieving process.

The bead-forming machines may be adapted to make use of a dual concentric lumen nozzle to ensure simultaneous extrusion of two fluids, the fluid in the inner lumen forming a core and the fluid of the outer lumen forming a capsule. The fluid forming the capsule is solidified according to one of the methods described. It may or may not be desirable for the fluid forming the core to be susceptible of solidification to yield a particular embodiment of the composition of the invention.

The above machinery adapted in this way can be used to manufacture the composition of the invention in the form of a capsule in which the core of the composition is filled with a fluid (a gas or a liquid) as described in the section above entitled “Shape, Size and Geometry” (noting that the core, like the capsular material, may be a composition, albeit optionally a distinct composition, according to the invention i.e. susceptible of solidification according to one of the methods described above). A three-lumen nozzle and appropriate tubing may be employed if it is desired to include an intermediate internal layer e.g. internal film layer of non-aqueous material on the inner face of the sphere with the intermediate layer conveniently being solid at room temperature. Thus, in terms of the softness/hardness of successive layers, the composition may for example be described as solid:solid in the case of two layers or solid:solid:solid in the case of 3 layers or liquid/semi-liquid:solid:solid in the case of 3 layers.

As a further aspect of the invention there is provided a composition obtainable by (having the characteristics of) any of the processes described herein.

The preceding paragraphs describe the formation of uncoated minibeads. It is a preferred embodiment of the present invention to have coated minibeads which are described in more detail elsewhere herein. Such coatings may be single or multiple and may be applied in a number of ways (see separate section).

With regard to one of the methods described above (ejection of emulsion through an optionally vibrating nozzle) with two concentric orifices (centre and outer), the outer fluid may form a coat (outside the minibead) of e.g. polymeric material (polymeric coating) which may contain an active principle or may impart controlled release characteristics to the minibead and the inner layer (core) may be a composition according to the invention. The Spherex machine manufactured by Freund (see U.S. Pat. No. 5,882,680 to Freund) may be used (the entire contents of this patent is incorporated herein by reference). Other similar ejection or extrusion apparatus may also be used, for example the ejection apparatus described hereinbefore.

Use of the Spherex machine achieves very high monodispersity. For example, in a typical 100 g, batch 97 g of minibeads were between 1.4 to 2 mm diameter or between 1 and 2 mm. Desired size ranges can be achieved by methods known in the art for rejecting/screening different sized particles. For example, it is possible to reject/screen out the larger/smaller minibeads by passing a batch first through e.g. a 2 mm mesh and subsequently sieved between 1 and 1.3 mm mesh.

The 1 to 2mm or 1 to 2.5 mm diameter range is a good size if it is desired to coat the minibeads (if smaller, the spray of the coating machine may bypass the minibead; if too large, the minibeads may be harder to fluidise which is necessary to achieve consistent coating).

The minibeads are preferably internally (i.e. cross-sectionally) homogeneous i.e. monolithic, though of course heterogeneous with surfactant and polymer phases on a micro-scale, although processing conditions may be varied for example by altering the temperature of the fluid emulsion, the solidification fluid and the concentration of components in these fluids and the time allowed for certain processing steps to occur including drying. Although not currently preferred, such variations may be applied in the case of minibead manufacture to achieve heterogeneity such as, for example, a harder skin and softer core with less than complete immobilization of non-ionic surfactant droplets towards the core as opposed to the surface of the minibead. Larger (e.g. non-beaded) forms or shapes of the composition according to the invention may particularly be engineered to embody such heterogeneity. However, it is currently preferred to have internally homogenous compositions according to the invention and within the minibead embodiment, this can be favoured by conducting the minibeading/dropletting using a homogeneous medium e.g. well dispersed micelles. Such homogeneity in the micelle dispersion to be minibeaded can help avoid the drying conditions affecting symmetry.

The invention further provides a product having the characteristics of a composition obtained as described herein, a product defined in terms of its characteristics being defined by the characteristics of the composition to the exclusion of the method by which it was made.

### Coating and Controlled Release

A coating may be applied to the minibeads for targeted, controlled, delayed and/or sustained release of the celecoxib. Application of the appropriate coat may, for example if colonic release is required, allow for say less than 10% of the active principle to be dissolved (in dissolution medium) at 4 hours and then a burst (sudden release) towards a maximum dissolution (approaching 100%) in the subsequent 24 hours. Many alternative target profiles are possible and this example is purely for illustration.

Thus according to one embodiment of the present invention, there is provided a dosage form comprising a population of minibeads as described herein, at least some of which and optionally all of which bear a coat (i.e. are coated) in order to control release of celecoxib from the minibead. In one embodiment, the coat is a film and, in another embodiment, it is a membrane. The coat, e.g. film or membrane, may serve to delay release until after the stomach; the coat may therefore be an enteric coat. The coat may be pH-dependent. The coat may comprise one or more substances preferably of a polymeric nature (e.g. methacrylates etc; polysaccharides etc as described in more detail below) or combination of more than one such substance, optionally including other excipients or active principles, such as, for example, plasticizers, described e.g. in the sections above on active principles. Preferred plasticizers, if they are used, include hydrophilic plasticizers for example triethyl citrate (TEC) which is particularly preferred when using the Eudragit® family of polymers as coatings as described below. Another preferred plasticiser, described in more detail below in relation to coating with ethyl cellulose, is DBS. Alternative or additional optionally included excipients are glidants. A glidant is a substance that is added to a powder or other medium to improve its flowability. A typical glidant is talc which is preferred when using the Eudragit® family of polymers as coatings.

In embodiments of the invention the composition comprises a hydrogel-forming polymer and further polymers able to achieve a desired delay (or other change) in the release of the drug and/or poration of the coating and/or exposure of the composition within the coating to allow egress of drug and/or dissolution of the immobilization matrix. In one embodiment, the composition comprises two types of polymers, which are combined into the same polymeric material, or provided as separate coats that are applied to the composition.

Controlled release can be achieved without an additional coating. In this case the polymer matrix comprises a further polymer aimed at a controlled release of an active ingredient. While mixtures of hydrogel-forming polymers are contemplated by the invention, the composition of the present invention in many embodiments comprises a polymer matrix material which is substantially a single material or type of material among those described herein and/or a matrix which can be solidified without inclusion of specific additional polymeric components in the aqueous phase. However, mixtures may be preferred to achieve certain performance characteristics. Thus it may be desired to incorporate certain constraining or retarding substances (retardants) into the water-soluble polymer matrix. In certain embodiments, such incorporation permits a coat (or coating) to be dispensed with. In other embodiments where a constraining or retarding agent is included into the water-soluble polymer matrix, a coat (or coating) may be present and desirable. For example, incorporation of a retarding agent which is insoluble in acid milieu (such as the stomach) is selected to prevent or retard release in the stomach and a coating may not be needed i.e. the composition may be free of a coat/coating. Alternatively, incorporation of a retarding agent which is soluble in acid media may be selected to retard release in the intestine distal to the stomach. Again a coating may not be needed i.e. the composition may be free of a coat/coating. However, the composition according to the invention which incorporates a retarding agent soluble in acid media may optionally be coated e.g. with an acid-resistant polymer to achieve particular advantage. Such a composition is protected from (complete) gastric release (or gastric release is retarded) owing to the effect of the acid-resistant polymer coat. Distal to the stomach, following loss of the coat, the acid-soluble agent retards release because the milieu of the small and large intestine is no longer acid.

Retarding or constraining agents insoluble in acid milieu include polymers whose solubility is pH-dependent i.e. soluble at higher pH. Such polymers are described in detail in the section below entitled “Coating” and such polymers may be used either as coats/coatings or as retarding agents incorporated into the water-soluble polymer matrix. An example of a suitable retarding agent mentioned in the section below entitled “Coating” is HPMCP (hydroxy-propyl-methyl-cellulose-phthalate also known as hypromellose phthalate) which is used to prevent release in the gastric environment since it is soluble above pH 5.5—see that section for other examples of polymers soluble in non-acid (basic) media. HPMCP may also be used as a pore-former. Retarding or constraining agents soluble in acid milieu include polymers whose solubility is pH-dependent i.e. soluble at lower pH. Such polymers include cationic polymers such as for example copolymers based on dimethylaminoethyl methacrylate, butyl methacrylate, and methyl methacrylate. An example of such a cationic co-polymer which may be used according to the invention is Eudragit E PO commercially available from Evonik Industries.

It has previously been stated that the dosage form of the invention may comprise more than one population of minibeads. Within the coating embodiment, the differences between populations may lie in the coat i.e. two (or more) populations of minibeads may differ in a number of respects one of which is the coating.

The coat may be applied as described below and may vary as to thickness and density. The amount of coat is defined by the additional weight added to (gained by) the dry composition (e.g. minibead) of the invention. Weight gain is preferably in the range 0.1% to 50%, preferably from 1% to 15% of the dry weight of the minibead, more preferably in the range 3% to 10% or in the range 5-12% or in the range 8-12%.

The polymeric coating material may comprise methacrylic acid co-polymers, ammonio methacrylate co-polymers, or mixtures thereof. Methacrylic acid co-polymers such as, for example, EUDRAGIT™ S and EUDRAGIT™ L (Evonik) are particularly suitable. These polymers are gastroresistant and enterosoluble polymers. Their polymer films are insoluble in pure water and diluted acids. They may dissolve at higher pHs, depending on their content of carboxylic acid. EUDRAGIT™ S and EUDRAGIT™ L can be used as single components in the polymer coating or in combination in any ratio. By using a combination of the polymers, the polymeric material can exhibit solubility at a variety of pH levels, e.g. between the pHs at which EUDRAGIT™ L and EUDRAGIT™ S are separately soluble. In particular, the coating may be an enteric coating comprising one or more co-polymers described in this paragraph. A particular coating material to be mentioned is Eudragit L 30 D-55.

The trademark “EUDRAGIT” is used hereinafter to refer to methacrylic acid copolymers, in particular those sold under the EUDRAGIT™ by Evonik.

The coating can comprise a polymeric material comprising a major proportion (e.g., greater than 50% of the total polymeric coating content) of at least one pharmaceutically acceptable water-soluble polymer, and optionally a minor proportion (e.g., less than 50% of the total polymeric content) of at least one pharmaceutically acceptable water insoluble polymer. Alternatively, the membrane coating can comprise a polymeric material comprising a major proportion (e.g., greater than 50% of the total polymeric content) of at least one pharmaceutically acceptable water insoluble polymer, and optionally a minor proportion (e.g., less than 50% of the total polymeric content) of at least one pharmaceutically acceptable water-soluble polymer.

Ammonio methacrylate co-polymers such as, for example, EUDRAGIT™ RS and EUDRAGIT™ RL (Evonik) are suitable for use in the present invention. These polymers are insoluble in pure water, dilute acids, buffer solutions, and/or digestive fluids over the entire physiological pH range. The polymers swell in water and digestive fluids independently of pH. In the swollen state, they are then permeable to water and dissolved active agents. The permeability of the polymers depends on the ratio of ethylacrylate (EA), methyl methacrylate (MMA), and trimethylammonioethyl methacrylate chloride (TAMCI) groups in the polymer. For example, those polymers having EA:MMA:TAMCI ratios of 1:2:0.2 (EUDRAGIT™ RL) are more permeable than those with ratios of 1:2:0.1 (EUDRAGIT™ RS). Polymers of EUDRAGIT™ RL are insoluble polymers of high permeability. Polymers of EUDRAGIT™ RS are insoluble films of low permeability. A diffusion-controlled pH-independent polymer in this family is RS 30 D which is a copolymer of ethyl acrylate, methyl methacrylate and a low content of methacrylic acid ester with quaternary ammonium groups present as salts to make the polymer permeable. RS 30 D is available as an aqueous dispersion.

The amino methacrylate co-polymers can be combined in any desired ratio, and the ratio can be modified to modify the rate of drug release. For example, a ratio of EUDRAGIT™ RS: EUDRAGIT™ RL of 90:10 can be used. Alternatively, the ratio of EUDRAGIT™ RS: EUDRAGIT™ RL can be about 100:0 to about 80:20, or about 100:0 to about 90:10, or any ratio in between. In such formulations, the less permeable polymer EUDRAGIT™ RS generally comprises the majority of the polymeric material with the more soluble RL, when it dissolves, permitting gaps to be formed through which solutes can come into contact with the minibead allowing pre-dissolved pharmaceutical actives to escape in a controlled manner.

The amino methacrylate co-polymers can be combined with the methacrylic acid co-polymers within the polymeric material in order to achieve the desired delay in the release of the drug and/or poration of the coating and/or exposure of the composition within the coating to allow egress of drug and/or dissolution of the immobilization or water-soluble polymer matrix. Ratios of ammonio methacrylate co-polymer (e.g., EUDRAGIT™ RS) to methacrylic acid co-polymer in the range of about 99:1 to about 20:80 can be used. The two types of polymers can also be combined into the same polymeric material, or provided as separate coats that are applied to the minibeads.

Eudragit™ FS 30 D is an anionic aqueous-based acrylic polymeric dispersion consisting of methacrylic acid, methyl acrylate, and methyl methacrylate and is pH sensitive. This polymer contains fewer carboxyl groups and thus dissolves at a higher pH (>6.5). The advantage of such a system is that it can be easily manufactured on a large scale in a reasonable processing time using conventional powder layering and fluidized bed coating techniques. A further example is EUDRAGIT® L 30D-55 which is an aqueous dispersion of anionic polymers with methacrylic acid as a functional group. It is available as a 30% aqueous dispersion.

In addition to the EUDRAGIT™ polymers described above, a number of other such copolymers can be used to control drug release. These include methacrylate ester co-polymers such as, for example, the EUDRAGIT™ NE and EUDRAGIT™ NM ranges. Further information on the EUDRAGIT™ polymers can be found in “Chemistry and Application Properties of Polymethacrylate Coating Systems,” in Aqueous Polymeric Coatings for Pharmaceutical Dosage Forms, ed. James McGinity, Marcel Dekker Inc., New York, pg 109-114 the entirety of which is incorporated herein by reference.

Several derivatives of hydroxypropyl methylcellulose (HPMC) also exhibit pH dependent solubility and may be used in the invention for coating. These include hydroxypropyl methylcellulose phthalate (HPMCP), which rapidly dissolves in the upper intestinal tract and hydroxypropyl methylcellulose acetate succinate (HPMCAS) in which the presence of ionisable carboxyl groups causes the polymer to solubilize at high pH (>5.5 for the LF grade and >6.8 for the HF grade). These polymers are commercially available from Shin-Etsu Chemical Co. Ltd. As with other polymers described herein as useful for coatings, HPMC and derivatives may be combined with other polymers e.g. EUDRAGIT RL-30 D.

There may be used a polymeric coating substance which is pH-independent in its dissolution profile and/or in its ability to release active principles incorporated in the compositions of the invention. Examples have already been given (e.g., Eudragit RS and RL). Another example of a pH-independent polymeric coating substance is ethylcellulose. It will be understood that an ethylcellulose composition for use in coating a dosage form for may comprise, in addition to ethylcellulose and—in the case of a liquid composition—a liquid vehicle, one or more other components. The other components may serve to modulate the properties of the composition, e.g. stability. The ethylcellulose may be the sole controlled release polymer in such a composition. The ethylcellulose may be in an amount of at least 50%, at least 60%, at least 70%, at least 80%, at least 90% or at least 95% by weight of the dry weight of composition for use in coating a dosage form. Accordingly, an ethylcellulose coating may include other components in addition to the ethylcellulose. The ethylcellulose may be in an amount of at least 50%, at least 60%, at least 70%, at least 80%, at least 90% or at least 95% by weight of the ethylcellulose coating.

A particular ethylcellulose coating composition which may be applied to the compositions of the invention is a dispersion of ethylcellulose in a sub-micron to micron particle size range, e.g. from about 0.1 to 10 microns in size, homogeneously suspended in water with the aid of an emulsification agent, e.g. ammonium oleate. The ethylcellulose dispersion may optionally and preferably contain a plasticizer, for example dibutyl sebacate (DBS) or medium chain triglycerides. Such ethylcellulose dispersions may, for example, be manufactured according to U.S. Pat. No. 4,502,888, which is incorporated herein by reference. One such ethylcellulose dispersion suitable for use in the present invention and available commercially is marketed under the trademark Surelease®, by Colorcon of West Point, Pa. USA. In this marketed product, the ethylcellulose particles are, e.g., blended with oleic acid and a plasticizer, then optionally extruded and melted. The molten plasticized ethylcellulose is then directly emulsified, for example in ammoniated water optionally in a high shear mixing device, e.g. under pressure. Ammonium oleate can be formed in situ, for instance to stabilize and form the dispersion of plasticized ethylcellulose particles. Additional purified water can then be added to achieve the final solids content. See also U.S. Pat. No. 4,123,403, which is incorporated herein by reference.

The term “Surelease®-type” is used hereinafter to refer to ethylcellulose coating materials, for example a dispersion of ethylcellulose in a sub-micron to micron particle size range, e.g. from about 0.1 to 10 microns in size, homogeneously suspended in water with the aid of an emulsification agent, e.g. ammonium oleate. In particular, a Surelease®-type material is the product marketed by Colorcon under the Surelease® trademark.

Surelease®-type dispersion is an example of a combination of film-forming polymer, plasticizer and stabilizers which may be used as a coating to adjust rates of active principle release with reproducible profiles that are relatively insensitive to pH. The principal means of drug release is by diffusion through the Surelease®-type dispersion membrane and is directly controlled by film thickness. It is possible to increase or decrease the quantity of Surelease®-type material, e.g. Surelease®, applied as coating in order to modify the dissolution of the coated composition. Unless otherwise stipulated, use of the term “Surelease” may apply to Surelease E-7-19020, E-7-19030, E-7-19040 or E-7-19050 and to products having the composition of those Surelease® grades. E-7-19020 comprises ethylcellulose blended with oleic acid and dibutyl sebacate, then extruded and melted. The molten plasticized ethylcellulose is then directly emulsified in ammoniated water in a high shear mixing device under pressure. Ammonium oleate is formed in situ to stabilize and form the dispersion of plasticized ethylcellulose particles. Additional purified water is then added to achieve the final solids content. E-7-19030 additionally comprises colloidal anhydrous silica dispersed into the material. E-7-19040 is like E-7-19020 except that it comprises medium chain triglycerides instead of dibutyl sebacate. E-7-19050 derives from blending ethylcellulose with oleic acid before melting and extrusion. The molten plasticized ethylcellulose is then directly emulsified in ammoniated water in a high shear mixing device under pressure. Ammonium oleate is formed in situ to stabilize and form the dispersion of plasticized ethylcellulose particles. However, E-7-19040 is preferred.

The invention also contemplates using combinations of Surelease®-type materials with other coating components, for example sodium alginate, e.g. sodium alginate available under the trade name Nutrateric™.

In addition to the EUDRAGIT™ and Surelease® polymers discussed above, other polymers may be used, in particular enteric, or pH-dependent, polymers. Such polymers can include phthalate, butyrate, succinate, and/or mellitate groups. Such polymers include, but are not limited to, cellulose acetate phthalate, cellulose acetate succinate, cellulose hydrogen phthalate, cellulose acetate trimellitate, hydroxypropyl-methylcellulose phthalate, hydroxypropylmethylcellulose acetate succinate, starch acetate phthalate, amylose acetate phthalate, polyvinyl acetate phthalate, and polyvinyl butyrate phthalate. Additionally, where compatible, any combination of polymer may be blended to provide additional controlled- or targeted-release profiles.

The coating can further comprise at least one soluble excipient to increase the permeability of the polymeric material. Suitably, the at least one soluble excipient is selected from among a soluble polymer, a surfactant, an alkali metal salt, an organic acid, a sugar, and a sugar alcohol. Such soluble excipients include, but are not limited to, polyvinyl pyrrolidone, polyethylene glycol, sodium chloride, surfactants such as, for example, sodium lauryl sulfate and polysorbates, organic acids such as, for example, acetic acid, adipic acid, citric acid, fumaric acid, glutaric acid, malic acid, succinic acid, and tartaric acid, sugars such as, for example, dextrose, fructose, glucose, lactose, and sucrose, sugar alcohols such as, for example, lactitol, maltitol, mannitol, sorbitol, and xylitol, xanthan gum, dextrins, and maltodextrins. In some embodiments, polyvinyl pyrrolidone, mannitol, and/or polyethylene glycol can be used as soluble excipients. The at least one soluble excipient can be used in an amount ranging from about 1% to about 10% by weight, based on the total dry weight of the polymer.

The modifications in the rates of release, such as to create a delay or extension in release, can be achieved in any number of ways. Mechanisms can be dependent or independent of local pH in the intestine, and can also rely on local enzymatic activity to achieve the desired effect. Examples of modified-release formulations are known in the art and are described, for example, in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566 all of which are incorporated herein by reference in their entirety.

The addition to Surelease™ or other pH-independent polymer substance, a second polymer (e.g. a polysaccharide, especially a heteropolysaccharide) which is susceptible to degradation by colonic bacterial enzymes (and optionally or alternatively by pancreatic or other relevant enzymes), provides targeted release of actives to a site or sites where the second polymer is degraded and flexibility in modulating the amount of polymer added to the composition of the invention in order to achieve optimal dissolution profiles.

The invention therefore also provides a coating for compositions of the invention intended to release their active payload in the colon which is a combination of ethylcellulose (preferably formulated with an emulsification agent such as, for example, ammonium oleate and/or a plasticizer such as, for example, dibutyl sebacate or medium chain triglycerides) and a polysaccharide susceptible to degradation by a bacterial enzyme normally found in the colon. Such polysaccharides include chondroitin sulphate, pectin, dextran, guar gum and amylase, chitosan etc and derivatives of any of the foregoing. Chitosan is particularly preferred in connection with obtaining a colon-specific release profile. The invention also includes a composition comprising a combination of ethylcellulose (preferably formulated with an emulsification agent such as, for example, ammonium oleate and/or a plasticizer such as, for example, dibutyl sebacate or medium chain triglycerides) and a polysaccharide susceptible to degradation by a bacterial enzyme normally found in the colon; the composition may include a liquid vehicle, e.g. water.

The use of polysaccharides by themselves for coating purposes has been tried with limited success. Most of the non-starch polysaccharides suffer from the drawback of lacking good film forming properties. Also, they tend to swell in the GI tract and become porous, resulting in the early release of the drug. Even amorphous amylose, which is resistant to degradation by pancreatic alpha amylase but capable of degradation by colonic bacterial enzymes has the disadvantage of swelling in aqueous media although this can be controlled by incorporating insoluble polymers like, for example, ethyl cellulose and acrylates into the amylose film. Amylose however is not water-soluble and, although water-insoluble polysaccharides are not excluded, use of a water-soluble polysaccharide (WSP) susceptible of bacterial enzymic degradation may bring particularly advantageous results when used as a coating. A particularly preferred polysaccharide in this embodiment of the present invention is pectin. Various kinds of pectin may be used including pectin of different grades available i.e. with differing degrees of methylation (DM), i.e. percentage of carbonyl groups esterified with methanol, for example pectins with a DM of more than 50%, known as High Methoxy (HM) Pectins or Low Methoxy (LM) pectins, or a pectin combination comprising an HM pectin and an LM pectin. It is also possible in this embodiment to use pectins having various degrees of acetylation (DAc). Taken together, the DM and DAc or the degree of substitution is known as Degree of Esterification (DE). Pectins of various DE's may be used according to the invention. As an alternative to pectin, sodium alginate may be used as a polysaccharide according to an embodiment of the invention. However, other embodiments may conveniently include amylose and/or starch which contains amylose. Various grades of starch, containing different percentages of amylose may be used including for example Hylon V (National Starch Food Innovation) which has an amylose percentage of 56% or Hylon VII which has an amylose percentage of 70%. The remaining percentage is amylopectin. The polysaccharides pectin, amylose and sodium alginate are particularly preferred for achieving colon delivery i.e. for compositions intended to release active principles in the colon.

It has been found that pectin can act as a former of pores in the coating otherwise provided by ethylcellulose (preferably Surelease). By “pores” is not meant shaft-like holes from the surface to the core of the composition, rather areas of weakness or absence of coating occurring stochastically on and within the coating of the invention.

A water-soluble polysaccharide may therefore be use as a pore-former in a coating, for example a pH-independent coating, e.g. a coating comprising ethylcellulose. According to a particular embodiment of the invention, where the water-soluble polysaccharide (WSP) is pectin, the proportion of Surelease®-type material, e.g. Surelease®, to pectin is ideally in the range 90:10 to 99:1, preferably, 95:5 to 99:1, more preferably 98:2 to 99:1.

In this particularly preferred combination (Surelease®-type material+WSP e.g. pectin) the weight gain and ratio between Surelease®-type material and WSP can be varied to refine the behaviour of the coating and the composition of the invention when it bears such a coat. Thus, the advantages of this preferred combination of coating polymers were further pronounced by selecting a weight gain in the range 0 to 30% (preferably 5 to 10%) and a Surelease®-type to pectin ratio in the range 95:5 to 99.5:0.5 preferably 97:3 to 99:1 inclusive. Particularly favoured weight gains using Surelease®-type material are those in the range 5-12% or in the range 8-12%.

Although the focus above has been on extending, delaying and/or sustaining release of active principles from compositions according to the invention, also contemplated are uncoated compositions, coated immediate release compositions for gastric release and simple gastro-protected, e.g. enteric coated, compositions providing early, small intestinal active ingredient release with sufficient coating, e.g. enteric coating, merely to protect the composition from dissolution in the stomach.

It is preferred to dry the compositions of the invention before they are coated with a suitable polymeric coat (as described in more detail above/below).

The coating process can be carried out by any suitable means such as, for example, by use of a coating machine which applies a solution of a polymer coat (as described above in particular) to the composition. Polymers for coating are either provided by the manufacturer in ready-made solutions for direct use or can be made up before use following manufacturers' instructions.

Appropriate coating machines are known to persons skilled in the art and include, for example, a perforated pan or fluidized-based system for example the GLATT, Vector (e.g. CF 360 EX), ACCELACOTA, Diosna, O'Hara and/or HICOATER processing equipment. To be mentioned is the MFL/01 Fluid Bed Coater (Vector/Freund) used in the “Bottom Spray” configuration.

Typical coating conditions are as follows:

| Process Parameter | Values |
|---|---|
| Fluidising airflow (m3/h) | 20-60 (preferably 30-60) |
| Inlet air temperature (° C.) | 20-65 |
| Exhaust air temperature (° C.) | 20-42 |
| Product temperature (° C.) | 20-42 |
| Atomizing air pressure (bar) | Up to 1.4 e.g. 0.8-1.2 |
| Spray rate (g/min) | 2-10 and 3-25 RPM |

The optionally coated minibeads of the invention may be formulated directly following their manufacture in the ways described above. In an alternative embodiment, it may be desired to impart different properties to the minibeads and/or to a final dosage form. One way of achieving this according to the invention is through granulation e.g. to improve the flow of powder mixtures of minibeads with other components as e.g. described above in relation to binders. Granules of intact or broken minibeads may be obtained by adding liquids (e.g. binder or solvent solutions) and effecting a granulating step as described in the prior art. Larger quantities of granulating liquid produce a narrower particle size range and coarser and harder granules, i.e. the proportion of fine granulate particles decreases. The optimal quantity of liquid needed to get a given particle size may be chosen in order to minimise batch-to-batch variations. According to this embodiment, wet granulation is used to improve flow, compressibility, bio-availability, homogeneity, electrostatic properties, and stability of the composition of the invention presented as a solid dosage form. The particle size of the granulate is determined by the quantity and feeding rate of granulating liquid. Wet granulation may be used to improve flow, compressibility, bio-availability, and homogeneity of low dose blends, electrostatic properties of powders, and stability of dosage forms. A wet granulation process according to this embodiment may employ low or high shear mixing devices in which a low viscosity liquid (preferably water) is added to a powder blend containing binder previously dry mixed with the rest of the formulation including minibeads. Alternative granulation approaches which may be utilized include high-shear, extrusion and conventional wet granulation.

### Dosage Forms

In a further aspect, the present invention provides for a dosage form comprising a population of minibeads of the invention. The minibeads of the dosage form may optionally be coated (as described above). The dosage form may comprise at least two populations of minibeads. The dosage form may comprise a single population of minibeads.

The dosage form is obtainable by preparing a minibead as described above. Optionally, the minibead is coated; the optional coating may be formulated in such a way as to provide a known or desired release profile in the gastrointestinal tract (GIT). A population of minibeads is then formulated into a suitable single unit dosage form (as described below) by procedures known to those skilled in the art to produce the dosage form. The dosage form may be further processed (e.g. by coating) to allow a modified release rate of the active ingredient in the GIT.

The dosage form comprises a population of minibeads of the invention in a unit dosage form suitable for administration, for example to a human or animal. The unit dosage form may be chosen from a capsule, a tablet, a sprinkle, a sachet, a suppository, a pessary or other suitable unit dosage form.

In embodiments the dosage form comprising a population of minibeads may be presented in a single unit dosage form e.g. contained in a single hard gel capsule which releases the minibeads e.g. in the stomach. Alternatively the minibeads may be presented in a sachet or other container which permits the minibeads to be sprinkled onto food or into a drink or to be administered via a feeding tube for example a naso-gastric tube or a duodenal feeding tube. Alternatively, the minibeads may be administered as a tablet for example if a population of minibeads is compressed into a single tablet as described below. Alternatively, the minibeads may be filled e.g. compressed into a specialist bottle cap or otherwise fill a space in a specialised bottle cap or other element of a sealed container (or container to be sealed) such that e.g. on twisting the bottle cap, the minibeads are released into a fluid or other contents of the bottle or vial such that the minibeads are dispersed (or dissolve) with or without agitation in such contents. An example is the Smart Delivery Cap manufactured by Humana Pharma International (HPI) S.p.A, Milan, Italy.

The dosage form may be formulated in such a way so that the minibeads of the invention can be further developed to create a larger mass of minibeads e.g. via compression (with appropriate oil or powder-based binder and/or filler known to persons skilled in the art of pharmaceutical formulation and with the option of including additional quantities of the same API as in the composition of the invention or a different API a preferred example being where the composition of the invention takes the form of minibeads which comprise immediate or controlled release celecoxib of a plurality of minibeads which disintegrate at a different rate in different conditions than a unitary moulded form of the same shape. The larger (e.g. compressed) mass may itself take a variety of shapes including pill shapes, tablet shapes, capsule shapes etc. A particular problem which this version of the minibead embodiment solves is the “dead space” (above the settled particulate contents) and/or “void space” (between the particulate content elements) typically found in hard gel capsules filled with powders or pellets. In such pellet- or powder-filled capsules with dead/void space, a patient is required to swallow a larger capsule than would be necessary if the capsules contained no such dead space. The minibeads of this embodiment of the invention may readily be compressed into a capsule to adopt the inner form of whichever capsule or shell may be desired leaving much reduced, e.g. essentially no, dead/void space. Alternatively the dead or void space can be used to advantage by suspending minibeads in a vehicle such as, for example, an oil which may be inert or may have functional properties such as, for example, permeability enhancement or enhanced dissolution or may comprise an active ingredient being the same or different from any active ingredients in the minibead. For example, hard gelatin capsules may be filled with a liquid medium combined with uncoated and/or coated minibeads. The liquid medium may be, or comprise, a non-ionic surfactant as described herein; it may be, or comprise, one or more oils. Particularly preferred but non-limiting examples are corn oil, sorbitane trioleate (sold under the trade mark SPAN 85), propylene glycol dicaprylocaprate (sold under the trade mark Labrafac), 2-(2-ethoxyethoxy)ethanol (sold under the trade mark Trancutol P) and polysorbate 80 (sold under the trade mark Tween 80).

The minibeads so-presented may be of a single type (or population) or may be of multiple types (or populations) differing between populations in relation to one or more features described herein e.g. different active ingredient/active ingredient combination or different excipients or different physical geometry, coated, multiply coated, uncoated etc.

In some embodiments the dosage form has been appropriately formulated in such a way as to release the celecoxib and any other active ingredients at predetermined region(s) the GIT e.g. the colon. The appropriate formulation may be achieved by coating the minibeads.

As mentioned elsewhere herein, the pharmaceutical formulations of the invention may be for oral or rectal administration. The dosage form of the invention, therefore, may be suitable for oral administration.

The invention includes oral dosage forms comprising multiple, e.g. at least 10, shaped units, e.g. minibeads, of the invention, therefore.

In particular, the formulations may be multiple minibead formulations, for example comprising at least 10 minibeads. Exemplarily, the multiple minibeads are contained in a capsule.

A multiple minibead formulation may contain a single population of minibeads. The members of the population may therefore substantially be the same within the tolerance of the manuracturing process.

In some implementations of the invention, the members of a minibead population have a release profile to release celecoxib at least in the colon.

A multiple minibead formulation may comprise at least two populations of minibeads, where the two populations may differ as to the identity of the drug(s) they contain and/or as to release profile, provided always that one of the populations comprises minibeads of the invention. In a particular multiple minibead formulation, the minibeads are all minibeads of the invention and the formulation comprises:
1) a first population of minibeads that are adapted to release celecoxib in a first region of the gastrointestinal tract; and
2) a second population of minibeads that are adapted to release celecoxib in a second, e.g. lower, region of the gastrointestinal tract.

The first population may be adapted to release celecoxib in the stomach or in the small intestine, or in both. The first population may therefore be uncoated or have an immediate release coating for gastric release, or an enteric or other coating adapted to release the celecoxib in the small intestine. The first population may sustain release. The first population may be adapted to release the celecoxib in the duodenum and/or jejenum.

The second population may be delayed release minicapsules. The second population may both delay and sustain release. The second population may be adapted to release celecoxib in the ileum and/or the colon. The second population may be adapted to release celecoxib in at least the colon.

The first population may be adapted to release celecoxib in the stomach and the second population may be adapted to release celecoxib in the colon and/or ileum. The first population may be adapted to release celecoxib in the upper small intestine (duodenum and/or ileum) and the second population may be adapted to release celecoxib in the colon and/or ileum. The first population may be adapted to release celecoxib in the stomach and the second population may be adapted to release celecoxib in at least the upper small intestine.

To be mentioned also is a multiple minibead formulation that comprises:
1) a first population of minibeads that are minibeads of the invention; and
2) a second population of minibeads that are not of the invention, e.g. contain a different drug, optionally selected from (i) drugs for the prophylaxis and/or therapy of a gastrointestinal cancer, (ii) drugs for the prophylaxis and/or therapy of inflammation and/or an inflammatory condition of the gastrointestinal tract, and (iii) immunosuppressants.

If desired, third and optionally further minibead populations having different release profiles and/or drugs from the other populations may be incorporated in a multiple minibead formulation.

As is well known, controlled release technology may be used to control the release profile of the minibeads, for example the incorporation of controlled release polymers in the polymer matrix (in admixture with the hydrogel-forming polymer) and/or controlled release coatings may be applied to the minicapsules. Such controlled release technology may be used irrespective of the number of minibead populations in a multiple minibead formulation. Although controlled release technology is well established and will require no elaboration for the skilled reader, further information in relation to some aspects of controlled release technology, especially coatings, is contained elsewhere in this specification.

Where compositions include at least celecoxib and another active agent, the celecoxib and the other agent may be formulated for co-release or for sequential, e.g. pulsed, release.

### ADDITIONAL DISCLOSURE

The invention further includes the subject matters of the following paragraphs:
1. A composition that is a clear liquid comprising water, a hydrogel-forming polymer, celecoxib, a polyoxyethylated non-ionic surfactant and a precipitation inhibitor, wherein the celecoxib is in an amount of at least 2% by weight, calculated on the dry weight of the total composition.
2. The composition of paragraph 1 that comprises at most 5% by weight, calculated on the dry weight of the composition, of 2-(2-ethoxyethoxy)ethanol.
3. The composition of paragraph 2 that is free of 2-(2-ethoxyethoxy)ethanol.
4. The composition of any preceding paragraph that comprises at most 5% by weight, calculated on the dry weight of the composition, of hydrophobic molecules other than celecoxib.
5. The composition of paragraph 4 that comprises at most 1% by weight, calculated on the dry weight of the composition, of hydrophobic molecules other than celecoxib.
6. The composition of any preceding paragraph that is free of hydrophobic molecules other than celecoxib.
7. The composition of any preceding paragraph that comprises at most 5% by weight, calculated on the dry weight of the composition, of triglycerides.
8. The composition of paragraph 7 that is free of triglycerides.
9. The composition of any preceding paragraph that comprises at most 5% by weight, calculated on the dry weight of the composition, of glycerides.
10. The composition of paragraph 9 that is free of glycerides.
11. The composition of any preceding paragraph that comprises at most 5% by weight, calculated on the dry weight of the composition, of oils.
12. The composition of paragraph 11 that is free of oils.
13. A composition that is a clear liquid comprising water, a hydrogel-forming polymer, celecoxib, a polyoxyethylated non-ionic surfactant and a precipitation inhibitor, wherein the celecoxib is in an amount of at least 2% by weight, calculated on the dry weight of the composition, of the total composition, the composition containing at most 5% by weight, calculated on the dry weight of the composition, of 2-(2-ethoxyethoxy)ethanol and at most 5% by weight, calculated on the dry weight of the composition, of hydrophobic molecules other than celecoxib.
14. A composition that is a clear liquid comprising water, a hydrogel-forming polymer, celecoxib, a polyoxyethylated non-ionic surfactant and a precipitation inhibitor, wherein the celecoxib is in an amount of at least 2% by weight, calculated on the dry weight of the composition, of the total composition, the composition being free of triglycerides.
15. The composition of paragraph 14 that comprises at most 5% by weight, calculated on the dry weight of the composition, of 2-(2-ethoxyethoxy)ethanol.
16. The composition of paragraph 15 that is free of 2-(2-ethoxyethoxy)ethanol.
17. The composition of any preceding paragraph wherein the polyoxyethylated non-ionic surfactant is in an amount of at least 40% and optionally of at least 45%, e.g. at least 50%, wherein the percentages are by weight based on the dry weight of the composition.
18. The composition of any preceding paragraph wherein the polyoxyethylated non-ionic surfactant is in an amount from 40% to 70% and optionally from 50% to 70%, e.g. 50% to 60% or 52.5% to 57.5%, wherein the percentages are by weight based on the dry weight of the composition.
19. The composition of any preceding paragraph wherein the weight ratio of the hydrogel-forming polymer to the non-ionic surfactant is from 1:1 to 1:3 and optionally from 1:1.3 to 1:2.6 or from 1:1.4 to 1:2.2.
20. The composition of paragraph 19 wherein the weight ratio of the hydrogel-forming polymer to the polyoxyethylated non-ionic surfactant is from 1:1.5 to 1:2.1, e.g. from 1:1.5 to 1:1.9.
21. The composition of any preceding paragraph wherein the precipitation inhibitor is in an amount of from 0.5% to 10% and optionally from 1% to 10%, e.g. from 1% to 5% , optionally from 3% to 5%, wherein the percentages are by weight based on the dry weight of the composition.
22. The composition of any preceding paragraph wherein the hydrogel-forming polymer is selected from thermotropic hydrogel-forming polymers and combinations thereof.
23. The composition of paragraph 22 wherein the hydrogel-forming polymer is selected from the group consisting of gelatin, agar, agarose, pectin, carrageenan, and chitosan, and combinations thereof.
24. The composition of paragraph 22 or paragraph 23 wherein the hydrogel-forming polymer comprises, or is, gelatin.
25. The composition of any preceding paragraph wherein the composition comprises the hydrogel-forming polymer in an amount of at least 25%, suitably at least 30%, for example from 25% to 40%, or optionally from 25% to 35% or 30% to 40%, e.g. 30% to 35%, wherein the percentages are by weight based on the dry weight of the composition.
26. The composition of any preceding paragraph that further comprises a plasticiser, for example a polyol, e.g. sorbitol or glycerine, or a combination thereof.
27. The composition of paragraph 26 wherein the plasticiser is present in the composition at up to 10% by dry weight of the composition, for example between 3 and 8%, or suitably between 4% and 6%.
28. The composition of any preceding paragraph wherein the polyoxyethylated non-ionic surfactant comprises, or is, a combination of a polyoxyethylated aliphatic acid, e.g. a polyoxyethylated hydroxy aliphatic acid, and free polyethylene glycol.
29. The composition of paragraph 28 wherein the polyoxyethylated aliphatic acid comprises, or is, a combination of mono- and di-poly(oxyethylene) esters of a hydroxy fatty acid.
30. The composition of paragraph 29 wherein the polyoxyethylated non-ionic surfactant is macrogol-15-hydroxystearate (also known as polyoxyl-15-hydroxystearate).
31. The composition of any preceding paragraph wherein the precipitation inhibitor comprises, or is, an anionic surfactant or a cellulose polymer, or a combination thereof.
32. The composition of paragraph 31 wherein the precipitation inhibitor comprises, or is, a cellulose polymer or a combination thereof, e.g. hydroxypropyl cellulose and/or hydroxypropylmethyl cellulose.
33. The composition of paragraph 32 wherein the precipitation inhibitor comprises, or is, a mixture of hydroxypropyl cellulose, hydroxypropylmethyl cellulose, talc and TiO₂.
34. The composition of paragraph 31 wherein the precipitation inhibitor is an anionic surfactant.
35. The composition of paragraph 31 or paragraph 34 wherein the anionic surfactant comprises, or is, an alkyl sulfate salt, or a combination thereof.
36. The composition of paragraph 35 wherein the salt is a sodium salt.
37. The composition of paragraph 36 wherein the anionic surfactant is sodium dodecyl sulfate (SDS).
38. The composition of any preceding paragraph wherein the weight ratio of the precipitation inhibitor, e.g. anionic surfactant, to celecoxib is from 1:0.2 to 1:10 and optionally from 1:0.8 to 1:6 1:0.8 to 1:3, e.g. from 1:1 to 1:2.3.
39. The composition of any of paragraphs 34 to 37 wherein the weight ratio of the anionic surfactant to celecoxib is from 1:1 to 1:2, optionally from 1:1.2 to 1:1.6.
40. The composition of paragraph 32 or paragraph 33 wherein the weight ratio of the precipitation inhibitor to celecoxib is from 1:2 to 1:8, for example from 1:3 to 1:8 or from 1:3 to 1:7, particularly 1:3.5 to 1:5.5.
41. The composition of any preceding paragraph wherein the weight ratio of the polyoxyethylated non-ionic surfactant to celecoxib is from 35:1 to 4: 1, optionally from 13:1 to 5:1.
42. The composition of paragraph 41 wherein the weight ratio of the polyoxyethylated non-ionic surfactant to celecoxib is from 7.5:1 to 11.5:1, optionally about 9:1.
43. The composition of any preceding paragraph wherein the celecoxib is in an amount of 2% to 10% by weight based on the dry weight of the composition, e.g. 4 to 9%, 5 to 9% or 5 to 7%.
44. The composition of paragraph 26 or of any of the following paragraphs when including the feature of paragraph 26: paragraphs 1 to 16, 22 to 24 and 28 to 37, wherein the composition excluding any coatings comprises, or consists essentially of, the constituents in the amounts stated in the following table for any of the formulations designated A to F:

| | A | B | C | D | E | F |
|---|---|---|---|---|---|---|
| Constituent | Weight % | Weight % | Weight % | Weight % | Weight % | Weight % |
| Celecoxib | 2-10 | 2-10 | 4-10, e.g. | 4-9 | 5-9 | 5-7 |
| | | | 5-10 | | | |
| Non-ionic | 40-70 | 45-70, e.g. | 50-65 | 50-60 | 52.5-57.5 | 52.5-57.5 |
| surfactant | | 45-67 | | | | |
| Polymer | 25-40 | 25-40, e.g. | 25-37.5 | 25-35, e.g. | 28-35 | 30-35 |
| | | 28-40 | | 28-35 | | |
| Anionic | 0.5-10, | 1-5 | 1-5 | 3-5 | 3-5 | 3-5 |
| surfactant or | e.g. 1-10 | | | | | |
| Cellulose | 0.5-10, | 0.5-3 | 0.5-3 | 0.5-3 | 0.5-2 | 0.5-2 |
| polymer | e.g. 0.5-5 | | | | | |
| product | | | | | | |
| Plasticiser | 0-8, e.g. | 2-8 | 2-8 | 2-6 | 2-6, e.g. | 2-6, e.g. |
| | 2-8 | | | | 3-4 | 3-4 |

wherein: the anionic surfactant and the cellulose polymer are alternative precipitation inhibitors; the above percentages are by dry weight; in Formula B the amount of the hydrogel-forming polymer is from 28-40 when the amount of the non-ionic surfactant is from 45-67; and the total percentage contents of the constituents excluding water add up to 100.
45. The composition of any preceding paragraph wherein the identities of the constituents of the , are as set out in one of Columns (a) to (f) of the following table, for example the composition of paragraph 44 wherein the identities of the tabulated constituents of Formulations A to F are as set out in one of Columns (a) to (f) of the following table or as set out in a variant of Column (f) in which the precipitation inhibitor is as defined in Column (e):

| | (a) | (b) | (c) | (d) | (e) | (f) |
|---|---|---|---|---|---|---|
| Constituent | Identity | Identity | Identity | Identity | Identity | Identity |
| Non-ionic | Comprises | Comprises | Polyoxyl fatty | Polyoxyl | Polyoxyl- | Polyoxyl- |
| surfactant | a polyoxy- | a polyoxy- | acid | hydroxy fatty | 15- | 15- |
| | ethyl ester | ethylated | (macrogol | acid | hydroxy- | hydroxy- |
| | or ether | aliphatic | ester of fatty | | stearate | stearate |
| | | acid | acid) | | | |
| Polymer | Thermo- | Thermo- | At least one | Gelatin; and | Gelatin | Gelatin |
| | reversible | reversible | of gelatin; | optionally a | | |
| | hydrogel- | hydrogel- | agar; | minor | | |
| | forming | forming | agarose; | proportion of | | |
| | polymer(s) | polymer(s), | pectin; | agar; | | |
| | | gelatin at | carrageenan; | agarose; | | |
| | | least | chitosan. | pectin; | | |
| | | predominating | | carrageenan; | | |
| | | | | and/or | | |
| | | | | chitosan. | | |
| Precipitation | An anionic | An anionic | An aliphatic | An alkyl | A dodecyl | Sodium |
| Inhibitor | surfactant; | surfactant; | sulfate or | sulfate; or a | sulfate; or | dodecyl |
| | or a product | or a product | sulfonate; or | product that | HPC | sulfate; or |
| | that | that | a product that | comprises | and/or | HPC and |
| | comprises a | comprises | comprises | HPC and/or | HPMC, | HPMC |
| | cellulose | HPC and/or | HPC and/or | HPMC | optionally | optionally |
| | polymer | HPMC | HPMC | | together | together |
| | | | | | with (i) | with (i) |
| | | | | | TiO₂ | TiO₂ |
| | | | | | and/or (ii) | and/or (ii) |
| | | | | | talc, e.g. | talc, e.g. |
| | | | | | with both | with both |
| | | | | | (i) and (ii) | (i) and (ii) |
| Plasticiser | At least one | At least one | At least one | At least one | Sorbitol | Sorbitol |
| | polyol. | of glycerol, | of glycerol, | of glycerol, | | |
| | | sorbitol, | sorbitol, | sorbitol, | | |
| | | sorbitol/ | sorbitol/ | sorbitol/ | | |
| | | sorbitan | sorbitan | sorbitan | | |
| | | mixture, e.g. | mixture, e.g. | mixture, e.g. | | |
| | | sorbitol. | sorbitol. | sorbitol. | | |

and wherein the precipitation inhibitor in particular is SDS.
46. The composition of any preceding paragraph wherein the precipitation inhibitor is at least predominantly in the water.
47. A process of making minibeads, comprising ejecting the composition of any preceding paragraph through a nozzle to form droplets, the hydrogel-forming polymer then being caused or allowed to solidify whereby the droplets form minibeads, optionally wherein the minibeads are then dried.
48. The process of paragraph 47 that further comprises coating the minibeads.
49. The process of paragraph 48 wherein the coating is adapted for the minibeads to release the celecoxib at least in the colon.
50. The process of any of paragraphs 47 to 49 wherein the minibeads have a size of from 0.5 mm to 5 mm.
51. The process of any of paragraphs 47 to 50 that further comprises making the composition of any of paragraphs 1 to 46, the making of the composition comprising mixing:

i) a celecoxib solution comprising celecoxib and a polyoxyethylated non-ionic surfactant; and

ii) an aqueous solution comprising water, a hydrogel-forming polymer and a precipitation inhibitor;

wherein the two solutions are mixed to form a clear liquid.
52. A minibead having the characteristics of a minibead obtained by the process of any of paragraphs 47 to 51.
53. A composition comprising
   celecoxib,
   polyoxyethylated non-ionic surfactant in an amount of at least 40% by weight, calculated on the dry weight of the composition,
   a precipitation inhibitor, and
   a hydrogel-forming polymer matrix in which the celecoxib and the surfactants are included.
54. A composition comprising
   celecoxib,
   a polyoxyethylated non-ionic surfactant,
   a precipitation inhibitor, and
   a hydrogel-forming polymer in which the celecoxib and the surfactants are included;
      and wherein the composition has a feature selected from:
   (i) when combined with water, the composition is capable of releasing self-assembly structures comprising non-ionic surfactant and celecoxib;
   (ii) the polymer forms a matrix in which the polyoxyethylated non-ionic surfactant is distributed as inclusions that comprise celecoxib and have a size of less than 25μm.
55. The composition of paragraph 53 or paragraph 54 that comprises at most 5% by weight, calculated on the dry weight of the composition, of 2-(2-ethoxyethoxy)ethanol.
56. The composition of paragraph 55 that is free of 2-(2-ethoxyethoxy)ethanol.
57. The composition of any of paragraphs 53 to 56 that comprises at most 5% by weight, calculated on the dry weight of the composition, of hydrophobic molecules other than celecoxib.
58. The composition of paragraph 57 that comprises at most 1% by weight, calculated on the dry weight of the composition, of a hydrophobic molecules other than celecoxib.
59. The composition of any of paragraphs 53 to 58 that is free of hydrophobic molecules other than celecoxib.
60. The composition of any of paragraphs 53 to 59, the composition containing at most 5% by weight, calculated on the dry weight of the composition, of 2-(2-ethoxyethoxy)ethanol and at most 5% by weight, calculated on the dry weight of the composition, of hydrophobic molecules other than celecoxib.
61. The composition of any of paragraphs 53 to 60 that comprises at most 5% by weight, calculated on the dry weight of the composition, of triglycerides.
62. The composition of paragraph 61 that is free of triglycerides.
63. The composition of any of paragraphs 53 to 62 that comprises at most 5% by weight, calculated on the dry weight of the composition, of glycerides.
64. The composition of paragraph 63 that is free of glycerides.
65. The composition of any paragraphs 53 to 64 that comprises at most 5% by weight, calculated on the dry weight of the composition, of oils.
66. The composition of paragraph 65 that is free of oils.
67. The composition of any of paragraphs 53 to 66 wherein the celecoxib is in an amount of 2% to 10% by weight based on the dry weight of the composition.
68. The composition of paragraph 67 wherein the celecoxib is in an amount of 5% to 9% by weight based on the dry weight of the composition.
69. The composition of any of paragraphs 53 to 68 that further includes the specific feature(s) recited in any of paragraphs 17 to 45 or in a combination thereof permitted by dependency.
70. The composition of any of paragraphs 53 to 69 wherein the precipitation inhibitor at least predominantly shares the same space as the hydrogel-forming polymer.
71. The composition of any of paragraphs 53 to 70 that is suitable for oral administration.
72. The composition of paragraph 71 that further comprises at least one coating.
73. The composition of paragraph 72 wherein the at least one coating is adapted to release the celecoxib in at least the colon.
74. The composition of paragraph 73 that is adapted to release the celecoxib in the ileum and colon.
75. The composition of paragraph 73 that is adapted to release substantially all the celecoxib in the colon.
76. The composition of any of paragraphs 73 to 75 wherein the at least one coating comprises a pH-independent coating.
77. The composition of paragraph 76 wherein the pH-independent coating comprises ethylcellulose.
78. The composition of paragraph 76 or paragraph 77 wherein the pH-independent coating comprises a polymer susceptible to degradation by enzyme(s) of colonic bacteria.
79. The composition of any of paragraphs 72 to 75 wherein the at least one coating comprises a pH-dependent coating.
80. The composition of any of paragraphs 53 to 79 that is in the form of a minibead having a size of from 0.5 mm to 5 mm.
81. The composition of paragraph 80 wherein the minibead has a size of from 1 mm to 3 mm.
82. A multiple minibead formulation, comprising a unit dosage form comprising a multiplicity of minibeads of any of paragraphs 52, 80 and 81.
83. The formulation of paragraph 82 that comprises at least 10 of the minibeads.
84. The formulation of paragraph 82 or paragraph 83 wherein the unit dosage form is a capsule comprising the multiplicity of minibeads.
85. An oral celecoxib formulation, the formulation being a multiple minibead formulation wherein the minibeads comprise a hydrogel-forming polymer matrix in which are distributed celecoxib, a polyoxyethylated non-ionic surfactant and a precipitation inhibitor, wherein the non-ionic surfactant is in an amount of at least 40% by weight of the dry weight of the hydrogel-forming polymer matrix and substances in the matrix.
86. An oral celecoxib formulation, the formulation being a multiple minibead formulation wherein the minibeads comprise a hydrogel-forming polymer matrix in which are distributed celecoxib, a polyoxyethylated non-ionic surfactant and a precipitation inhibitor, the minibeads having a feature selected from:
   (i) the minibeads when combined with water are capable of releasing self-assembly structures comprising surfactant and celecoxib;
   (ii) the polymer forms a matrix in which the polyoxyethylated non-ionic surfactant is distributed as inclusions that comprise celecoxib and have a size of less than 25μm.
87. The formulation of paragraph 85 wherein the precipitation inhibitor at least predominantly shares the same space as the hydrogel-forming polymer or of paragraph 86 wherein the precipitation inhibitor is at least predominantly outside the inclusions.
88. The formulation of any of paragraphs 85 to 87 that further includes the specific feature(s) recited in any of paragraphs 53 to 84 or in a combination thereof permitted by dependency.
89. A product for use in treating colorectal cancer, wherein the product is selected from the minibead of paragraph 52, the composition of any of paragraphs 53 to 81 and the formulation of any of paragraphs 82 to 88, in any such case being for use in treating colorectal cancer.
90. The product of paragraph 89 that is for use in inhibiting, reducing or delaying the initiation and/or progression of colorectal cancer, or for use in causing regression of colorectal cancer, reducing the burden of colorectal cancer and/or inducing remission of colorectal cancer.
91. The product of paragraph 89 that is for use in inhibiting, reducing or delaying carcinogenesis of colorectal cancer.
92. The product of paragraph 89 that is for use in for use in causing regression of colorectal cancer, reducing the burden of colorectal cancer and/or inducing remission of colorectal cancer.
93. The product of paragraph 89 that is for use in inhibiting, reducing or delaying the progression of colorectal cancer.
94. The product of paragraph 89 that is for use in inhibiting, reducing or delaying metastasis of a colorectal cancer.
95. The product of any of paragraphs 89 to 94 wherein the colorectal cancer is a COX-2 positive adenocarcinoma.
96. The product of any of paragraphs 89 to 95 that is for use in administering celecoxib at a daily dosage of less than 800 mg/day.
97. The product of paragraph 96 that is for use in administering celecoxib at a daily dosage of no more than 500 mg/day.
98. The product of paragraph 96 that is for use in administering celecoxib at a daily dosage of less than 200 mg/day.
99. The product of paragraph 96 that is for use in administering celecoxib at a daily dosage of no more than 100 mg/day.
100. A product for use in treating colorectal inflammation or another gastrointestinal inflammatory condition wherein the product is selected from the minibead of paragraph 52, the composition of any of paragraphs 53 to 81 and the formulation of any of paragraphs 82 to 88, in any such case being for use in treating colorectal inflammation or another gastrointestinal inflammatory condition.
101. The product of paragraph 100 that is for use in inhibiting, reducing or delaying the initiation and/or progression of the inflammatory condition, or is for use in causing regression of or reducing the inflammatory condition.
102. The product of paragraph 100 or paragraph 101 that is for use in administering celecoxib at a daily dosage of less than 200 mg/day.
103. The product of paragraph 102 that is for use in administering celecoxib at a daily dosage of no more than 100 mg/day.
104. The product of any of paragraphs 91 to 103 that is for administering to patients who suffer from intolerance to another NSAID or having a history of hypersensitivity to another NSAID.
105. The product of any of paragraphs 91 to 104 wherein the celecoxib is for use in concurrent administration with another drug selected from anti-inflammatory drugs, immunosuppressants and drugs for the therapy or prophylaxis of cancer.
106. The product of paragraph 105 wherein said other drug is comprised in the product.
107. The product of paragraph 105 wherein said other drug is not in the product.
108. A process which comprises mixing a polyoxyethylated non-ionic surfactant and celecoxib to form a solution, wherein the weight ratio of the polyoxyethylated non-ionic surfactant to celecoxib is from 7.5:1 to 11.5:1, optionally about 9:1.
109. A process of paragraph 108 that further comprises mixing the solution with an aqueous solution comprising water, a hydrogel-forming polymer and a precipitation inhibitor to form a clear liquid.
110. A product (e.g. a composition of matter, a solution) that comprises, or is, a mixture of celecoxib and a polyoxyethylated non-ionic surfactant, wherein the weight ratio of the polyoxyethylated non-ionic surfactant to celecoxib is from 7.5:1 to 11.5:1, optionally about 9:1.
111. The subject matter of any of paragraphs 108 to 111 wherein the surfactant is as defined in any of paragraphs 28 to 30.
112. A solid composition comprising celecoxib contained in a polymer matrix that contains also a precipitation inhibitor, the polymer being a hydrogel-forming polymer, wherein the celecoxib is solubilised in a polyoxyethylated non-ionic surfactant and is in an amount of at least 2% by weight of the total composition, based on the dry weight of the composition consisting of the matrix and the substances included in the matrix.
113. A process of making a celecoxib composition, comprising mixing:

i) a celecoxib solution comprising celecoxib and a polyoxyethylated non-ionic surfactant; and

ii) an aqueous solution comprising water, a hydrogel-forming polymer and an precipitation inhibitor;

wherein the two solutions are mixed to form a clear liquid.
114. The process of paragraph 113 wherein the clear liquid is as further defined by the specific feature(s) of any of paragraphs 2 to 21, 25 and 39 to 45 or of any combination thereof permitted by dependency.
115. The process of paragraph 113 or paragraph 114 wherein the hydrogel-forming polymer is as defined by the specific feature(s) of any of paragraphs 22 to 24 or of any combination thereof permitted by dependency.
116. The process of any of paragraphs 113 to 115 wherein the aqueous solution further comprises a plasticiser, for example a polyol, e.g. sorbitol or glycerine, optionally wherein the plasticiser is present in the clear liquid in an amount as specified in paragraph 27.
117. The process of any of paragraphs 113 to116 wherein the polyoxyethylated non-ionic surfactant is as defined in any of paragraphs 28 to 30.
118. The process of any of paragraphs 113 to 117 wherein the precipitation inhibitor is as defined in any of paragraphs 31 to 37.
119. The process of any of paragraphs 113 to 118 wherein the celecoxib is in an amount of 2% to 10% by weight based on the dry weight of the composition.
120. The process of any of paragraphs 113 to 119 that further comprises ejecting the clear liquid through a nozzle to form droplets, the hydrogel-forming polymer then being caused or allowed to solidify whereby the droplets form minibeads, optionally wherein the minibeads are then dried.
121. The process of paragraph 120 that further comprises coating the minibeads, optionally to form at least one coating as defined in any of paragraphs 73 to 79.
122. A minibead having the characteristics of a minibead made by the process of paragraph 120 or paragraph 121.
123. A composition comprising
   celecoxib,
   a polyoxyethylated non-ionic surfactant,
   a precipitation inhibitor, and
   a hydrogel-forming polymer matrix in which the celecoxib , the surfactant and the precipitation inhibitor are included;
      wherein the non-ionic surfactant is present as inclusions within the polymer matrix and the precipitation inhibitor is at least predominantly outside the inclusions, and optionally wherein the composition when combined with water is capable of releasing self-assembly structures comprising non-ionic surfactant and celecoxib.
124. A composition comprising
   celecoxib,
   a polyoxyethylated non-ionic surfactant,
   a precipitation inhibitor, and
   a hydrogel-forming polymer in which the celecoxib, the surfactant and the precipitation inhibitorare included;
      the polymer forming a matrix in which the polyoxyethylated non-ionic surfactant is distributed as inclusions that comprise celecoxib and have a size of less than 25 μm, the precipitation inhibitor being at least predominantly outside the inclusions.
125. The composition of paragraph 123 or paragraph 124 that further includes the specific feature(s) recited in any of paragraphs 55 to 79 or in a combination thereof permitted by dependency.
126. The composition of any of paragraphs 123 to 125 that is in the form of a minibead having a size of from 0.5 mm to 5 mm, optionally a size of from 1 mm to 3 mm.
127. A multiple minibead formulation, comprising a unit dosage form comprising a multiplicity of minibeads of paragraph 122 or 126, optionally wherein the formulation is as defined in paragraph 83 and/or paragraph 84.
128. An oral celecoxib formulation, the formulation being a multiple minibead formulation wherein the minibeads comprise a hydrogel-forming polymer matrix in which are distributed celecoxib, a polyoxyethylated non-ionic surfactant and a precipitation inhibitor, the minibeads when combined with water being capable of releasing self-assembly structures comprising surfactant and celecoxib, the precipitation inhibitor at least predominantly not sharing the same volumes occupied by the non-ionic surfactant.
129. An oral celecoxib formulation, the formulation being a multiple minibead formulation wherein the minibeads comprise a hydrogel-forming polymer matrix in which are distributed celecoxib, a polyoxyethylated non-ionic surfactant and a precipitation inhibitor, the polymer forming a matrix in which the polyoxyethylated non-ionic surfactant is distributed as inclusions that comprise celecoxib and have a size of less than 25 μm, the precipitation inhibitor at least predominantly not sharing the same volumes occupied by the non-ionic surfactant.
130. The formulation of paragraph 128 or paragraph 129 that further includes the specific feature(s) recited in any of paragraphs 55 to 84 or in a combination thereof permitted by dependency.
131. A product for use in treating colorectal cancer or for use in treating colorectal inflammation or another gastrointestinal inflammatory condition, wherein the product is selected from the minibead of paragraph 122, the composition of any of paragraphs 123 to 126 and the formulation of any of paragraphs 127 to 130, in any such case being for use in treating colorectal cancer or another gastrointestinal inflammatory condition.
132. The product of paragraph 131 that is as further defined by any of paragraphs 90 to 99, or by a combination thereof permitted by dependency, or, as the case may be, is as further defined by any of paragraphs 104 to 107.
133. A product for use in inhibiting, reducing or delaying metastasis of a colorectal cancer, wherein the product is selected from the minibead of paragraph 52, the composition of any of paragraphs 53 to 81 and the formulation of any of paragraphs 82 to 88, the minibead of paragraph 122, the composition of any of paragraphs 123 to 126 and the formulation of any of paragraphs 127 to 130, in any such case being for use in inhibiting, reducing or delaying metastasis of a colorectal cancer.
134. The product of paragraph 133 that is as further defined by any of paragraphs 90 to 99 and 104 to 107, or by a combination thereof permitted by dependency.
135. A celecoxib product for use in treating colorectal cancer, wherein the product is selected from the minibead of paragraph 52, the composition of any of paragraphs 53 to 81 and the formulation of any of paragraphs 82 to 88, the minibead of paragraph 122, the composition of any of paragraphs 123 to 1269 and the formulation of any of paragraphs 127 to 130, wherein the product is for use in administering celecoxib at a daily dosage of less than 800 mg/day.
136. The product of paragraph 135 that is in unit dosage form, wherein the unit dosage form comprises less than 800 mg celecoxib.
137. The product of paragraph 135 or paragraph 136 that is for use in administering celecoxib at a daily dosage of less than 800 mg/day.
138. The product of paragraph 135 that is for use in administering celecoxib at a daily dosage of no more than 500 mg/day.
139. The product of paragraph 135 that is for use in administering celecoxib at a daily dosage of less than 200 mg/day.
140. The product of paragraph 135 that is for use in administering celecoxib at a daily dosage of no more than 100 mg/day.
141. The product of any of paragraphs 138 to 140 that is in unit dosage form, wherein the unit dosage form comprises celecoxib in an amount as referred to in the respective ones of paragraphs 138, 139 and 140.
142. A method for killing colorectal cancer cells in a subject, in which method apoptosis is favoured over necrosis, the method comprising administering to the subject a product selected from the minibead of paragraph 52, the composition of any of paragraphs 53 to 81 and the formulation of any of paragraphs 82 to 88, the minibead of paragraph 122, the composition of any of paragraphs 123 to 126 and the formulation of any of paragraphs 127 to 130.
143. A method for treating colorectal cancer in a subject, comprising administering to the subject a product selected from the minibead of paragraph 52, the composition of any of paragraphs 53 to 81 and the formulation of any of paragraphs 82 to 88, the minibead of paragraph 122, the composition of any of paragraphs 123 to 126 and the formulation of any of paragraphs 127 to 130.
144. The method of paragraph 143 that is for use in inhibiting, reducing or delaying the initiation and/or progression of colorectal cancer, or for use in causing regression of colorectal cancer, reducing the burden of colorectal cancer and/or inducing remission of colorectal cancer.
145. The method of paragraph 143 that is for use in inhibiting, reducing or delaying carcinogenesis of colorectal cancer.
146. The method of paragraph 143 that is for use in for use in causing regression of colorectal cancer, reducing the burden of colorectal cancer and/or inducing remission of colorectal cancer.
147. The method of paragraph 143 that is for use in inhibiting, reducing or delaying the progression of colorectal cancer.
148. The method of paragraph 143 that is for use in inhibiting, reducing or delaying metastasis of a colorectal cancer.
149. The method of any of paragraphs 143 to 148 wherein the colorectal cancer is a COX-2 positive adenocarcinoma.
150. The method of any of paragraphs 143 to 149 that is for use in administering celecoxib at a daily dosage of less than 800 mg/day.
151. The method of paragraph 150 that is for use in administering celecoxib at a daily dosage of no more than 500 mg/day.
152. The method of paragraph 150 that is for use in administering celecoxib at a daily dosage of less than 200 mg/day.
153. The method of paragraph 150 that is for use in administering celecoxib at a daily dosage of no more than 100 mg/day.
154. A method for treating colorectal inflammation or another gastrointestinal inflammatory condition in a subject, comprising administering to the subject a product selected from the minibead of paragraph 52, the composition of any of paragraphs 53 to 81 and the formulation of any of paragraphs 82 to 88, the minibead of paragraph 122, the composition of any of paragraphs 123 to 126 and the formulation of any of paragraphs 127 to 130.
155. The method of paragraph 154 that is for use in inhibiting, reducing or delaying the initiation and/or progression of the colorectal inflammation, or is for use in causing regression of or reducing the colorectal inflammation.
156. The method of paragraph 154 or paragraph 155 that is for use in administering celecoxib at a daily dosage of less than 200 mg/day.
157. The method of paragraph 156 that is for use in administering celecoxib at a daily dosage of no more than 100 mg/day.
158. The method of any of paragraphs 142 to 157 that is for administering to patients who suffer from intolerance to another NSAID or having a history of hypersensitivity to another NSAID.
159. The method of any of paragraphs 142 to 158 that further comprises administering another drug selected from anti-inflammatory drugs, immunosuppressants and drugs for the therapy or prophylaxis of cancer.
160. The method of paragraph 159 wherein said other drug is comprised in the product.
161. The method of paragraph 159 wherein said other drug is not in the product.
162. A method inhibiting, reducing or delaying metastasis of a colorectal cancer in a subject, comprising administering to the subject the minibead of paragraph 52, the composition of any of paragraphs 53 to 81 and the formulation of any of paragraphs 82 to 88, the minibead of paragraph 124, the composition of any of paragraphs 123 to 126 or the formulation of any of paragraphs 127 to 130.

### EXAMPLES

The following materials are used in examples:

| | |
|---|---|
| Kolliphor ® HS 15 (BASF) | Polyoxyl 15 hydroxystearate USP |
| | (Macrogol 15 hydroxystearate Ph. Eur) |
| Transcutol ® P (Gattefosse): | 2-(2-ethoxyethoxy)ethanol |
| Kolliphor ® EL (BASF) | Polyoxyl 35 Castor Oil USP |
| | (Macrogolglycerol Ricinoleate Ph. Eur.) |
| Miglyol ® 810N (Sasol) | Caprylic/Capric Triglyceride |
| Tween ® 20 (Merck) | Polysorbate 20 (Poly(oxyethylene) (20) |
| | sorbitan monolaurate) |
| Capryol ® 90 (Gattefosse). | Propylene glycol monocaprylate |
| | (type II) NF |
| Neosorb ® (Roquette, France) | Sorbitol |
| Surelease ® (Colorcon, USA) | Aqueous ethylcellulose dispersion |
| | (to provide ethylcellulose barrier |
| | coating) |

Kolliphor® HS 15was previously known as Solutol® HS-15. Kolliphor® EL was previously known as Cremophor® EL. The fatty acid content of Miglyol® 810N is 65-80% caprylic acid and 20-35% capric acid. Propylene glycol monocaprylate is a mixture of the propylene glycol monoesters and diesters of fatty acids composed predominately of caprylic acid; type II contains at least 90% monoesters and no more than 10% diesters.

Porcine gelatin was obtained from Nitta Gelatin, Japan, and sodium dodecyl sulfate (SDS) from Merck, Germany. . A sample of celecoxib active pharmaceutical ingredient (API) was kindly provided by Erregierre (Italy). The purity of the API was 99.6% based on the certificate of analysis provided by the supplier. All chemicals used for dissolution experiments, HPLC and UV testing were of laboratory grade.

### Example 1

### Solubilisation Studies

Solubilisation studies were performed using a wide range of vehicles consisting of oils, surfactants and co-solvents. The vehicles used were Kolliphor® HS 15, Transcutol® P, Kolliphor® EL, Miglyol® 810N, Tween® 20 and Capryol® 90.

A range of fluorescent dyes were sourced from Invitrogen

Solubilisation Measurements:

Celecoxib was added to measured quantities of the vehicles (excipients) in glass vials. These mixtures were stirred at room temperature on a magnetic stirrer; as an exception, the solubilisation measurements were performed at elevated temperatures in respect of vehicles which were solid at room temperature. Additional amounts of celecoxib were added to samples which remained transparent until maximum solubilisation was reached. The solubility of celecoxib in the liquid vehicles was recorded as the range between which the samples transgressed from transparent to cloudy, with the maximum solubilisation being within this range.

The vehicles were chosen based on a literature review of the various grades of vehicles available (Gibson, L., 2007. Lipid-based excipients for oral drug delivery In: Haus, D. J., (Eds.), Oral Lipid-Based Formulations—Enhancing the Bioavailability of Poorly Water-Soluble Drugs. New York, Informa Healthcare, 227-254; Cannon, J. B. and Long., M. A., 2008. Emulsions, micellar solutions, and lipid-based drug delivery systems for drug solubilisation and delivery—Part II: Oral applications In: Lui, R., (2^{nd} Edn.), Water-Insoluble Drug Formulation. Florida, Taylor and Francis, 227-254).

The solubility screening of celecoxib in a range of oils revealed that the best solubilisers of celecoxib within the oils tested were the propylene glycol esters. Capryol® 90 from Gattefosse had the best solubilising power with the solubility of celecoxib in Capryol® 90 determined to be 58-64 mg/g.

With respect to surfactants, the best results for the solubilisation of celecoxib were found with the hydrogenated castor oils, the polysorbates and a polyethylene glycol 660 hydroxystearate. Polyoxyl 40 hydrogenated castor oil (Kolliphor® EL), polysorbate 20 (Tween® 20) and polyethylene glycol 660 hydroxystearate (Kolliphor® HS-15) had solubilisation capacities for celecoxib of 264-330, 233-269 and 320-356 mg/g respectively.

The solubility of celecoxib was found to be high in all of the solvents tested with the exception of ethanol where the maximum solubility was determined to be between 100-125 mg/g. Celecoxib demonstrated exceptionally high solubility in 2-(2-ethoxyethoxy)ethanol (Transcutol® P) where the solubility was measured to be 345-390 mg/g.

### Example 2

### In-vitro Dissolution Testing of Celecoxib Liquid Formulations

Liquid formulations containing celecoxib dissolved in combinations of oils/surfactants/co-solvents were prepared on the basis of the results of the screening studies of Example 1. In-vitro dissolution testing was performed on these formulations to assess their performance. In-vitro dissolution testing was also performed on the celecoxib Active Pharmaceutical Ingredient (API) and the marketed product Celebrex®. Unlike the majority of dosage forms (in particular other oral dosage forms) in which the API is present in a solid format (e.g., tablets and granules), the drug in lipid based dosage forms (e.g., soft gelatin capsules) is usually pre-dissolved, therefore the standard dissolution test is a measure of how well the drug disperses or releases into the chosen media rather than a measure of how the drug dissolves. When designing a dissolution experiment for the testing of lipid based dosage forms, the contents of the dissolution medium are important, this is particularly important when trying to mimic in-vivo conditions. For example, in the gastrointestinal tract, dispersion of the formulation will occur via emulsification in the stomach and small intestine (Gibson, 2007, see above) and therefore it is important to mimic the components of the intestinal fluids to simulate this emulsification process. An alternative approach to the dissolution testing of lipid-based dosage forms (the approach employed here) is to test the dosage forms in simple aqueous media such as Purified Water (PW), Simulated Gastric Fluid (SGF—pH 1.2) and Simulated Intestinal Fluid (SIF—pH 6.8) with the primary purpose of acting as a screening tool to identify superior formulations. This approach adopts a ‘worst case approach’ in which it is postulated that positive results for the dissolution of celecoxib in these media may indicate an enhanced effect under in-vivo conditions.

Dissolution studies revealed that the release of celecoxib was independent of the pH of the dissolution medium and, therefore, purified water was used as the dissolution medium of choice. This result is consistent with a European Medicines Agency (EMA) discussion paper on the approval of Onsenal® (a celecoxib product) where it is stated that celecoxib is weakly acidic (pKa is near 11) leaving the molecule uncharged at physiological pH's and therefore practically insoluble in aqueous buffers at a range of physiological pH's (EMA Scientific Discussion on Onsenal, 2004, available at http://www.ema.europa.eu/docs/en_GB/document_library/EPAR_Scientific_Discussion/human/000466/WC500044630.pdf). In the same discussion paper it is stated that it was necessary to use a dissolution method with a non-physiological pH of 12 and 1% sodium lauryl sulphate to obtain sink conditions for celecoxib and that, due to the very low solubility of the drug in physiologically relevant media, no clinically meaningful in-vitro in-vivo correlation could be established. The same dissolution medium is employed for the regulatory dissolution testing of Celebrex® (FDA dissolution methods database, 2013).

In-vitro Dissolution Testing:

Dissolution of celecoxib formulations was performed (n=3) at 37° C. in Simulated Intestinal Fluid (SIF—pH 6.8), Simulated Gastric Fluid (SGF-pH 1.2) and Purified Water (PW). The protocols for the SIF and the SGF were taken from the United States Pharmacopoeia (USP 33—NF 28, 2010). Dissolution experiments were carried out using either a Varian/Vankel VK7010 dissolution apparatus (VanKel, USA) or a Distek Evolution 6300 (Distek, USA) equipped with standard glass vessels and USP type II paddles. Paddle rotating speed in all experiments was 75 rpm. Formulations containing 50 mg of dissolved celecoxib were weighed and added to 1000 mL of the relevant dissolution medium (SIF, SGF or PW). At specified times 1.8 mL samples were withdrawn, filtered through a 70 μm pore filter (QLA, USA) and analysed using either a high performance liquid chromatography (HPLC) method or an ultraviolet (UV) spectrophotometric method analysis. From the absorbance value (UV method) and the area under the curve (HPLC method), the % of drug released at particular time points was calculated.

HPLC and UV Analysis:

The HPLC method for the analysis of the dissolution and assay samples was adapted from Saha et al. (2002). The HPLC column used was a reverse phase 4.6×250 mm lnertsil® C 8 column (lnertsil, The Netherlands) with 5 μm particles. The mobile phase was acetonitrile:water (65:35). The isocratic method used a flow rate of 1.25 ml/min and ultraviolet (UV) detection at 230 nm. The injection volume was 20μl and the retention time was 8 minutes. The HPLC apparatus that was used for the analysis were Thermo Finnigan and Waters HPLC system (and associated Chromquest and Empower software). The UV method for the analysis of the dissolution samples was also adapted from Saha et al. (Saha et al. 2002). The spectrophotometer used was a A Genesys 10 series UV-visible spectrophotometer (Thermo Electron Corporation, USA).Absorbance was read at a wavelength of 251 nm.

Three liquid formulations, formulations A, B and C (Table 1) were prepared by dissolving celecoxib in mixtures of Kolliphor® HS-15/Miglyol® 810N, Tween® 20/Miglyol® 810N and Transcutol® P/Tween® 20/Miglyol® 810N respectively.

TABLE 1

| | Formulation Composition | | |
|---|---|---|---|
| | | % | Oils/Surfactants/ |
| | % | Gelatine | Co-Solvents (Ratios) |
| Formulation | Celecoxib | Phase | (% of Formulation) |
| Formulation A | 7 | — | Miglyol ® 810N:Kolliphor ® |
| (liquid) | | | HS-15 (35:65) (93) |
| Formulation B | 7 | — | Miglyol ® 810N:Tween ® 20 |
| (liquid) | | | (35:65) (93) |
| Formulation C | 9 | — | Miglyol ® 810N:Transcutol ® |
| (liquid) | | | P:Tween ® 20 (25:25:50) (91) |

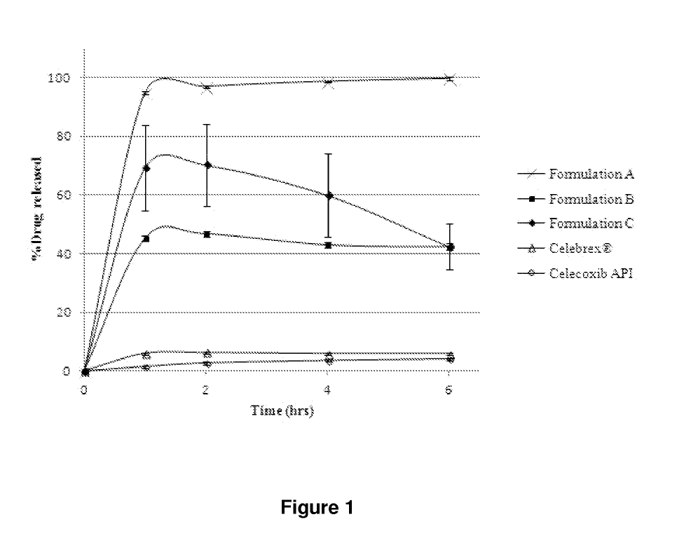

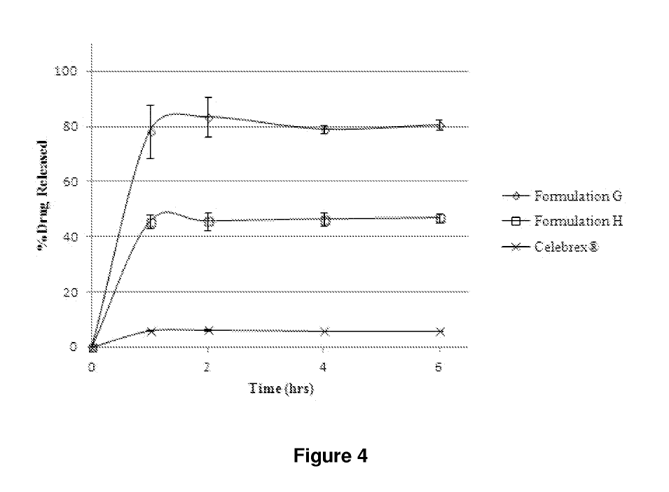

Set quantities of the formulations equating to 50 mg of celecoxib were filled into empty hard gelatin capsules and were added to the dissolution medium (PW). The release of formulation A was 94.87% after 1 hour and was vastly superior to that of the celecoxib API and the Celebrex® capsules (FIG. 1). The extent of dissolution was comparable with results presented by Shakeel and Faisal, in which they showed that nano-emulsions were promising vehicles for solubility and dissolution enhancement of celecoxib (Shakeel, F., and Faisal, M. S. Nanoemulsion: A promising tool for solubility and dissolution enhancement of celecoxib. Pharmaceutical Development and Technology, 2010; 15(1): 53-56.). This test demonstrated that the dissolution of celecoxib could be increased by formulating the drug in a lipophilic format. It is acknowledged that Celebrex® would be expected to perform better in-vivo than that illustrated by the data presented in FIG. 1 (and later in FIG. 4) due to the presence of dissolution enhancing entities in-vivo in the GI tract (e.g., bile salts) however these entities would also be expected to enhance or maintain the % release of the celecoxib lipid formulations presented. Such a correlation was seen observed in a study by Subramanian et. al., where the enhanced dissolution of a celecoxib SMEDDS formulation compared to that of another commercial celecoxib powder filled capsule (Celact®) resulted in a corresponding increase in bioavailabilty (Subramanian et al., Biol Pharm Bull, 2004: 27(12): 1993-9).

Although the celecoxib was completely dissolved in formulations B and C (FIG. 1), this was not sufficient to enable the drug to fully disperse within the dissolution medium or to avoid the drug precipitating over the course of the dissolution test. In the case of formulation B, it is likely that the balance of the hydrophobic and hydrophilic components in the formulation was not sufficient ensure full dispersal of the drug, whilst in the case of formulation C the balance was not sufficient to prevent the Transcutol solvent dissipating into the aqueous medium, thus lowering the solubility of the drug and leading to its precipitation.

The variation in the dissolution performance for formulation C is also notable in comparison to formulations A and B where the standard deviation for all timepoints was less than 1%, which reinforces the hypothesis that the drug precipitated in the case of formulation C.

### Example 3

### Celecoxib Minibead Formulations

The results reported above constituted a step towards the development of an improved oral lipophilic drug delivery system for celecoxib. Although the liquid formulations described demonstrated improved solubility and dissolution of the drug, the requirement for the inclusion of high levels of surfactant in these formulations precluded their incorporation into conventional oral dosage forms such as soft gelatin capsules or microcaspules, due to interactions between the inner capsule contents and the capsule shell. This is a challenge also posed to the nanoemulsion formulation presented by Shakeel and Faisal (Shakeel and Faisal , 2010, see above), as nanoemulsions with a high water content have been shown to be unsuitable for incorporation into soft gelatin, hard gelatin or hydroxypropylmethylcellulose capsules for oral delivery due to the high water content of these type of formulations promoting hydrolysis and/or precipitation of certain drugs on long-term storage, which ultimately affect their utility in oral delivery (Date et al., Nanomedicine, 2010: 5 (10): 1595-1616).

Based on current marketed technologies, the only suitable mode of administration of an emulsion would be as an oral solution, however oral solutions have an inherent disadvantage in that they are not amenable to further processing to allow for targeted or sustained delivery (e.g., for colonic delivery). It is acknowledged that Self Emulsifying Drug Delivery Systems (SEDDS) and Self Micro-Emulsifying Drug Delivery Systems (SMEDDS) are suitable for incorporation into soft gelatine capsules and a number of celecoxib SEDDS/SMEDDS have been reported (Subramanian et al., Biol Pharm Bull, 2004: 27(12): 1993-9; and Song et al., Arch. Pharm. Res, 2013; 36: 69-78). Although the delivery of celecoxib SEDDS/SMEDDS have many advantages including improved solubility and bioavailability, one of their primary disadvantages is that they are currently delivered as single dosage units (e.g., in soft gelatine capsules). There are a number of advantages of delivering drugs in a multiparticulate format including: predicative gastric emptying, reduced risk of local irritation, reduced risk of systemic toxicity (reduced risk of dose dumping) and increased bioavailability (Asghar and Chandra, J Pharm Pharmaceut Sci, 2006; 9(3): 327-338). Multiparticulates also present advantages for populations who have difficulty swallowing, namely geriatrics and paediatrics. In the case of celecoxib a multiparticulate formulation is particularly desirable from the perspective of delivering the drug to the colon in the chemoprevenative treatment of colorectal cancer and in the avoidance of GI irritation as a result of dose dumping. Multiparticulate formulations containing celecoxib SMEDDS/SEDDS have been attempted, however entrapment efficiencies greater than 85% have not been achieved (Homar et al., Journal of Microencapsulation, 2009: 26(6): 479-484),

The next step of this study was to develop a multiparticulate technology into which these formulations could be efficiently encapsulated. It was proposed that gelatin based minibeads could be manufactured in which the liquid formulation would be entrapped with a gelatin matrix. Minibeads containing celecoxib were prepared via the manual drop in oil method described. In some of the formulations other components were added to the gelatin phase to prevent precipitation of drug during manufacturing and also to help maintain the minibead structure. Optimised minibeads, that is minibeads that were robust and were amenable to further processing were evaluated with respect to their entrapment efficiency and in-vitro dissolution.

Preparation of Minibeads:

Minibeads were prepared using vehicles detailed in Example 1 in combination with porcine gelatin (Nitta Gelatin), sorbitol (Neosorb®-Roquette), SDS and Opadry® White 20A28380 (Colorcon; a mixture of HPC, HPMC, talc and TiO₂). Minibead Formulations D to H are detailed in Table 2 below. The dry ingredients of Formulations D to H and also of a Formulation K are specified after Table 2.

Minibeads containing celecoxib were prepared via a manual drop in oil method. The minibeads were manufactured by combining the surfactant phase (drug dissolved in vehicles) with a gelatin phase (mixture of gelatin, water, sorbitol and SDS) and mixing at approximately 60° C. Droplets of the mixture were then allowed to fall into a bath of cooling/hardening oil (Miglyol® 810N) at approximately 10° C. As the mixture droplets fall in air, coacervation occurs to form a coacervate suspension in which droplets of the surfactant phase are surrounded by a layer of gelatine (multiple coacervates exist within a single minibead). The dropping of the minibead into a cooling/hardening bath allows the minibeads to harden before being recovered for drying. The minibead formation occurs in air prior to impaction with the cooling oil, thereby preventing interaction of the cooling oil with the internal surfactant phase droplets due to the presence of the layer of gelatin at the surface of the coacervates and hence the minibead. A range of oils was investigated (olive oil, mineral oil and other medium chain triglycerides) for use as the cooling/hardening oil in the production of minibeads. Miglyol® 810N was selected as it was observed to perform optimally in the production of spherical minibeads, it has been shown to be stable against oxidation and is listed on the FDA's inactive ingredient database. The minibeads were then air dried for 24 hr (over this time the water in the formulation evaporated). In some of the formulations other components were added to the gelatin phase to prevent precipitation of drug during manufacturing and also to help maintain the minibead structure.

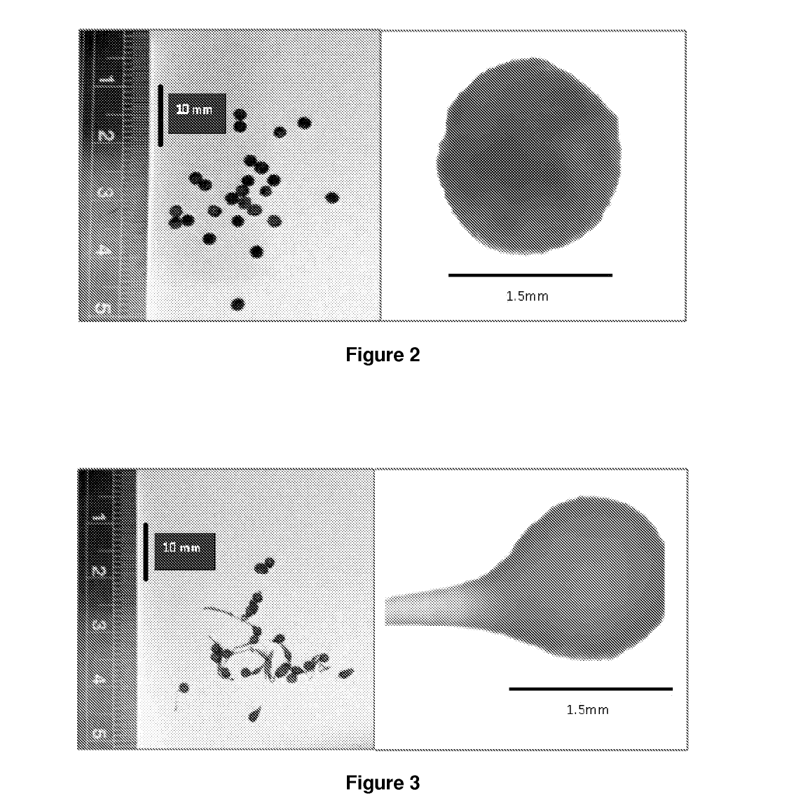

One of the initial minibead formulations produced was based on formulation A (see Table 1 above). Formulation D (Table 2) contained celecoxib dissolved in an surfactant phase consisting of Kolliphor® HS 15 and Miglyol® 810N, which translated into a final drug loading of 2% w/w of celecoxib in the minibead. The emulsion produced from the combination of the non-ionic surfactant and gelatin phase components was transparent, suggesting that it was a micellar solution. The resultant minibeads were roughly spherical and robust and became opaque upon drying. Photographs of minibeads from this batch of minibeads are shown in FIG. 2. The shape of these minibeads compared favorably with dried celecoxib microcapsules in a previous report by Homar et al., in which the microcapsules produced were irregular in shape (Homar et al., 2009, see above).

TABLE 2

| | Formulation Composition | | |
|---|---|---|---|
| | | % | Oils/Surfactants/ |
| | % | Gelatine | Co-Solvents (Ratios) |
| Formulation | Celecoxib | Phase | (% of Formulation) |
| Formulation D | 2.3 | 62 | Miglyol ® 810N:Kolliphor ® |
| (bead) | | | HS-15 (35:65) (36) |
| Formulation E | 5.8 | 61 | Miglyol ® 810N:Kolliphor ® |
| (bead) | | | HS-15 (35:65) (33) |
| Formulation F | 8.3 | 44 | Kolliphor ® HS-15 |
| (bead) | | | (48) |
| Formulation G | 6.1 | 35 | Kolliphor HS-15 ®:SDS (93:7) |
| (bead) | | | (59) |
| Formulation H | 6.3 | 37 | Kolliphor HS-15 ® |
| (bead) | | | (57) |

Percentages in Table 2 are percent by weight based on the dry weight of the formulation, i.e. percent by weight of the constituents other than water.

The ingredients of Formulations D to H and of Formulation K are presented in detail below:

Formulation D

| Component | w/w % |
|---|---|
| Celecoxib | 2.3 |
| Kolliphor HS 15 | 23.0 |
| Miglyol 810N | 13.1 |
| Gelatin | 55.4 |
| D-Sorbitol | 6.2 |

Surfactant phase to gelatine phase ratio on a wet basis: 1:8
Kolliphor : Water Ratio: 1:10.7

Formulation E

| Component | w/w % |
|---|---|
| Celecoxib | 5.8 |
| Kolliphor HS 15 | 21.9 |
| Miglyol 810N | 10.8 |
| Gelatin | 55.4 |
| D-Sorbitol | 6.1 |

Surfactant phase to gelatine phase ratio on a wet basis: 1:8
Kolliphor : Water Ratio: 1:11.2

Formulation F

| Component | w/w % |
|---|---|
| Celecoxib | 8.3 |
| Kolliphor HS 15 | 47.2 |
| Gelatin | 40.0 |
| D-Sorbitol | 4.5 |

Surfactant phase to gelatine phase ratio on a wet basis: 1:4
Kolliphor : Water Ratio: 1:3.8

Formulation G

| Component | w/w % |
|---|---|
| Celecoxib | 6.1 |
| Kolliphor HS-15 | 54.6 |
| Gelatin | 31.6 |
| SDS | 4.2 |
| D-Sorbitol | 3.5 |

Surfactant phase to gelatine phase ratio on a wet basis: 1:3
Kolliphor : Water Ratio: 1:2.6

Formulation H

| Component | w/w % |
|---|---|
| Celecoxib | 6.3 |
| Kolliphor HS-15 | 56.8 |
| Gelatin | 33.1 |
| D-Sorbitol | 3.8 |

Surfactant phase to gelatine phase ratio on a wet basis: 1:3
Kolliphor : Water Ratio: 1:2.7

Formulation K

| Component | w/w % |
|---|---|
| Celecoxib | 5.5 |
| Kolliphor HS-15 | 49.6 |
| Gelatin | 39.3 |
| Opadry | 1.2 |
| D-Sorbitol | 4.4 |

“Opadry” is Opadry® White 20A28380, used as a precipitation inhibitor.
Surfactant phase to gelatine phase ratio on a wet basis: 1:4
Kolliphor : Water Ratio: 1:3.5

Although formulation D was shown to be a robust formulation, the final drug loading was not sufficient for it to be considered as a viable product. Similar formulations were produced in which the drug loading was increased to 9-10% w/w, however precipitation was evident for these formulations on contact of the surfactant phase with the gelatin phase. Formulation E (Table 1) was produced on the basis of the success of formulation D, with the drug loading increased to 6%. There was evidence of slight precipitation in the case of formulation E. It was concluded that the loading of celecoxib played a crucial role with respect to precipitation. It was also observed that the shape of the resultant minibeads was not amenable to further processing as there was tailing evident (FIG. 3). Dissipation of the emulsion into the cooling oil was observed during the manufacture of these minibeads. It was proposed that the inclusion of Miglyol® 810N in the formulation may have caused this dissipation. Miglyol® 810N was employed as the cooling oil, therefore it was proposed that the Miglyol® 810N in the formulation had a high affinity for the cooling oil resulting in dissipation of the formulation. This may also have contributed to the poor minibead shape. This formulation highlighted the requirement to prevent precipitation and the need to tailor the formulation to maintain minibead shape.

Two successful approaches were adopted to combat precipitation. The first approach involved modifying the concentration of the gelatin phase in the formulation. Given the observation that increasing the concentration of celecoxib in the surfactant phase generally led to increased precipitation, the possibility of reducing the concentration of the gelatin phase was investigated as an alternative method of increasing the loading of celecoxib. In all of the formulations presented above, the concentration of the gelatin phase contributed to approximately 60% w/w dry weight of the entire formulation. Formulation F (Table 1) was produced in which the concentration of the gelatin phase in the resultant minibeads was 44% w/w (surfactant phase contained celecoxib dissolved in Kolliphor® HS-15). Formulation F had a drug loading of 8%. There was some slight precipitation evident over time; however this was a major advance on the previous formulations produced.

It was proposed that improvement with respect to the precipitation of the drug was as a result of the reduced number of water molecules (in the gelatin phase) available for interaction with the drug. It was also shown that formulations such as formulation F were easily converted into robust minibeads. A second approach adopted to prevent precipitation was to include SDS (sodium dodecyl sulfate).

All formulations containing SDS comprised a gelatin phase in the region of 30-40% w/w. In an effort to eradicate the slight precipitation observed for formulation F, SDS was included at different concentrations in a range of similar formulations. An optimal formulation (Formulation G—Table 1) was produced in which precipitation was completely absent.

Evaluation of Entrapment Efficiency:

The amount of incorporated celecoxib was determined in the optimised minibead formulations produced. A quantity of minibeads (n=3) with a theoretical potency between 5 mg and 50 mg (depending on the quantity of sample available) were sonicated for 2 hr in a mixture of acetonitrile:water 65:35 (HPLC method) or acetonitrile:phosphate buffer 50:50 (UV method) to extract the drug from the minibeads. The resultant solution was passed through a 0.45 μm filter prior to absorbance analysis. Where required the samples were diluted prior to analysis. The concentration of celecoxib was determined by absorbance measurements at 230 or 251 nm via the HPLC or UV analysis methods described above (see Example 2). Celecoxib content (%) was calculated as the amount of determined celecoxib with respect to the total mass of dried minibeads. The entrapment efficiency (%) of celecoxib was expressed as a percentage of the determined celecoxib with respect to the total amount of celecoxib used in the preparation of the minibeads.

The mean entrapment efficiency of formulation G was 98.07% (relative standard deviation 0.657%-Table 2). This compares favorably to results presented by Homar et al. where entrapment ranged between 60-82% for celecoxib microcapsules (Homar et al., 2009, se above) The high % of entrapment efficiency achieved demonstrates that the input formulation composition was retained following minibead production as the loss of any of the surfactant phase components to the cooling oil would result in a corresponding loss of drug and thereby a lower % of entrapment. The drug loading of this formulation was 6.0% w/w (a level considered to be commercially viable). The formulation was transparent (the original liquid) which suggests that it was a micellar solution/micellar solution. Dissolution analysis was performed on 50 mg doses of this formulation and is presented in FIG. 4. The dissolution profile of the marketed product Celebrex® is included again for comparison purposes. The dissolution media employed was purified water. The reader is reminded that in the case of the Celebrex® (marketed celecoxib product), the maximum amount (%) of celecoxib released in purified water was 6%. In contrast the maximum release for this optimised formulation was 80%, this data represents an increase in the release of celecoxib from microcapsules in a similar way to that reported by Homar and colleagues (Homar et al., Journal of Microencapsulation, 2007; 24(7): 621-633 and Homar et al., Journal of Microencapsulation, 2009: 26(6): 479-484). In the latter study, celecoxib microcapsule formulations with a maximum release ranging from 9-16% were reported (the dissolution experiments were performed with 3 mg doses). In the 2007 study (Homar et al., 2007), celecoxib microcapsule formulations with a maximum release in the range of 60-80% were reported however the dissolution media employed contained surfactant (2% Tween 80) to facilitate the release of the drug and the dissolution experiments were performed on 3 mg doses. This data illustrates that the novel minibead approach presented here in this paper is advantageous to the microcapsule approach presented by Homar et al.

Another formulation in which no SDS was included (Formulation H—Table 1) was also tested to evaluate the impact of SDS with respect to in-vitro dissolution (in addition to its impact with respect to precipitation). The removal of SDS resulted in a dramatic decrease in the release, with a maximum of 46% release for this formulation. The mean entrapment efficiency of formulation H was 96.07% (relative standard deviation 0.421%—Table 2)

### Example 4

### In-Vitro Dissolution Profile of Uncoated Minibeads of Formulations G, H and K

The in-vitro dissolution profiles of a sample of each of the minibead formulations G, H and K and Celebrex® were measured in purified water at 37° C. Dissolution experiments were carried out using either a Varian/Vankel VK7010 dissolution apparatus (VanKel, USA) or a Distek Evolution 6300 (Distek, USA) equipped with standard glass vessels and USP type II paddles. Paddle rotating speed in all experiments was 75 rpm. At specified times 1.8 mL samples were withdrawn, filtered through a 70 μm pore filter (QLA, USA) and analysed using either a high performance liquid chromatography (HPLC) method. From the area under the curve (HPLC method^{[18]}), the % of drug released at particular time points was calculated. The results are shown in Table 3 below.

TABLE 3

| | Formulation | Formulation | Formulation | | Celecoxib |
|---|---|---|---|---|---|
| Time | G | H | K | Celebrex | API |
| (hrs) | % Released | | | | |
| 0 | 0 | 0 | 0 | 0 | 0 |
| 1 | 78 | 45 | 69 | 6 | 1 |
| 2 | 83 | 46 | 68 | 6 | 3 |
| 4 | 79 | 46 | 68 | 6 | 4 |
| 6 | 81 | 47 | 69 | 6 | 4 |

### Example 5

### Celecoxib Minibead Characterisation

### Minibead Characterization Using Light Microscopy

Shape and surface morphology of freshly prepared and dried minibeads were observed under a Nikon Eclipse Ti optical microscope mounted with a digital camera. Pictures were taken of sliced sections of dried minibeads. Thin films of selected formulations were also prepared and viewed under the microscope in an effort to understand the internal structure of the minibeads. A number of fluorescent dyes were incorporated into some of these formulations in an attempt to distinguish between the non-ionic surfactant and water phases of the formulations.

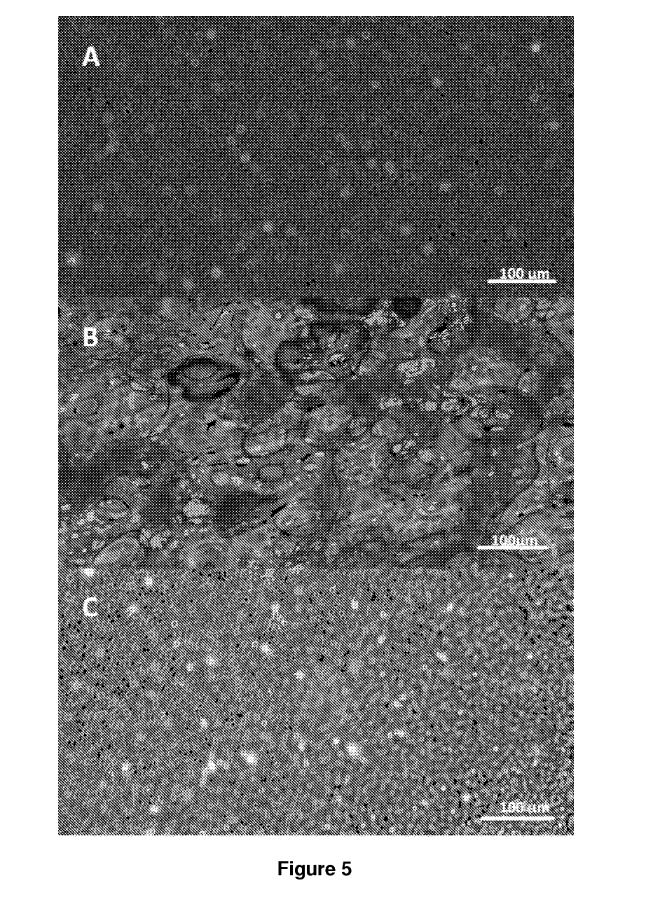

In order to compare and understand the performance of the formulations described, it was necessary to characterise the internal structure of these formulations. Initial attempts at image analysis were performed on slices of minibeads however this was found to be a destructive method (the integrity of the minibead was compromised as a result of the slicing process). As an alternative, thin films of the minibead formulations were prepared. A number of fluorescent dyes (Nile Red, Bodipy 505/515, Sudan Orange, Dextran Cascade Blue, Dextran Rhodamine Green and Dextran Alexa Fluor 546) were incorporated into some of these formulations in an attempt to distinguish between the surfactant and water phases of the formulations. In order to understand the structure of optimised formulations, a number of formulations were prepared to act as controls or to assess the impact of the addition and removal of certain formulation components (e.g., SDS). Formulation H was prepared to assess the impact of removing SDS from the formulation. Photographs of thin films of each of these formulations were taken using a Nikon Eclipse Ti inverted microscope (FIG. 5). A combination of Dextran Alexa Fluor 546 (soluble in the gelatin phase) and Nile Red (soluble in the surfactant phase) were found to be the best dyes for distinguishing between the two phases. The microscope was predominantly used in its light microscope setting as opposed to fluorescent light (greater clarity was observed using the light microscope).

In FIG. 5A, the structure of a film of a gelatin formulation is represented. This formulation acted as a control for comparison with the other formulations. The gelatin formulation appeared to have a consistent matrix appearance in which there were distinct regions (light and dark regions). This was not unexpected as gelatin is a polydisperse system comprising different lengths of protein chains that in turn consist of long hydrophobic chain segments and short hydrophilic segments (i.e., it has amphiphilic properties). In FIG. 5B, the structure of formulation H is represented. Photograph 5B illustrated a system in which there was evidence of the presence of large polydisperse surfactant phase droplets/vesicles. In FIG. 5C, the structure of formulation G is represented. The image in FIG. 5C was similar to that for the gelatin control in FIG. 5A. It is proposed that the surfactant phase droplets/vesicles present in formulation G were too small to be visible (i.e., mixed micelles could be present). This is an important finding as it suggests that formulation G was a micellar solution/micellar solution (which corresponds with the fact that the liquid formulation was transparent) in comparison to other formulations such as formulation H which were opaque. It is suggested that the superior in-vitro dissolution performance of formulations such as formulation G in comparison to formulations which contained none or less surfactant were related to the droplet size of these formulations.

Examples 1 to 4 show that the optimal minibead formulation produced (formulation G) was demonstrated to have a superior dissolution performance than that of the marketed celecoxib product Celebrex®. Formulation G is believed to be a micellar solution/micellar solution based on the transparent appearance of the original liquid and image analysis of the resultant minibeads. This is important as the performance of micellar solution formulations in-vivo have, in some cases, been shown to be superior (better and more consistent absorption, less impact of food effects etc.) in comparison to equivalent emulsion formulations. An example of this is improved performance of the Novartis micellar solution Cyclosporin preparation Neoral® in contrast to equivalent emulsion formulation Sandimmune® (Mueller et al., 1994).

Despite the surmounting evidence that celecoxib is a potentially useful anticancer drug for the prevention or treatment of colorectal cancer, a number of questions regarding the safety and efficacy of the drug remain (Gonzalez-Angulo and Fuloria, Ochsner J., 2002; 4(3): 176-179). The safety concerns relating to celecoxib relate to the serious gastrointestinal (GI) and cardiovascular (CV) side effects associated with Celebrex®. The CV side effects of celecoxib are dose related and it is proposed that the GI side effects are as a result of local irritation and therefore are related to the presentation of the current dosage form (a powder filled capsule).

### Example 6

### Investigation of Potential Precipitation Inhibitors

Further potential precipitation inhibitors were investigated with the following results shown in Table 4. It is believed that it might have been possible to disperse the HPC if this had been finely milled.

TABLE 4

| Precipitation Inhibitors | Effect |
|---|---|
| Vitamin E TPGS | Effective precipitation inhibitor but negative |
| | impact on bead formation |
| Vitamin E Acetate | Effective precipitation inhibitor but negative |
| | impact on bead formation |
| Opadry II (PVA based) | Potential for preventing precipitation but |
| | mixture became too viscous |
| HPMC (Methocel E15) | Reduced precipitation but beads became |
| | difficult to process (viscosity) |
| HPC | Difficult to disperse the HPC in the mixture |
| | and became viscous |
| Pluronic ® F127 | Not effective at preventing precipitation |
| Hypromellose (HPMC) | This sample not effective at preventing |
| | precipitation |
| PVA | Not effective at preventing precipitation |

### Examples 7 to 13

### In Vitro Characterisation of Microbead Formulation

In-vitro model materials McCoy's 5A modified medium, L-glutamine, 10% FBS (fetal bovine serum), 1% penicillin/streptomycin, molecular grade DMSO (all Sigma Aldrich, USA), VybrantMTT assay kit (Invitrogen, USA) and Annexin-PE Apoptosis Detection Kit (BD Biosciences, USA).

Liquid formulations containing celecoxib were manually prepared as previously described in Example 1.

Minibeads containing celecoxib were manually prepared as previously described in Example 3. Maximum batch sizes equated to approximately 1 g.

Statistical analysis was performed using GraphPad Prism (La Jolla, USA). Results are presented as mean±SEM (3-10). Differences, indicated as two-tailed P values, were considered significant when P<0.05 as assessed by unpaired Student's t test.

### Example 7

### Automated Preparation of Minibeads

A vibrating nozzle, jet break-up encapsulator (Inotech model IE-50 R, Switzerland) equipped with a 1 mm diameter single nozzle, an air pressure solution delivery system and a temperature controlled nozzle jacket was used to prepare minibeads. The details of the formulation preparation, the cooling oil used and the cooling oil temperature were as previously described in Example 3. The nozzle jacket was set to 70° C. The minibeads were produced at a solution flow rate of 10 g/min. Minibeads were removed from the cooling oil and were allowed to dry at ambient temperature for 18-24 hours before being sieved between 1-1.3 mm on 100 mm stainless steel sieves (Retsch, Germany). Batch sizes in the region of 50 g were produced.

### Example 8

### Fluid Bed Coating of Minibeads

Minibead Formulations G1 to G6 are detailed in Table 5 below. The dry ingredients of Formulations G1 to G6 are specified after Table 5.

Coating solutions were prepared using Surelease® and purified water as per the manufacturer's instructions. The starting weight of minibeads for coating was approximately 6 g. For the coating of manually produced batches of minibeads, due to the limited availability of material, approximately 5 g of coloured placebo minibeads were added to 1 g of active minibeads to enable a batch size that was sufficient for coating. An MFL01 fluid bed system (Vector Corporation, USA) equipped with a Wurster insert was used for coating. Minibeads were coated at an inlet air temperature setting of 65° C. and a product temperature of 40° C. The volume of fluidizing air was maintained at 179 LPM (litres per minute) to ensure optimum fluidizing of microcapsules. A nozzle air pressure of 1.7 bar and a solution flow rate of 3-4 g/min was applied. At the end of the coating process, the coated minibeads were dried for 5 minutes in the fluid bed system at a product temperature of 40° C. The weight gain of the minibeads was calculated based on the starting (pre-coating) and end (post-coating) weights of the minibeads.

TABLE 5

| | | | |
|---|---|---|---|
| | | % | |
| | % | Gelatine | Surfactants (Ratios) |
| Formulation | Celecoxib | Phase | (% of Formulation) |

| | |
|---|---|
| Formulation | Formulation G coated with 5% weight gain of Surelease ® |
| G1 (coated | |
| minibead) | |
| Formulation | Formulation G coated with 8% weight gain of Surelease ® |
| G2 | |
| (coated | |
| minibead) | |
| Formulation | Formulation G coated with 17% weight gain of Surelease ® |
| G3 (coated | |
| minibead) | |
| Formulation | Formulation G coated with 20% weight gain of Surelease ® |
| G4 (coated | |
| minibead) | |
| Formulation | Formulation G coated with 27% weight gain of Surelease ® |
| G5 (coated | |
| minibead) | |
| Formulation | Formulation G coated with 32% weight gain of Surelease ® |
| G6 (coated | |
| minibead) | |

| | |
|---|---|
| Formulation G1 | Formulation G2 |

| Component | w/w % | Component | w/w % |
|---|---|---|---|
| Celecoxib | 5.8 | Celecoxib | 5.6 |
| Kolliphor HS-15 | 52.0 | Kolliphor HS-15 | 50.5 |
| Gelatin | 30.1 | Gelatin | 29.3 |
| SDS | 4.0 | SDS | 3.9 |
| D-Sorbitol | 3.3 | D-Sorbitol | 3.2 |
| Surelease | 4.8 | Surelease | 7.4 |

| | |
|---|---|
| Formulation G3 | Formulation G4 |

| Component | w/w % | Component | w/w % |
|---|---|---|---|
| Celecoxib | 5.2 | Celecoxib | 5.1 |
| Kolliphor HS-15 | 46.6 | Kolliphor HS-15 | 45.5 |
| Gelatin | 27.0 | Gelatin | 26.3 |
| SDS | 3.6 | SDS | 3.5 |
| D-Sorbitol | 3.0 | D-Sorbitol | 2.9 |
| Surelease | 14.6 | Surelease | 16.7 |

| | |
|---|---|
| Formulation G5 | Formulation G6 |

| Component | w/w % | Component | w/w % |
|---|---|---|---|
| Celecoxib | 4.8 | Celecoxib | 4.6 |
| Kolliphor HS-15 | 43.0 | Kolliphor HS-15 | 41.3 |
| Gelatin | 24.9 | Gelatin | 23.9 |
| SDS | 3.3 | SDS | 3.2 |
| D-Sorbitol | 2.8 | D-Sorbitol | 2.7 |
| Surelease | 21.2 | Surelease | 24.3 |

### Example 9

### In-Vitro Dissolution Profile of Coated Minibeads of Having Different % Weight Gain and Celebrex®

Dissolution of coated celecoxib minibeads G1 to G6 was performed (n=3) at 37° C. in a two step enteric media (2 hrs in 0.1 M HCl (750 ml) followed by 22 hrs in phosphate buffer, pH 6.8 (1000 ml). Dissolution experiments were carried out using either a Varian/Vankel VK7010 dissolution apparatus (VanKel, USA) or a Distek Evolution 6300 (Distek, USA) equipped with standard glass vessels and USP type II paddles. Paddle rotating speed in all experiments was 75 rpm. Minibeads equating to 25 mg of celecoxib were weighed and added to the enteric media. At specified times 1.8 mL samples were withdrawn, filtered through a 70 μm pore filter (QLA, USA) and analysed using either a high performance liquid chromatography (HPLC) method. From the area under the curve (HPLC method^{[18]}), the % of drug released at particular time points was calculated. The % of drug release was adjusted to take into account the % entrapment efficiency of the formulation. The results are shown in Table 6.

TABLE 6

| Time | G1 | G2 | G3 | G4 | G5 | G6 | Celebrex |
|---|---|---|---|---|---|---|---|
| (hrs) | % Released | | | | | | |
| 0 | 0 | 0 | 0 | 0 | 0 | 0 | 0 |
| 1 | 36 | 20 | 3 | 0 | 0 | 0 | 8 |
| 2 | 71 | 40 | 34 | 0 | 0 | 0 | 6 |
| 4 | 86 | 63 | 61 | 44 | 30 | 12 | 6 |
| 6 | 87 | 71 | 69 | 73 | 56 | 54 | 6 |
| 12 | 85 | 79 | 68 | 71 | 69 | 71 | 6 |
| 24 | 85 | 79 | 65 | 66 | 67 | 66 | 7 |

### Example 10

### Effects of Celecoxib Formulations on the Proliferation of HT29 Cells

HT29 is a COX-2 positive human colorectal adenocarcinoma cell line and is in particular a model for adenocarcinoma, especially COX-2 positive adenocarcinoma. The HT29 cell line was grown in McCoy's 5A modified medium supplemented with 1.5 mM L-glutamine, 10% FBS (fetal bovine serum) and 1% penicillin/streptomycin. The cells were grown in a humidified atmosphere containing 5% CO₂.

Cell proliferation was measured using the VybrantMTT assay kit according to the manufacturer's guidelines. HT29 cells (10,000 cells) cells were plated in 100 μl of complete McCoy's 5A media in 96-well tissue culture dishes. After 24 h cells were treated with celecoxib (celecoxib liquid formulations, Celebrex® and celecoxib dissolved in molecular grade DMSO) at 20, 30, 50 and 100 μM (n=6 for each concentration). In the case of celecoxib dissolved in DMSO, the celecoxib was dissolved overnight in DMSO before treatment. Two control groups were employed, one which involved no treatment (referred to as media) and the other which involved treatment with DMSO alone. The working concentration of DMSO in all treatments involving DMSO was <0.1%. After 72 h, the MTT [3-(4,5-dimethylthiazol-2-yl)-2,5-diphenyl-2H-tetrazolium bromide] labelling reagent (10 μl) was added and incubated for 3 h, followed by solubilisation with DMSO. The absorbance (A) was determined in a BioRad 680 microplate reader (BioRad, USA) at 550 nm.

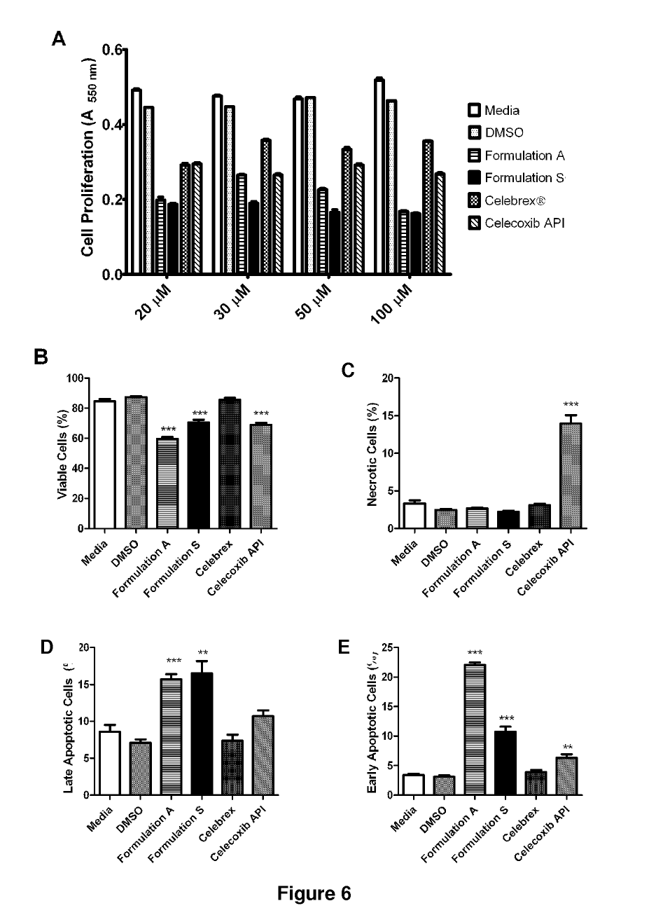

Liquid formulations containing celecoxib dissolved in various vehicles were prepared as described in Example 1. Two liquid formulations (Formulation A and S—Table 7) were selected to assess their affect on the proliferation of HT29 colorectal cancer cells in comparison to the marketed celecoxib product Celebrex®. In addition to the two control groups (medium and DMSO), cells were treated with celecoxib formulations A and S, Celebrex® and celecoxib API at 20, 30, 50 and 100 μM for a period of 72 h. The results for the two control groups were comparable. It was observed that the IC₅₀ dose (dose required to reduce cell proliferation by 50% compared to medium) for formulation A and formulation S after 72 hrs was ≦20 μM, whereas Celebrex® or celecoxib API did not achieve an IC₅₀ across the range from 20-100 μM (FIG. 6A).

TABLE 7

| | % | % Gelatine | Oils/Surfactants (Ratios) |
|---|---|---|---|
| Formulation | Celecoxib | Phase | (% of Formulation) |
| Formulation A | 7 | — | Miglyol ® 810N:Kolliphor ® |
| (liquid) | | | HS-15 (35:65) (93) |
| Formulation S | 10 | — | Kolliphor ® HS-15 (90) |
| (liquid) | | | |

### Example 11

### Effects of Celecoxib Formulations on the Proliferation of HT29 Cells

HT29 cells were seeded at 5×10⁵ cells/mL in 10% FBS-supplemented medium prior to treatment with celecoxib at 50μM (celecoxib liquid formulations, Celebrex® and celecoxib dissolved in molecular grade DMSO). Two control groups were employed, one which involved no treatment (referred to as media) and the other which involved treatment with DMSO alone. Cell viability and the onset of apoptosis was monitored using an Annexin-PE Apoptosis Detection Kit which contains recombinant Annexin V-fluorochrome phycoerythrin (PE) conjugate and the vital dye 7-amino-actinomycin (7-AAD) followed by flow cytometry on a FACSCalibur system using CellQest software (BD Biosciences, USA) . Data for at least 10,000 cells were collected for each analysis made and two-dimensional plots of 7-AAD versus PE were generated.

Cell viability and the onset of apoptosis were assessed via flow cytometry. In addition to the two control groups (media and DMSO), cells were treated with celecoxib formulations A and S, Celebrex® and celecoxib API at 50 μM for a period of 72 h (n=6). The results for the two control groups were comparable. Relative to the control (media), it was observed that the % of viable cells for formulations A and S were significantly reduced whereas in the case of Celebrex® there was no change in the % of viable cells (FIG. 6B). The % viable cells in the case of formulation A (59.6±2.59%) was lower than that for formulation S (70.51%±3.99%). The % viable cells for celecoxib API (69.02±2.71%) was higher than that for formulation A but was comparable to formulation S, however the % of cells that died by necrosis for celecoxib API (13.96±2.48%) was significantly higher than for any of the other formulations tested. Interestingly the combined % of cells that died via apoptosis (late and early apoptosis) for formulation A and formulation S were 37.77±2.40% and 27.26±4.27% respectively, which was significantly higher that achieved for either Celebrex® (11.30±2.41%) or celecoxib API (17.02±1.63%). It was noted that in the case of formulations A and S, that the cell culture media remained transparent following treatment with the drug but that the media was cloudy in the case of treatments with Celebrex® and celecoxib dissolved in DMSO. It was also noted that the extent of precipitation for cells treated with celecoxib dissolved in DMSO appeared to be less than that for cells treated with Celebrex®.

### Example 12

### Effects of Celecoxib Formulations on the Motility of HT29 Cells

Cellular motility was measured by an in vitro scratch-wound healing assay. HT29 cells were seeded in six-well plates and incubated until they were 90% confluent. The monolayer of cells was scratched vertically down the plate with a sterile pipette tip and debris was removed from the culture by washing twice with PBS. Images were captured immediately after wounding with a Nikon Eclipse Ti optical inverted microscope with 4× objective (Nikon, Japan). The cells were then incubated in complete medium with or without celecoxib at 50 μM (celecoxib liquid formulations, Celebrex® and celecoxib dissolved in molecular grade DMSO). Two control groups were employed, one which involved no treatment (referred to as media) and the other which involved treatment with DMSO alone. Wound closure was monitored microscopically after the wound persisted for 72 h. Scratch width before and after healing was measured (n=9) and compared to the controls. The percentage wound closure between the wound edges were analysed using Nikon NIS microscope imaging software. The experiments were performed with a minimum of three replicates.

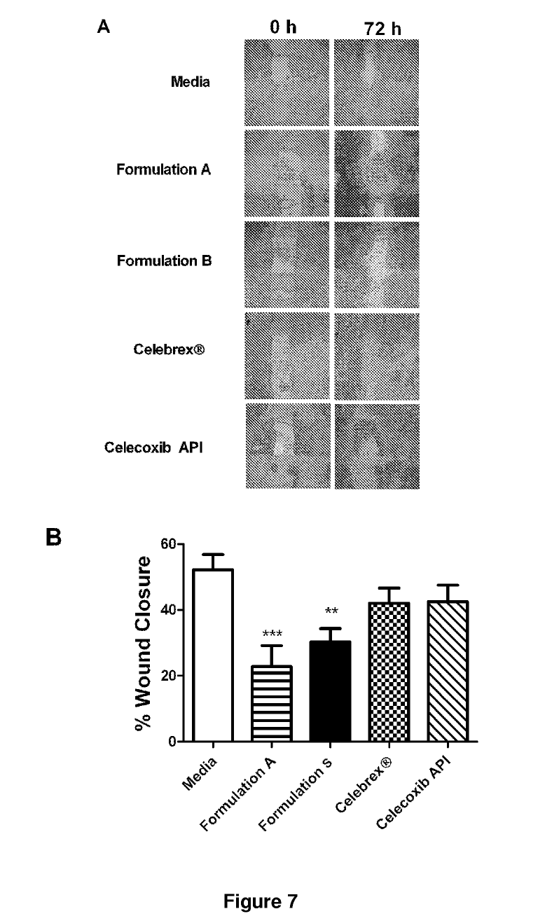

In order to examine the effects of celecoxib liquid formulations A and S on the motility of HT29 cells in comparison to Celebrex®, an in-vitro scratch-wound healing assay was performed. In addition to the two control groups (media and DMSO), cells were treated with celecoxib formulations A and S, Celebrex® and celecoxib API at 50 μM for a period of 72 h after the scratch wound was inflicted. The results for the two controls were comparable. FIG. 7A displays images of the wound on the day of application and after 72 h of incubation of the HT29 cells. As seen in the histogram in FIG. 7B, treatment with formulations A (p=0.0009) and S (p=0.001) significantly reduced the % wound closure relative to the control; indicating a loss in the motility of the HT29 cells after 72 h. In contrast the % wound closure for Celebrex® was not significant. Similar to the results from the cell viability and apoptosis experiments, this data also revealed a difference in the effect observed for formulations A and S.

### Example 13

### In-Vitro Drug Release Studies on Celecoxib Liquid Formulations

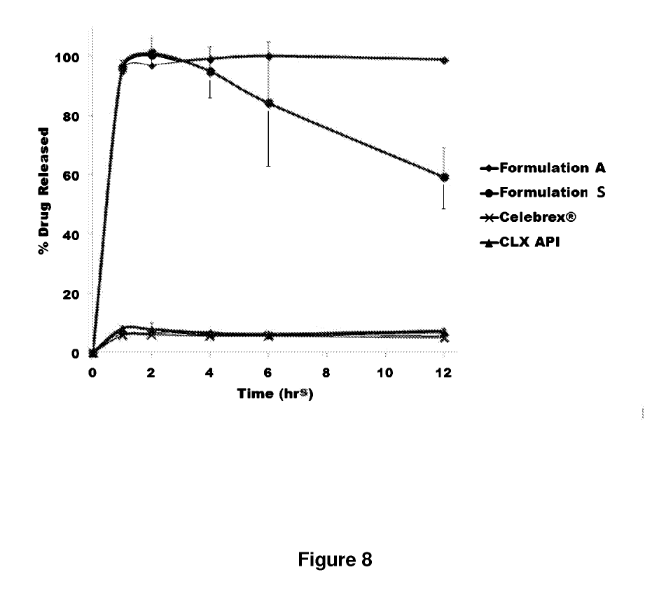

Given the differences in the effects observed for the celecoxib formulations compared to Celebrex® with respect to the in-vitro cell model parameters examined and also the different effects for the two liquid formulations (A and S), an in vitro dissolution study was performed to study the release of celecoxib and to assess whether a correlation existed between dissolution performance and the performance of the various formulations in the in-vitro cell model. FIG. 8 shows the in-vitro celecoxib release profiles from formulation A, formulation S, Celebrex® and celecoxib API (pre-dissolved in DMSO). Consistent with the in-vitro cell model data, formulations A and S significantly outperformed both Celebrex® and celecoxib API. Interestingly the % of release for formulation A remained steady over the 12 h period at 98.83±0.75%, whereas in the case of formulation S, celecoxib started to precipitate after 6 h and at 12 h the % of release had reduced to 59.23±9.90%.

### Example 14

### Coated Celecoxib Minibead Formulations

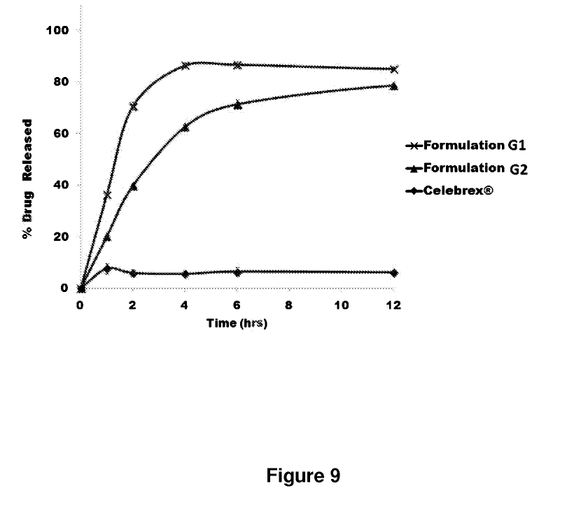

Following on from the in vitro cell model experiments, the liquid formulations were translated into an optimized minibead formulation (Formulation G of 2) as described in Examples 1-4. With the aim of developing a colonic targeted formulation for a future AOM/DSS CAC CRC murine study, a sustained release ethylcellulose polymer (Surelease®) was applied to the manually produced formulation G minibeads using a fluid bed wurster coating process with the intention of targeting the minibeads to the colon of the mouse. The target product profile (TPP) for the product to allow for colonic targeting was as follows; <10% release at 2 h, <50% release at 4 h and >80% release at 12 h. Formulation G was coated to yield two coated formulations (Table 5); formulation G1 (5% weight gain of Surelease®) and formulation G2 (8% weight gain of Surelease®). It can be seen from FIG. 9 (in-vitro dissolution release profiles for formulation G1 and formulation G2) that neither formulation met the desired TPP. As formulation G was manually produced, there was an insufficient quantity of minibeads to allow for the profile to be further optimized, therefore as a result, minibead production was scaled up using a vibrating nozzle, jet break-up encapsulator as described.

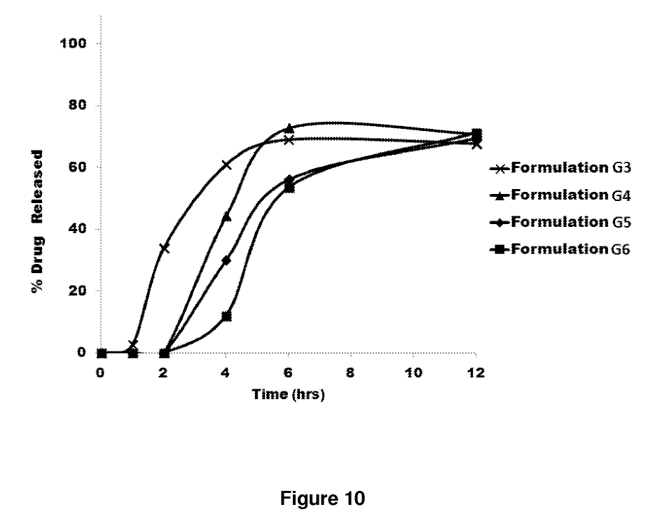

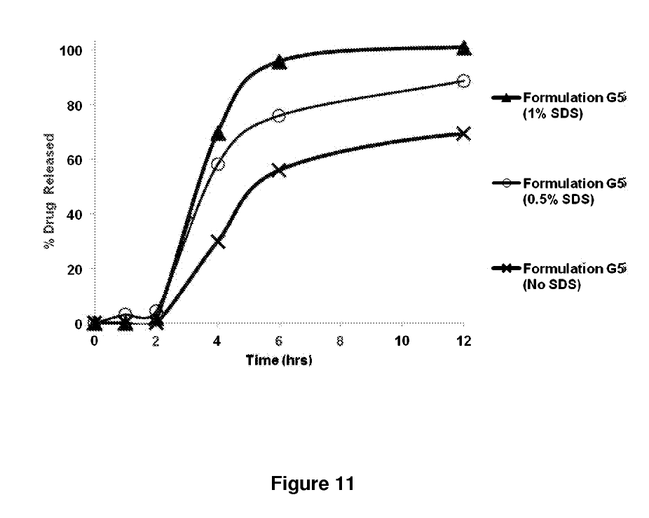

The automated production of minibeads allowed for a coating optimisation study resulting in formulations with between 17% and 32% weight gain of Surelease® (formulations G3, G4, G5 and G6 (Table 5)) which yielded a product (formulation G5-27% weight gain of Surelease®) which met the desired TPP at the 2 h and 4 h time points (FIG. 10). The % release at 12 h was 69.4% and therefore did not meet the TPP release specification. The dissolution media employed did not represent sink conditions for celecoxib (sink conditions for celecoxib requires 1% SDS and pH 12), therefore further dissolution experiments were performed on formulation G5, in which SDS was added to the dissolution media after 2 hrs. SDS is commonly used as a surfactant in dissolution media to mimic the use of natural surfactants such as lecithin and sodium taurocholate (known to present in intestinal fluids) which are prohibitively expensive. Whilst the FDA encourage the inclusions of surfactants in dissolution media for poorly soluble drug it also stipulates that dissolution experiments should be performed under physiological conditions were possible. For this reason, decreasing levels of SDS were employed (starting from 1% SDS) with the aim of keeping the dissolution media as biorelevant as possible whilst ensuring that the solubility of celecoxib did not become a limiting factor. A physiological pH of 6.8 was also maintained in all experiments. It can be seen from (FIG. 11) that the dissolution profile for Formulation G5 approached the desired TPP when an SDS concentration of 0.5% was employed. It is considered that the TPP would have been met at 0.25% and 0.1% SDS.

### Discussion

The first aim of Examples 10 to 13 was to compare the effects of liquid celecoxib formulations to the effect of Celebrex® on the proliferation and viability of a COX-2 positive colorectal cancer cell line, HT29. The results observed from the MTT assay (FIG. 6A) support a hypothesis that presenting celecoxib to the cells in a stable soluble form would allow for maximum distribution of celecoxib to the cells (and hence interaction with the cells) and consequently would allow for a greater inhibition of cellular proliferation: in the assay results, both celecoxib liquid formulations (A and S) inhibited HT29 proliferation (IC₅₀≦20 μM) to a much greater extent than the current marketed product Celebrex® (IC₅₀ not achieved across the range from 20 μM to 100 μM). It has been reported that the anti-carcinogenic effects of Celebrex® can only be observed at higher doses (800 mg/day) (Steinbach Get al. N Engl J Med 2000; 342(26):1946-1952) whereas the recommended dosage of Celebrex® for its anti-inflammatory indication is 200 mg/day. The MTT assay results presented here open the possibility for using a reduced dose of celecoxib to exert an anti-carcinogenic effect or an anti-inflammatory effect and a reduction or elimination of unwanted side effects.

In addition to the MTT proliferation results, a cell viability and apoptosis assay was performed on HT29 cells treated with celecoxib formulations at a concentration of 50 μM. As well as validating the MTT assay results (celecoxib formulations A and S were again demonstrated to be superior to Celebrex® with respect to their anti-proliferative effect on HT29 cells relative to a control), the data (FIG. 6B) also revealed interesting findings with respect to the mechanisms of inhibition. In the case of Celebrex®, in addition to the observation that there was no significant impact on cell viability, there was no significant necrotic (passive cell death) or apoptotic (controlled cell death) effect observed. There exists a theory that the cancer killing effect of celecoxib may be related to direct cytotoxicty (resulting from irreversible binding and damage to the plasma membrane by celecoxib precipitates) as opposed to molecular toxicity. Based on this theory, it could be argued that the concentration of Celebrex® used (50 μM) was not sufficient to result in direct cytotoxicty. In contrast, in the case of celecoxib API dissolved in DMSO, a significant necrotic effect was observed which is consistent with the direct cytotoxicity theory. In strong contrast to the results obtained for Celebrex® and celecoxib API, there was a very significant inhibitory effect observed for both formulation A and S without exerting a significant necrotic effect. The strong apoptotic effect for formulations A and S is highly significant, as it suggests that these formulations caused HT29 cell death via molecular mechanisms and thereby is a new development on the data presented by Sacchetti (Sacchetti, A. J Cell Biochem. 2013; 114(6):1434-44) in which he found that in-vitro cell death for CRC cell lines only occurred at insoluble concentrations of celecoxib. The data presented in the Examples suggests that a molecular toxic effect is possible for celecoxib if the drug is optimally presented to the cells (i.e., in a stable solubilised state). The finding that formulations A and S did not exhibit a significant necrotic effect is an important finding given that previous research by Tomisato et al. has shown that NSAIDs including celecoxib kill cells by both necrosis and apoptosis and that necrosis is linked to unwanted GI side effects. See Tomisato W et al. Biochem Pharmacol 2004; 67(3):575-585. It is inferred that the minicapsules of the invention will exert an anti-cancer activity with a surprisingly low level of GI side effects.

The high mortality associated with colorectal cancer is related to its ability to spread beyond the large intestine and invade distant sites. Therefore the metastatic potential of tumour cells (i.e., their ability to spread) is an extremely important factor for formation of solid tumours and necessary for their spread to distant organs. COX-2 expression is a hallmark in colon cancer cells with respect to increased metastatic potential (Greenhough A et al. Carcinogenesis 2009; 30(3):377-386), and NSAIDs have been shown to abrogate this invasiveness (Tsujii M et al. Proc Natl Acad Sci USA 1997; 94(7): 3336-3340). Example 11 examined the motility of HT29 cells as a measure of their metastatic potential via a scratch wound healing assay (FIG. 7). Cell motility was measured as a percentage of wound closure. As with the other assays performed, in contrast to Celebrex®, relative to the control, the celecoxib liquid formulations A and S had a significant effect, whereby the % wound closure was significantly reduced for formulations A and S (FIG. 7B), illustrating that these formulations had potential to reduce the potential of CRC cells to metastasise.

In the case of both the cell viability/apoptosis assay and the scratch wound healing assay, formulation A was observed to have had a superior effect compared to formulation S. An in-vitro dissolution test in purified water revealed a correlation between % of drug released and which remained in solution (FIG. 8) and the performance of the formulation with respect to the in-vitro cell culture model. This is an important finding as it identifies the use of dissolution testing in purified water as a simple and effective tool for screening and selecting stable formulations.

Subsequent to the in-vitro cell model, an optimised minibead formulation was developed and a sustained release polymer was applied. The TPP for the coated formulation included a requirement for 0% release at 2 h in order to ensure that the celecoxib would not be released from the minibead formulation prior to reaching the colon in a future in-vivo study in the mouse (Padmanabhan P et al. EJNMMI Research, 2013; 3(60): 1-8.). Due to the limited quantity of manually produced minibeads available for coating trials, initially the coated formulation resulted in dissolution profiles consistent with only partial colonic release. The next phase of the project involved the scale up of formulation G using automated encapsulation equipment which yielded batch sizes in the region of 50 g and therefore provided sufficient material for further coating optimisation studies. These coating studies allowed for the development of a fully colonic targeted celecoxib minibead which approached the desired TPP at 0.5% SDS (FIG. 11—Formulation G5).

Whilst it is generally accepted that the GI side effects of NSAIDs are mainly caused by COX-1 inhibition, which blocks the production of the protective mucus at the epithelial layer in the GI tract, COX-2 inhibitors such as celecoxib are still associated with GI toxicity (Silverstein F E et al. JAMA 2000; 284(10):1247-1255). This GI toxicity is partly attributable to local effects on the GI tract, including local irritation. Direct damage of the mucus layer and cytotoxicity to the GI epithelia has been observed for NSAIDs, including celecoxib. See Tomisato W et al. (see above) and Lichtenberger L M et al. J Pharm Pharmacol 2006; 58(11):1421-1428. Multiparticulate drug delivery systems have been shown to be less likely than single unit dosage forms to cause local irritation (Tang E S K et al. Am J Drug Deliv 2005; 3(1):17-28.) as they allow for a greater distribution of the drug. Similarly, micelle formulations allow for an even distribution of drug in the GI tract and can reduce the toxicity caused by the administration of a neat drug (Lui R et al. Micellization and Drug Solubility Enhancement. In: Lui R, 2^{nd} ed. Water-Insoluble Drug Formulation. New York: CRC Press, 2008: 255-306). The present invention therefore provides a way of presenting celecoxib in a micellar format within a multiparticulate, a combination that has the potential to minimize GI side effects whilst maintaining the effectiveness of the treatment. Furthermore the potential of employing a lower dose (a possibility based on the in-vitro cell culture results presented) furthermore highlights the potential for an improved celecoxib formulation with both reduced CV and GI side effects.

1. A composition that is a clear liquid comprising water, a hydrogel-forming polymer, celecoxib, a polyoxyethylated non-ionic surfactant and a precipitation inhibitor, wherein the celecoxib is in an amount of at least 2% and the polyoxyethylated non-ionic surfactant is in an amount of at least 40%, wherein the percentages are by weight, calculated on the dry weight of the total composition.

2. The composition of claim 1 that optionally comprises 2-(2-ethoxyethoxy)ethanol, any 2-(2-ethoxyethoxy)ethanol present being in an amount of at most 5% by weight of 2-(2-thoxyethoxy)ethanol, that is free of triglycerides, and wherein the polyoxyethylated non-ionic surfactant is in an amount from 50% to 60%, wherein the percentages are by weight based on the dry weight of the composition.

3. The composition of claim 1 that further comprises a plasticiser for the polymer, wherein the precipitation inhibitor is an anionic surfactant or a cellulose polymer and the composition consists essentially of the constituents in the amounts stated in the following table for any of the formulations designated A to F:

| | A | B | C | D | E | F |
|---|---|---|---|---|---|---|
| Constituent | Weight % | Weight % | Weight % | Weight % | Weight % | Weight % |
| Celecoxib | 2-10 | 2-10 | 4-10 | 4-9 | 5-9 | 5-7 |
| Non-ionic | 40-70 | 45-70 | 50-65 | 50-60 | 52.5-57.5 | 52.5-57.5 |
| surfactant | | | | | | |
| Polymer | 25-40 | 25-40 | 25-37.5 | 25-35 | 28-35 | 30-35 |
| Anionic | 0.5-10 | 1-5 | 1-5 | 3-5 | 3-5 | 3-5 |
| surfactant or | | | | | | |
| Cellulose | 0.5-10 | 0.5-3 | 0.5-3 | 0.5-3 | 0.5-2 | 0.5-2 |
| polymer | | | | | | |
| product | | | | | | |
| Plasticiser | 0-8 | 2-8 | 2-8 | 2-6 | 2-6 | 2-6 |

wherein: the above percentages are by dry weight; in Formula B the amount of the hydrogel-forming polymer is from 28-40 when the amount of the non-ionic surfactant is from 45-67; and the total percentage contents of the constituents excluding water add up to 100.

4. The composition of claim 1 wherein the composition further comprsises a plasticiser and the identities of the constituents of the composition are as set out in one of Columns (a) to (f) of the following table or as set out in a variant of Column (f) in which the precipitation inhibitor is as defined in Column (e):

| | (a) | (b) | (c) | (d) | (e) | (f) |
|---|---|---|---|---|---|---|
| Constituent | Identity | Identity | Identity | Identity | Identity | Identity |
| Non-ionic | Comprises a | Comprises | Polyoxyl | Polyoxyl | Polyoxyl- | Polyoxyl- |
| surfactant | polyoxyethyl | a polyoxy- | fatty acid | hydroxy fatty | 15- | 15- |
| | ester or | ethylated | (macrogol | acid | hydroxy- | hydroxy- |
| | ether | aliphatic | ester of fatty | | stearate | stearate |
| | | acid | acid) | | | |
| Polymer | Thermo- | Thermo- | At least one | Gelatin; and | Gelatin | Gelatin |
| | reversible | reversible | of gelatin; | optionally a | | |
| | hydrogel- | hydrogel- | agar; | minor | | |
| | forming | forming | agarose; | proportion of | | |
| | polymer(s) | polymer(s), | pectin; | agar; | | |
| | | gelatin at | carrageenan; | agarose; | | |
| | | least | chitosan. | pectin; | | |
| | | predominating | | carrageenan; | | |
| | | | | and/or | | |
| | | | | chitosan. | | |
| Precipitation | An anionic | An anionic | An aliphatic | An alkyl | A dodecyl | Sodium |
| Inhibitor | surfactant; | surfactant; | sulfate or | sulfate; or a | sulfate; or | dodecyl |
| | or a product | or a | sulfonate; or | product that | HPC | sulfate; or |
| | that | product | a product | comprises | and/or | HPC and |
| | comprises a | that | that | HPC and/or | HPMC, | HPMC |
| | cellulose | comprises | comprises | HPMC | optionally | optionally |
| | polymer | HPC | HPC and/or | | together | together |
| | | and/or | HPMC | | with (i) | with (i) |
| | | HPMC | | | TiO₂ | TiO₂ |
| | | | | | and/or (ii) | and/or (ii) |
| | | | | | talc, e.g. | talc, e.g. |
| | | | | | with both | with both |
| | | | | | (i) and (ii) | (i) and (ii) |
| Plasticiser | At least one | At least | At least one | At least one | Sorbitol | Sorbitol |
| | polyol. | one of | of glycerol, | of glycerol, | | |
| | | glycerol, | sorbitol, | sorbitol, | | |
| | | sorbitol, | sorbitol/ | sorbitol/ | | |
| | | sorbitol/ | sorbitan | sorbitan | | |
| | | sorbitan | mixture | mixture | | |
| | | mixture | | | | |

5. A process of making minibeads, comprising ejecting the composition of claim 1 through a nozzle to form droplets, the hydrogel-forming polymer then being caused or allowed to solidify whereby the droplets form minibeads, optionally wherein the minibeads are then dried.

6. The process of claim 5 that further comprises coating the minibeads.

7. The process of claim 6 wherein the coating is adapted for the minibeads to release the celecoxib at least in the colon.

8. The process of claim 5 wherein the minibeads have a size of from 0.5 mm to 5 mm.

9. The process of claim 5 that further comprises making the composition ejected through the nozzle, the making of the composition comprising mixing:
i) a celecoxib solution comprising the celecoxib and the polyoxyethylated non-ionic surfactant; and
ii) an aqueous solution comprising water, the hydrogel-forming polymer and the precipitation inhibitor;
wherein the two solutions are mixed to form a clear liquid.

10. An optionally coated minibead having the characteristics of a minibead obtained by the process of claim 5.

11. A composition comprising celecoxib in an amount of at least 2%, a polyoxyethylated non-ionic surfactant in an amount of at least 40%, a precipitation inhibitor, and a hydrogel-forming polymer matrix in which the celecoxib and the surfactant are included, the composition optionally being coated and the percentages being by weight, calculated on the dry weight of the composition excluding any coating(s).

12. A composition of claim 11 wherein the composition has a feature selected from:
(i) when combined with water, the composition is capable of releasing self-assembly structures comprising non-ionic surfactant and celecoxib; or
(ii) the polymer forms a matrix in which the polyoxyethylated non-ionic surfactant is distributed as inclusions that comprise celecoxib and have a size of less than 25 μm; or
(iii) (i) and (ii) together.

13. The composition of claim 11 that comprises at most 5% by weight, calculated on the dry weight of the composition excluding any coating(s), of 2-(2-ethoxyethoxy)ethanol.

14. The composition of claim 11 that comprises at most 5% by weight, calculated on the dry weight of the composition excluding any coating(s), of triglycerides.

15. The composition of claim 11 wherein the polyoxyethylated non-ionic surfactant is in an amount of at least 40% by weight based on the dry weight of the composition excluding any coating(s).

16. The composition of claim 15 wherein the polyoxyethylated non-ionic surfactant is in an amount from 45% to 70% by weight based on the dry weight of the composition excluding any coating(s).

17. The composition of claim 11 that comprises at most 5% by weight of 2-(2-ethoxyethoxy)ethanol, that is free of triglycerides, and wherein the polyoxyethylated non-ionic surfactant is in an amount from 50% to 60%, wherein the percentages are by weight based on the dry weight of the composition excluding any coating(s).

18. The composition of claim 11 wherein the weight ratio of the hydrogel-forming polymer to the polyoxyethylated non-ionic surfactant is from 1:1.5 to 1:2.1.

19. The composition of claim 11 wherein the precipitation inhibitor is in an amount of from 0.5% to 10%, wherein the percentages are by weight based on the dry weight of the composition excluding any coating(s).

20. The composition of claim 11 wherein the hydrogel-forming polymer is selected from a thermotropic hydrogel-forming polymer and a combination thereof.

21. The composition of claim 20 wherein the hydrogel-forming polymer is selected from the group consisting of gelatin, agar, agarose, pectin, carrageenan, and chitosan, and a combination thereof.

22. The composition of claim 11 wherein the hydrogel-forming polymer is gelatin.

23. The composition of claim 11 wherein the composition comprises the hydrogel-forming polymer in an amount of from 25% to 40%, wherein the percentages are by weight based on the dry weight of the composition excluding any coating(s).

24. The composition of claim 11 wherein the polyoxyethylated non-ionic surfactant comprises a combination of a polyoxyethylated aliphatic acid and free polyethylene glycol.

25. The composition of claim 24 wherein the polyoxyethylated non-ionic surfactant comprises polyoxyl-15-hydroxystearate.

26. The composition of claim 11 wherein the precipitation inhibitor comprises an anionic surfactant or a cellulose polymer, or a combination thereof.

27. The composition of claim 26 wherein the precipitation inhibitor comprises hydroxypropyl cellulose and/or hydroxypropylmethyl cellulose.

28. The composition of claim 27 wherein the precipitation inhibitor comprises a mixture of hydroxypropyl cellulose, hydroxypropylmethyl cellulose, talc and TiO₂.

29. The composition of claim 28 wherein the precipitation inhibitor comprises an anionic surfactant.

30. The composition of claim 29 wherein the anionic surfactant is sodium dodecyl sulfate (SDS).

31. The composition of claim 11 wherein the weight ratio of the anionic surfactant to celecoxib is from 1:1 to 1:2.3.

32. The composition of claim 26 wherein the precipitation inhibitor comprises a cellulose polymer and wherein the weight ratio of the precipitation inhibitor to celecoxib is from 1:3 to 1:8.

33. The composition of claim 11 wherein the weight ratio of the polyoxyethylated non-ionic surfactant to celecoxib is from 7.5:1 to 11.5:1.

34. The composition of claim 11 wherein the celecoxib is in an amount of 4 to 9% by weight based on the dry weight of the composition excluding any coating(s).

35. The composition of claim 11 that further comprises a plasticiser for the polymer, wherein the precipitation inhibitor is an anionic surfactant or a cellulose polymer and the composition consists essentially of the constituents in the amounts stated in the following table for any of the formulations designated A to F:

| | A | B | C | D | E | F |
|---|---|---|---|---|---|---|
| Constituent | Weight % | Weight % | Weight % | Weight % | Weight % | Weight % |
| Celecoxib | 2-10 | 2-10 | 4-10 | 4-9 | 5-9 | 5-7 |
| Non-ionic | 40-70 | 45-70 | 50-65 | 50-60 | 52.5-57.5 | 52.5-57.5 |
| surfactant | | | | | | |
| Polymer | 25-40 | 25-40 | 25-37.5 | 25-35 | 28-35 | 30-35 |
| Anionic | 0.5-10 | 1-5 | 1-5 | 3-5 | 3-5 | 3-5 |
| surfactant or | | | | | | |
| Cellulose | 0.5-10 | 0.5-3 | 0.5-3 | 0.5-3 | 0.5-2 | 0.5-2 |
| polymer | | | | | | |
| product | | | | | | |
| Plasticiser | 0-8 | 2-8 | 2-8 | 2-6 | 2-6 | 2-6 |

wherein: the above percentages are by dry weight excluding any coating(s); in Formula B the amount of the hydrogel-forming polymer is from 28-40 when the amount of the non-ionic surfactant is from 45-67; and the total percentage contents of the constituents excluding water add up to 100.

36. The composition of claim 11 that further comprises a plasticiser for the polymer and wherein the identities of the constituents of the , are as set out in one of Columns (a) to (f) of the following table or as set out in a variant of Column (f) in which the precipitation inhibitor is as defined in Column (e):

| | (a) | (b) | (c) | (d) | (e) | (f) |
|---|---|---|---|---|---|---|
| Constituent | Identity | Identity | Identity | Identity | Identity | Identity |
| Non-ionic | Comprises a | Comprises | Polyoxyl fatty | Polyoxyl | Polyoxyl- | Polyoxyl- |
| surfactant | polyoxy- | a polyoxy- | acid | hydroxy fatty | 15- | 15- |
| | ethyl ester | ethylated | (macrogol | acid | hydroxy- | hydroxy- |
| | or ether | aliphatic | ester of fatty | | stearate | stearate |
| | | acid | acid) | | | |
| Polymer | Thermo- | Thermo- | At least one | Gelatin; and | Gelatin | Gelatin |
| | reversible | reversible | of gelatin; | optionally a | | |
| | hydrogel- | hydrogel- | agar; | minor | | |
| | forming | forming | agarose; | proportion of | | |
| | polymer(s) | polymer(s), | pectin; | agar; | | |
| | | gelatin at | carrageenan; | agarose; | | |
| | | least | chitosan. | pectin; | | |
| | | predominating | | carrageenan; | | |
| | | | | and/or | | |
| | | | | chitosan. | | |
| Precipitation | An anionic | An anionic | An aliphatic | An alkyl | A dodecyl | Sodium |
| Inhibitor | surfactant; | surfactant; | sulfate or | sulfate; or a | sulfate; or | dodecyl |
| | or a | or a | sulfonate; or | product that | HPC | sulfate; or |
| | product | product | a product | comprises | and/or | HPC and |
| | that | that | that | HPC and/or | HPMC, | HPMC |
| | comprises | comprises | comprises | HPMC | optionally | optionally |
| | a cellulose | HPC | HPC and/or | | together | together |
| | polymer | and/or | HPMC | | with (i) | with (i) |
| | | HPMC | | | TiO₂ | TiO₂ |
| | | | | | and/or (ii) | and/or (ii) |
| | | | | | talc, e.g. | talc, e.g. |
| | | | | | with both | with both |
| | | | | | (i) and (ii) | (i) and (ii) |
| Plasticiser | At least | At least | At least one | At least one | Sorbitol | Sorbitol |
| | one | one of | of glycerol, | of glycerol, | | |
| | polyol. | glycerol, | sorbitol, | sorbitol, | | |
| | | sorbitol, | sorbitol/ | sorbitol/ | | |
| | | sorbitol/ | sorbitan | sorbitan | | |
| | | sorbitan | mixture, e.g. | mixture, e.g. | | |
| | | mixture, | sorbitol. | sorbitol. | | |
| | | e.g. | | | | |
| | | sorbitol. | | | | |

37. The composition of claim 11 wherein the precipitation inhibitor at least predominantly shares the same space as the hydrogel-forming polymer.

38. The composition of claim 11 that is suitable for oral administration and that further comprises at least one coating.

39. The composition of claim 38 wherein the at least one coating is adapted to release the celecoxib in at least the colon.

40. The composition of claim 39 that is adapted to release the celecoxib in the ileum and colon or that is adapted to release substantially all the celecoxib in the colon.

41. The composition of claim 11 that is in the form of a minibead having a size of from 0.5 mm to 5 mm.

42. A multiple minibead formulation, comprising a unit dosage form comprising a multiplicity of minibeads of claim 41.

43. A celecoxib formulation adapted for oral administration, the formulation being a multiple minibead formulation wherein the minibeads comprise a hydrogel-forming polymer matrix in which are distributed celecoxib in an amount of at least 2%, a polyoxyethylated non-ionic surfactant and a precipitation inhibitor in an amount of at least 40%, the minibeads optionally being coated and the percentages being by weight, calculated on the dry weight of the hydrogel-forming polymer matrix and substances in the matrix.

44. A process which comprises mixing a polyoxyethylated non-ionic surfactant and celecoxib to form a solution, wherein the weight ratio of the polyoxyethylated non-ionic surfactant to celecoxib is from 7.5:1 to 11.5:1, the process optionally further comprising mixing the solution with an aqueous solution comprising water, a hydrogel-forming polymer and a precipitation inhibitor to form a clear liquid.

45. A product that comprises a mixture of celecoxib and a polyoxyethylated non-ionic surfactant, wherein the weight ratio of the polyoxyethylated non-ionic surfactant to celecoxib is from 7.5:1 to 11.5:1.

46. A process of making a celecoxib composition, comprising mixing:
i) a celecoxib solution comprising celecoxib and a polyoxyethylated non-ionic surfactant; and
ii) an aqueous solution comprising water, a hydrogel-forming polymer and an precipitation inhibitor;
wherein the two solutions are mixed to form a clear liquid, the celecoxib and the non-ionic surfactant being in amounts such that the clear liquid comprises celecoxib in an amount of at least 2% and the non-ionic surfactant in an amount of at least 40% wherein the percentages are by weight, calculated on the dry weight of the total clear liquid.

47. The process of claim 46 that further comprises ejecting the clear liquid through a nozzle to form droplets, the hydrogel-forming polymer then being caused or allowed to solidify whereby the droplets form minibeads, optionally wherein the minibeads are then dried, the minibeads optionally being coated to form at least one coating.

48. A minibead having the characteristics of a minibead made by the process of claim 47.

49. A method for killing colorectal cancer cells in a patient, comprising administering to the patient a product, in which method apoptosis is favoured over necrosis, wherein the product is selected from:
a. an optionally coated minibead having the characteristics of a minibead obtained by a process comprising ejecting a composition through a nozzle to form droplets, wherein the compsotion is a clear liquid comprising water, a hydrogel-forming polymer, celecoxib, a polyoxyethylated non-ionic surfactant and a precipitation inhibitor, wherein the celecoxib is in an amount of at least 2% and the polyoxyethylated non-ionic surfactant is in an amount of at least 40%, wherein the percentages are by weight, calculated on the dry weight of the total composition, hydrogel-forming polymer then being caused or allowed to solidify whereby the droplets form minibeads, optionally wherein the minibeads are then dried;
b. a composition comprising celecoxib in an amount of at least 2%, a polyoxyethylated non-ionic surfactant in an amount of at least 40%, a precipitation inhibitor, and a hydrogel-forming polymer matrix in which the celecoxib and the surfactant are included, the composition optionally being coated and the percentages being by weight, calculated on the dry weight of the composition excluding any coating(s);
c. a unit dosage form comprising a multiplicity of minibeads having a size of from 0.5 mm to 5 mm wherein the minibeads are the composition of (ii) in the form of minibeads ;
d. a celecoxib formulation adapted for oral administration, the formulation being a multiple minibead formulation wherein the minibeads comprise a hydrogel-forming polymer matrix in which are distributed celecoxib in an amount of at least 2%, a polyoxyethylated non-ionic surfactant and a precipitation inhibitor in an amount of at least 40%, the minibeads optionally being coated and the percentages being by weight, calculated on the dry weight of the hydrogel-forming polymer matrix and substances in the matrix; or
e. the minibead of claim 48.

50. A method for treating colorectal cancer in a subject, comprising administering to the subject a product selected from:
a. an optionally coated minibead having the characteristics of a minibead obtained by a process comprising ejecting a composition through a nozzle to form droplets, wherein the compsotion is a clear liquid comprising water, a hydrogel-forming polymer, celecoxib, a polyoxyethylated non-ionic surfactant and a precipitation inhibitor, wherein the celecoxib is in an amount of at least 2% and the polyoxyethylated non-ionic surfactant is in an amount of at least 40%, wherein the percentages are by weight, calculated on the dry weight of the total composition, the hydrogel-forming polymer then being caused or allowed to solidify whereby the droplets form minibeads, optionally wherein the minibeads are then dried;
b. a composition comprising celecoxib in an amount of at least 2%, a polyoxyethylated non-ionic surfactant in an amount of at least 40%, a precipitation inhibitor, and a hydrogel-forming polymer matrix in which the celecoxib and the surfactant are included, the composition optionally being coated and the percentages being by weight, calculated on the dry weight of the composition excluding any coating(s);
c. a unit dosage form comprising a multiplicity of minibeads having a size of from 5 mm to 5 mm wherein the minibeads are the composition of (ii) in the form of minibeads;
d. a celecoxib formulation adapted for oral administration, the formulation being a multiple minibead formulation wherein the minibeads comprise a hydrogel-forming polymer matrix in which are distributed celecoxib in an amount of at least 2%, a polyoxyethylated non-ionic surfactant and a precipitation inhibitor in an amount of at least 40%, the minibeads optionally being coated and the percentages being by weight, calculated on the dry weight of the hydrogel-forming polymer matrix and substances in the matrix; or
e. the minibead of claim 48.

51. The method of claim 50 wherein the colorectal cancer is a COX-2 positive adenocarcinoma.

52. The method of claim 50 wherein the celecoxib is administered at a daily dosage of less than 800 mg/day.

53. The method of claim 50 wherein the celecoxib is administered at a daily dosage of no more than 500 mg/day.

54. The method of claim 50 wherein the celecoxib is administered at a daily dosage of less than 200 mg/day.

55. The method of claim 50 wherein the celecoxib is administered at a daily dosage of no more than 100 mg/day.

56. A method for treating colorectal inflammation or other gastrointestinal inflammatory condition in a subject, comprising administering to the subject a product selected from:
a. an optionally coated minibead having the characteristics of a minibead obtained by a process comprising ejecting a composition through a nozzle to form droplets, wherein the compsotion is a clear liquid comprising water, a hydrogel-forming polymer, celecoxib, a polyoxyethylated non-ionic surfactant and a precipitation inhibitor, wherein the celecoxib is in an amount of at least 2% and the polyoxyethylated non-ionic surfactant is in an amount of at least 40%, wherein the percentages are by weight, calculated on the dry weight of the total composition, hydrogel-forming polymer then being caused or allowed to solidify whereby the droplets form minibeads, optionally wherein the minibeads are then dried;
b. a composition comprising celecoxib in an amount of at least 2%, a polyoxyethylated non-ionic surfactant in an amount of at least 40%, a precipitation inhibitor, and a hydrogel-forming polymer matrix in which the celecoxib and the surfactant are included, the composition optionally being coated and the percentages being by weight, calculated on the dry weight of the composition excluding any coating(s);
c. a unit dosage form comprising a multiplicity of minibeads having a size of from 5 mm to 5 mm wherein the minibeads are the composition of (ii) in the form of minibeads;
d. a celecoxib formulation adapted for oral administration, the formulation being a multiple minibead formulation wherein the minibeads comprise a hydrogel-forming polymer matrix in which are distributed celecoxib in an amount of at least 2%, a polyoxyethylated non-ionic surfactant and a precipitation inhibitor in an amount of at least 40%, the minibeads optionally being coated and the percentages being by weight, calculated on the dry weight of the hydrogel-forming polymer matrix and substances in the matrix; or
e. the minibead of claim 48.

57. The method of claim 56, wherein the method inhibits, reduces or delays the initiation and/or progression of the inflammatory condition, or causes regression of or reduces the inflammatory condition.

58. The method of claim 56, wherein the celecoxib is administered at a daily dosage of less than 200 mg/day.

59. The method of claim 56, wherein the celecoxib is administered at a daily dosage of no more than 100 mg/day.

60. The method of claim 56, wherein the subject suffers from intolerance to another NSAID or has a history of hypersensitivity to another NSAID.

61. The method of claim 56, which further comprises administering another drug selected from anti-inflammatory drugs, immunosuppressants and drugs for the therapy or prophylaxis of cancer.

62. A method of inhibiting, reducing or delaying metastasis of a colorectal cancer in a subject, comprising administering to the subject:
a. an optionally coated minibead having the characteristics of a minibead obtained by a process comprising ejecting a composition through a nozzle to form droplets, wherein the compsotion is a clear liquid comprising water, a hydrogel-forming polymer, celecoxib, a polyoxyethylated non-ionic surfactant and a precipitation inhibitor, wherein the celecoxib is in an amount of at least 2% and the polyoxyethylated non-ionic surfactant is in an amount of at least 40%, wherein the percentages are by weight, calculated on the dry weight of the total composition, the hydrogel-forming polymer then being caused or allowed to solidify whereby the droplets form minibeads, optionally wherein the minibeads are then dried;
b. a composition comprising celecoxib in an amount of at least 2%, a polyoxyethylated non-ionic surfactant in an amount of at least 40%, a precipitation inhibitor, and a hydrogel-forming polymer matrix in which the celecoxib and the surfactant are included, the composition optionally being coated and the percentages being by weight, calculated on the dry weight of the composition excluding any coating(s);
c. a unit dosage form comprising a multiplicity of minibeads having a size of from 0.5 mm to 5 mm wherein the minibeads are the composition of (ii) in the form of minibeads;
d. a celecoxib formulation adapted for oral administration, the formulation being a multiple minibead formulation wherein the minibeads comprise a hydrogel-forming polymer matrix in which are distributed celecoxib in an amount of at least 2%, a polyoxyethylated non-ionic surfactant and a precipitation inhibitor in an amount of at least 40%, the minibeads optionally being coated and the percentages being by weight, calculated on the dry weight of the hydrogel-forming polymer matrix and substances in the matrix; or
e. the minibead of claim 48.

63. The method of claim 62, wherein the method reduces the risk of the metastasis.
